SEQUENCE LISTING (128 sequences)

SEQ ID NO: 1 - protein, 5 aa, synthetic construct
SYWMH

SEQ ID NO: 2 - protein, 17 aa, synthetic construct
RINSDGSSTTYADSVKG

SEQ ID NO: 3 - protein, 14 aa, synthetic construct
DRGWGGYYYYGMDV

SEQ ID NO: 4 - protein, 5 aa, synthetic construct
SYWMH

SEQ ID NO: 5 - protein, 17 aa, synthetic construct
RINSDGSSTSYADSVKG

SEQ ID NO: 6 - protein, 14 aa, synthetic construct
DRGWGGYYYYGMDV

SEQ ID NO: 7 - protein, 7 aa, synthetic construct
SGGYYWS

SEQ ID NO: 8 - protein, 16 aa, synthetic construct
YIFYSGSTYYNPSLKS

SEQ ID NO: 9 - protein, 14 aa, synthetic construct
AAAGRGYYYYGLDV

SEQ ID NO: 10 - protein, 5 aa, synthetic construct
NYWMH

SEQ ID NO: 11 - protein, 17 aa, synthetic construct
RINSDGSSTSYADSVKG

SEQ ID NO: 12 - protein, 22 aa, synthetic construct
DRDYYGSGSYYNVGYYYYGMDV

SEQ ID NO: 13 - protein, 5 aa, synthetic construct
SYWMH

SEQ ID NO: 14 - protein, 17 aa, synthetic construct
RINSDGSSTSYADSVKG

SEQ ID NO: 15 - protein, 22 aa, synthetic construct
DRDYSGSGSYYNVGYYYYGMDV

SEQ ID NO: 16 - protein, 7 aa, synthetic construct
SFINYWG

SEQ ID NO: 17 - protein, 16 aa, synthetic construct
NIYYSGSTYYNPSLKS

SEQ ID NO: 18 - protein, 16 aa, synthetic construct
HGRRGPYGGYYYGMDV

SEQ ID NO: 19 - protein, 5 aa, synthetic construct
SYWMH

SEQ ID NO: 20 - protein, 17 aa, synthetic construct
RINSDGSSTSYADSVKG

SEQ ID NO: 21 - protein, 22 aa, synthetic construct
DRDYYGSGSYYNVGYYYYGMDV

SEQ ID NO: 22 - protein, 7 aa, synthetic construct
SSSYYWG

SEQ ID NO: 23 - protein, 16 aa, synthetic construct
SIYYSGSTYYNPSLKS

SEQ ID NO: 24 - protein, 14 aa, synthetic construct
HNWGRGYYYYGMDV

SEQ ID NO: 25 - protein, 7 aa, synthetic construct
SSSYYWG

SEQ ID NO: 26 - protein, 16 aa, synthetic construct
SIYYSGSTYYNPSLKS

SEQ ID NO: 27 - protein, 16 aa, synthetic construct
LGPAAIGYSYYYGMDV

SEQ ID NO: 28 - protein, 7 aa, synthetic construct
GFTFSSY

SEQ ID NO: 29 - protein, 6 aa, synthetic construct
NSDGSS

SEQ ID NO: 30 - protein, 14 aa, synthetic construct
DRGWGGYYYYGMDV

SEQ ID NO: 31 - protein, 9 aa, synthetic construct
GGSISSGGY

SEQ ID NO: 32 - protein, 5 aa, synthetic construct
FYSGS

SEQ ID NO: 33 - protein, 14 aa, synthetic construct
AAAGRGYYYYGLDV

SEQ ID NO: 34 - protein, 7 aa, synthetic construct
GFTFSNY

SEQ ID NO: 35 - protein, 6 aa, synthetic construct
NSDGSS

SEQ ID NO: 36 - protein, 22 aa, synthetic construct
DRDYYGSGSYYNVGYYYYGMDV

SEQ ID NO: 37 - protein, 7 aa, synthetic construct
GFTFSSY

SEQ ID NO: 38 - protein, 6 aa, synthetic construct
NSDGSS

SEQ ID NO: 39 - protein, 22 aa, synthetic construct
DRDYSGSGSYYNVGYYYYGMDV

SEQ ID NO: 40 - protein, 9 aa, synthetic construct
GGSISSFIN

SEQ ID NO: 41 - protein, 5 aa, synthetic construct
YYSGS

SEQ ID NO: 42 - protein, 16 aa, synthetic construct
HGRRGPYGGYYYGMDV

SEQ ID NO: 43 - protein, 7 aa, synthetic construct
GFTFSSY

SEQ ID NO: 44 - protein, 6 aa, synthetic construct
NSDGSS

SEQ ID NO: 45 - protein, 22 aa, synthetic construct
DRDYYGSGSYYNVGYYYYGMDV

SEQ ID NO: 46 - protein, 9 aa, synthetic construct
GGSISSSSY

SEQ ID NO: 47 - protein, 5 aa, synthetic construct
YYSGS

SEQ ID NO: 48 - protein, 14 aa, synthetic construct
HNWGRGYYYYGMDV

SEQ ID NO: 49 - protein, 9 aa, synthetic construct
GGSISSSSY

SEQ ID NO: 50 - protein, 5 aa, synthetic construct
YYSGS

SEQ ID NO: 51 - protein, 16 aa, synthetic construct
LGPAAIGYSYYYGMDV

SEQ ID NO: 52 - protein, 11 aa, synthetic construct
RASQGISSWLA

SEQ ID NO: 53 - protein, 7 aa, synthetic construct
AASSLQS

SEQ ID NO: 54 - protein, 9 aa, synthetic construct
QQANSFPLS

SEQ ID NO: 55 - protein, 11 aa, synthetic construct
RASQGISSWLA

SEQ ID NO: 56 - protein, 7 aa, synthetic construct
AASSLQS

SEQ ID NO: 57 - protein, 9 aa, synthetic construct
QQANSFPLT

SEQ ID NO: 58 - protein, 11 aa, synthetic construct
RASQGISSWLA

SEQ ID NO: 59 - protein, 7 aa, synthetic construct
AASSLQS

SEQ ID NO: 60 - protein, 9 aa, synthetic construct
QQANSFPYT

SEQ ID NO: 61 - protein, 123 aa, synthetic construct
QLQLQESGGGLVQPGGSLRLSCAASGFTFSSYWMHWVRQAPGKGLMWVSRINSDGSSTTYADSVKGRFTISRDNAKNTLYLQMNSLRAEDTAVYYCARDRGWGGYYYYGMDVWGQGTTVTVSS

SEQ ID NO: 62 - protein, 123 aa, synthetic construct
QVQLVESGGGLVQPGGSLRLSCAASGFTFSSYWMHWVRQAPGKGLVWVSRINSDGSSTSYADSVKGRFTISRDNAKNTLYLQMNSLRAEDTAVYYCARDRGWGGYYYYGMDVWGQGTTVTVSS

SEQ ID NO: 63 - protein, 124 aa, synthetic construct
QVQLQESGPGLVQPSQTLSLTCTVSGGSISSGGYYWSWIRQHPGKGLDWIGYIFYSGSTYYNPSLKSRLTISVDTSKNQFSLKLSSVTAADTAVYYCARAAAGRGYYYYGLDVWGQGTTVTVSS

SEQ ID NO: 64 - protein, 124 aa, synthetic construct
QVQLQQSGPGLVQPSQTLSLTCTVSGGSISSGGYYWSWIRQHPGKGLDWIGYIFYSGSTYYNPSLKSRLTISVDTSKNQFSLKLSSVTAADTAVYYCARAAAGRGYYYYGLDVWGQGTTVTVSS

SEQ ID NO: 65 - protein, 131 aa, synthetic construct
QVQLQESGGGLVQPGGSLRLSCAASGFTFSNYWMHWVRQAPGKGLVWVSRINSDGSSTSYADSVKGRFTISRDNAKNTLNLQMNSLRTEDTAVYYCARDRDYYGSGSYYNVGYYYYGMDVWGQGTTVTVSS

SEQ ID NO: 66 - protein, 131 aa, synthetic construct
QVQLQESGGGLVQPGGSLRLSCAASGFTFSSYWMHWVRQAPGKGLVWVSRINSDGSSTSYADSVKGRFTMSRDNAKNTQYLQMNSLRAEDTAVYYCARDRDYSGSGSYYNVGYYYYGMDVWGQGTTVTVSS

SEQ ID NO: 67 - protein, 126 aa, synthetic construct
QVQLQQSGPGLVKPSETLSLTCTVSGGSISSFINYWGWIRQPPGKGLEWIGNIYYSGSTYYNPSLKSRVTISVDTSKNQFSLKLSSVTAADTAVYYCARHGRRGPYGGYYYGMDVWGQGTTVTVSS

SEQ ID NO: 68 - protein, 131 aa, synthetic construct
QVQLQQSGGGLVQPGGSLRLSCAASGFTFSSYWMHWVRQAPGKGLVWVSRINSDGSSTSYADSVKGRFTISRDNAKNTLYLQMNSLRAEDTAVYYCARDRDYYGSGSYYNVGYYYYGMDVWGQGTTVTVSS

SEQ ID NO: 69 - protein, 124 aa, synthetic construct
QVQLQQSGPGLVKPSETLSLTCTVSGGSISSSSYYWGWIRQPPGKGLEWIGSIYYSGSTYYNPSLKSRVTISVDKSKNQFSLKLSSVTAADTAVYYCARHNWGRGYYYYGMDVWGQGTTVTVSS

SEQ ID NO: 70 - protein, 126 aa, synthetic construct
QVQLVESGPGLVKPSETLSLTCTVSGGSISSSSYYWGWIRQPPGKGLEWIGSIYYSGSTYYNPSLKSRVTISVDTSKNQFSLKLSSVTAADTAVYYCARLGPAAIGYSYYYGMDVWGQGTTVTVSS

SEQ ID NO: 71 - protein, 107 aa, synthetic construct
DIQMTQSPSSVSASVGDRVTITCRASQGISSWLAWYQQKPGKVPKLLIYAASSLQSGVPSRFSGSGSGTDFTLTISSLQPEDFATYYCQQANSFPLSFGGGTKVEIK

SEQ ID NO: 72 - protein, 107 aa, synthetic construct
EIVMTQSPSSVSASVGDRVTITCRASQGISSWLAWYQQKPGKAPKLLIYAASSLQSGVPSRFSGSGSGTDFTLTISSLQPEDFATYYCQQANSFPLTFGGGTKVEIK

SEQ ID NO: 73 - protein, 107 aa, synthetic construct
DVVMTQSPSSVSASVGDRVTITCRASQGISSWLAWYQQKPGKAPKLLIYAASSLQSGVPSRFSGSGSGTDFTLTISSLQPEDFAAYYCQQANSFPLTFGGGTKVEIK

SEQ ID NO: 74 - protein, 107 aa, synthetic construct
DIVMTQTPSSVSASVGDRVTITCRASQGISSWLAWYQQKPGKAPKLLIYAASSLQSGVPSRFSGSGSGTDFTLTISSLQPEDFAAYYCQQANSFPLTFGGGTKVEIK

SEQ ID NO: 75 - protein, 107 aa, synthetic construct
AIWMTQSPSSVSASVGDRVTITCRASQGISSWLAWYQQKPGKAPKLLIYAASSLQSGVPSRFSGSGSGTDFTLTISSLQPEDFATYYCQQANSFPLTFGGGTKVEIK

SEQ ID NO: 76 - protein, 107 aa, synthetic construct
DIVMTQSPSSVSASVGDRVTITCRASQGINSWLAWYQQKPGKAPKLLIYAASSLQSGVPSRFSGSGSGTDFTLTISSLQPEDFATYYCQQANSFPLTFGGGTKVEIK

SEQ ID NO: 77 - protein, 107 aa, synthetic construct
DIVMTQSPSSVSASVGDRVTITCRASQGISSWLAWYQQKPGKAPNLLIYAASSLQSGVPSRFSGSGSGTDFTLTISSLQPEDFATFYCQQANSFPLTFGGGTKVEIK

SEQ ID NO: 78 - protein, 107 aa, synthetic construct
EIVLTQSPSSVSASVGDRVTITCRASQGISSWLAWYQQKPGKAPKLLIYAASSLQSGVPSRFSGSGSGTDFTLTISSLQPEDFATYYCQQANSFPYTFGQGTKLEIK

SEQ ID NO: 79 - protein, 10 aa, synthetic construct
VVVGAGGVGK

SEQ ID NO: 80 - protein, 10 aa, synthetic construct
VVVGAVGVGK

SEQ ID NO: 81 - protein, 10 aa, synthetic construct
VVVGACGVGK

SEQ ID NO: 82 - protein, 10 aa, synthetic construct
VVVGADGVGK

SEQ ID NO: 83 - protein, 10 aa, synthetic construct
AVVGAVGVGK

SEQ ID NO: 84 - protein, 10 aa, synthetic construct
VAVGAVGVGK

SEQ ID NO: 85 - protein, 10 aa, synthetic construct
VVAGAVGVGK

SEQ ID NO: 86 - protein, 10 aa, synthetic construct
VVVAAVGVGK

SEQ ID NO: 87 - protein, 10 aa, synthetic construct
VVVGAAGVGK

SEQ ID NO: 88 - protein, 10 aa, synthetic construct
VVVGAVAVGK

SEQ ID NO: 89 - protein, 10 aa, synthetic construct
VVVGAVGAGK

SEQ ID NO: 90 - protein, 10 aa, synthetic construct
VVVGAVGVAK

SEQ ID NO: 91 - protein, 10 aa, synthetic construct
VVVGAVGVGA

SEQ ID NO: 92 - protein, 10 aa, synthetic construct
VVVGDGGVGK

SEQ ID NO: 93 - protein, 10 aa, synthetic construct
VVVGASGVGK

SEQ ID NO: 94 - protein, 10 aa, synthetic construct
IIVGAIGVGK

SEQ ID NO: 95 - protein, 10 aa, synthetic construct
VVVGDGAVGK

SEQ ID NO: 96 - protein, 10 aa, synthetic construct
KVVGARGVGK

SEQ ID NO: 97 - protein, 120 aa, synthetic construct
EVQLVESGGGLVQPGGSLRLSCAASGFNLSYSDIHWVRQAPGKGLEWVAVVMPDSGHTNYADSVKGRFTISADTSKNTAYLQMNSLRAEDTAVYYCSRATNIPVYAFDYWGQGTLVTVSS

SEQ ID NO: 98 - protein, 109 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQSYYYFRPITFGQGTKVEIK

SEQ ID NO: 99 - protein, 365 aa, Homo sapiens
MAVMAPRTLLLLLSGALALTQTWAGSHSMRYFFTSVSRPGRGEPRFIAVGYVDDTQFVRFDSDAASQRMEPRAPWIEQEGPEYWDQETRNVKAQSQTDRVDLGTLRGYYNQSEAGSHTIQIMYGCDVGSDGRFLRGYRQDAYDGKDYIALNEDLRSWTAADMAAQITKRKWEAAHEAEQLRAYLDGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 100 - protein, 365 aa, Homo sapiens
MAVMAPRTLLLLLSGALALTQTWAGSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGRCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 101 - protein, 9 aa, synthetic construct
VVGAGGVGK

SEQ ID NO: 102 - protein, 9 aa, synthetic construct
VVGAVGVGK

SEQ ID NO: 103 - protein, 124 aa, synthetic construct
EVQLVESGGGPVQAGGSLRLSCAASGRTYRGYSMGWFRQAPGREREFVAAIVWSDGNTYYEDSVKGRFTISRDNAKNTMYLQMTSLKPEDSATYYCAAKIRPYIFKIAGQYDYWGQGTQVTVSS

SEQ ID NO: 104 - protein, 107 aa, synthetic construct [sequence omitted]

SEQ ID NO: 105 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 106 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 107 - protein, 119 aa, Homo sapiens
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 108 - protein, 189 aa, synthetic construct [sequence omitted]

SEQ ID NO: 109 - protein, 365 aa, Homo sapiens [sequence omitted]

SEQ ID NO: 110 - protein, 7 aa, synthetic construct
SSSYYWG

SEQ ID NO: 111 - protein, 16 aa, synthetic construct
SIYYSGSTYYNPSLKS

SEQ ID NO: 112 - protein, 17 aa, synthetic construct
HKADNWNYAYYYYGMDV

SEQ ID NO: 113 - protein, 9 aa, synthetic construct
GGSISSSSY

SEQ ID NO: 114 - protein, 5 aa, synthetic construct
YYSGS

SEQ ID NO: 115 - protein, 17 aa, synthetic construct
HKADNWNYAYYYYGMDV

SEQ ID NO: 116 - protein, 127 aa, synthetic construct
RVQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARHKADNWNYAYYYYGMDVWGQGTTVTVSS

SEQ ID NO: 117 - protein, 107 aa, synthetic construct
DIVMTQSPSSVSASVGDRVTITCRASQGISSWLAWYQQKPGKAPKLLIYAASSLQSGVPSRFSGSGSGTDFTLTISSLQPEDFATYYCQQANSFPLTFGGGTKVEIK

SEQ ID NO: 118 - protein, 10 aa, synthetic construct
ALKASFSWAR

SEQ ID NO: 119 - protein, 10 aa, synthetic construct
ALKQSATDPR

SEQ ID NO: 120 - protein, 10 aa, synthetic construct
RIDASQLPHR

SEQ ID NO: 121 - protein, 122 aa, synthetic construct [sequence omitted]

SEQ ID NO: 122 - protein, 107 aa, synthetic construct [sequence omitted]

SEQ ID NO: 123 - protein, 104 aa, synthetic construct
ASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCD

SEQ ID NO: 124 - protein, 107 aa, synthetic construct
RTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 125 - protein, 330 aa, synthetic construct
ASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 126 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 127 - protein, 5 aa, synthetic construct
GGGGS

SEQ ID NO: 128 - protein, 15 aa, synthetic construct
GGGGSGGGGSGGGGS

(19) United States
(12) Patent Application Publication
(10) Pub. No.: US 2026/0337297 A1
(43) Pub. Date: Oct. 29, 2026

(54) ANTI-KRAS/HLA ANTIBODIES AND USES THEREOF

(71) Applicant: Biocytogen Pharmaceuticals (Beijing) Co., Ltd., Beijing (CN)

(72) Inventors: Jun Du, Beijing (CN); Wanbo Tang, Beijing (CN); Limin Zhao, Beijing (CN); Xin Jiao, Beijing (CN); Pengfei Du, Beijing (CN)

(21) Appl. No.: 19/476,488

(22) PCT Filed: Apr. 30, 2024
(86) PCT No.: PCT/CN2024/090832

§ 371 Date: Oct. 17, 2025

(30) Foreign Application Priority Data
May 6, 2023 (WO) PCT/CN2023/092478
Mar. 28, 2024 (WO) PCT/CN2024/084444

(51) Int. Cl. C07K 16/40; A61K 39/00; A61P 35/00; C07K 16/28

(52) U.S. Cl. CPC C07K 16/40; A61P 35/00; C07K 16/2809; A61K 2039/505; C07K 2317/14; C07K 2317/24; C07K 2317/31; C07K 2317/33; C07K 2317/34; C07K 2317/52; C07K 2317/565; C07K 2317/622; C07K 2317/92

(57) ABSTRACT

Provided are antibodies or antigen-binding fragments thereof that binds to a complex comprising a KRAS peptide and a MHC molecule, and the related antibody-drug conjugate (ADC) derived therefrom, chimeric antigen receptor (CAR) derived therefrom, and the uses thereof.

### CLAIM OF PRIORITY

This application claims priority to PCT/CN2023/092478, filed on May 6, 2023, and PCT/CN2024/084444, filed on Mar. 28, 2024. The entire contents of the foregoing application are incorporated herein by reference.

### TECHNICAL FIELD

This disclosure relates to antibodies or antigen-binding fragments thereof that binds to a complex comprising a KRAS peptide and a MHC molecule, and the related antibody-drug conjugate (ADC) derived therefrom, chimeric antigen receptor (CAR) derived therefrom, and the uses thereof.

### BACKGROUND

Cancer is currently one of the diseases that have the highest human mortality. According to the World Health Organization statistical data, in 2012, the number of global cancer incidence and death cases reached 14 million and 8.2 million, respectively. In China, the newly diagnosed cancer cases are 3.07 million, and the death toll is 2.2 million.

Recent clinical and commercial success of anticancer antibodies has created great interest in antibody-based therapeutics. There is a need to develop antibodies for use in various antibody-based therapeutics to treat cancers or auto-immune diseases.

### SUMMARY

This disclosure relates to anti-KRAS/HLA antibodies, antigen-binding fragment thereof, antibody-drug conjugate (ADC) derived therefrom, chimeric antigen receptor (CAR) derived therefrom, and the uses thereof. The disclosure also provides multi-specific antibodies (e.g., bispecific antibodies) that bind to an activated mutated KRAS peptide and a T cell-specific antigen (e.g., CD3).

In one aspect, the disclosure is related to an antibody or antigen-binding fragment thereof that binds to a complex comprising a KRAS (KRAS Proto-Oncogene, GTPase) peptide and a MHC molecule, comprising: a heavy chain variable region (VH) comprising complementarity determining regions (CDRs) 1, 2, and 3, in some embodiments, the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and a light chain variable region (VL) comprising CDRs 1, 2, and 3, in some embodiments, the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence, in some embodiments, the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 1, 2, 3, respectively, and the selected VL CDRs 1, 2, 3 amino acid sequences are set forth in SEQ ID NOs: 52, 53, 54, respectively;
(2) the selected VH CDRs 1, 2, 3 amino acid sequences are set forth in SEQ ID NOs: 4, 5, 6, respectively, and the selected VL CDRs 1, 2, 3 amino acid sequences are set forth in SEQ ID NOs: 55, 56, 57, respectively;
(3) the selected VH CDRs 1, 2, 3 amino acid sequences are set forth in SEQ ID NOs: 7, 8, 9, respectively, and the selected VL CDRs 1, 2, 3 amino acid sequences are set forth in SEQ ID NOs: 55, 56, 57, respectively;
(4) the selected VH CDRs 1, 2, 3 amino acid sequences are set forth in SEQ ID NOs: 10, 11, 12, respectively, and the selected VL CDRs 1, 2, 3 amino acid sequences are set forth in SEQ ID NOs: 55, 56, 57, respectively;
(5) the selected VH CDRs 1, 2, 3 amino acid sequences are set forth in SEQ ID NOs: 13, 14, 15, respectively, and the selected VL CDRs 1, 2, 3 amino acid sequences are set forth in SEQ ID NOs: 55, 56, 57, respectively;
(6) the selected VH CDRs 1, 2, 3 amino acid sequences are set forth in SEQ ID NOs: 16, 17, 18, respectively, and the selected VL CDRs 1, 2, 3 amino acid sequences are set forth in SEQ ID NOs: 55, 56, 57, respectively;
(7) the selected VH CDRs 1, 2, 3 amino acid sequences are set forth in SEQ ID NOs: 19, 20, 21, respectively, and the selected VL CDRs 1, 2, 3 amino acid sequences are set forth in SEQ ID NOs: 55, 56, 57, respectively;
(8) the selected VH CDRs 1, 2, 3 amino acid sequences are set forth in SEQ ID NOs: 22, 23, 24, respectively, and the selected VL CDRs 1, 2, 3 amino acid sequences are set forth in SEQ ID NOs: 55, 56, 57, respectively;
(9) the selected VH CDRs 1, 2, 3 amino acid sequences are set forth in SEQ ID NOs: 25, 26, 27, respectively, and the selected VL CDRs 1, 2, 3 amino acid sequences are set forth in SEQ ID NOs: 58, 59, 60, respectively;
(10) the selected VH CDRs 1, 2, 3 amino acid sequences are set forth in SEQ ID NOs: 28, 29, 30, respectively, and the selected VL CDRs 1, 2, 3 amino acid sequences are set forth in SEQ ID NOs: 52, 53, 54, respectively;
(11) the selected VH CDRs 1, 2, 3 amino acid sequences are set forth in SEQ ID NOs: 28, 29, 30, respectively, and the selected VL CDRs 1, 2, 3 amino acid sequences are set forth in SEQ ID NOs: 55, 56, 57, respectively;
(12) the selected VH CDRs 1, 2, 3 amino acid sequences are set forth in SEQ ID NOs: 31, 32, 33, respectively, and the selected VL CDRs 1, 2, 3 amino acid sequences are set forth in SEQ ID NOs: 55, 56, 57, respectively;
(13) the selected VH CDRs 1, 2, 3 amino acid sequences are set forth in SEQ ID NOs: 34, 35, 36, respectively, and the selected VL CDRs 1, 2, 3 amino acid sequences are set forth in SEQ ID NOs: 55, 56, 57, respectively;
(14) the selected VH CDRs 1, 2, 3 amino acid sequences are set forth in SEQ ID NOs: 37, 38, 39, respectively, and the selected VL CDRs 1, 2, 3 amino acid sequences are set forth in SEQ ID NOs: 55, 56, 57, respectively;
(15) the selected VH CDRs 1, 2, 3 amino acid sequences are set forth in SEQ ID NOs: 40, 41, 42, respectively, and the selected VL CDRs 1, 2, 3 amino acid sequences are set forth in SEQ ID NOs: 55, 56, 57, respectively;
(16) the selected VH CDRs 1, 2, 3 amino acid sequences are set forth in SEQ ID NOs: 43, 44, 45, respectively, and the selected VL CDRs 1, 2, 3 amino acid sequences are set forth in SEQ ID NOs: 55, 56, 57, respectively;
(17) the selected VH CDRs 1, 2, 3 amino acid sequences are set forth in SEQ ID NOs: 46, 47, 48, respectively, and the selected VL CDRs 1, 2, 3 amino acid sequences are set forth in SEQ ID NOs: 55, 56, 57, respectively;
(18) the selected VH CDRs 1, 2, 3 amino acid sequences are set forth in SEQ ID NOs: 49, 50, 51, respectively, and the selected VL CDRs 1, 2, 3 amino acid sequences are set forth in SEQ ID NOs: 58, 59, 60, respectively;
(19) the selected VH CDRs 1, 2, 3 amino acid sequences are set forth in SEQ ID NOs: 110, 111, 112, respectively, and the selected VL CDRs 1, 2, 3 amino acid sequences are set forth in SEQ ID NOs: 55, 56, 57, respectively; and
(20) the selected VH CDRs 1, 2, 3 amino acid sequences are set forth in SEQ ID NOs: 113, 114, 115, respectively, and the selected VL CDRs 1, 2, 3 amino acid sequences are set forth in SEQ ID NOs: 55, 56, 57, respectively.

In some embodiments, the VH comprises CDRs 1, 2, 3 with the amino acid sequences set forth in SEQ ID NOs: 1, 2, and 3, respectively, and the VL comprises CDRs 1, 2, 3 with the amino acid sequences set forth in SEQ ID NOs: 52, 53, and 54, respectively according to Kabat definition. In some embodiments, the VH comprises CDRs 1, 2, 3 with the amino acid sequences set forth in SEQ ID NOs: 4, 5, and 6, respectively, and the VL comprises CDRs 1, 2, 3 with the amino acid sequences set forth in SEQ ID NOs: 55, 56, and 57, respectively according to Kabat definition. In some embodiments, the VH comprises CDRs 1, 2, 3 with the amino acid sequences set forth in SEQ ID NOs: 7, 8, and 9, respectively, and the VL comprises CDRs 1, 2, 3 with the amino acid sequences set forth in SEQ ID NOs: 55, 56, and 57, respectively according to Kabat definition. In some embodiments, the VH comprises CDRs 1, 2, 3 with the amino acid sequences set forth in SEQ ID NOs: 10, 11, and 12, respectively, and the VL comprises CDRs 1, 2, 3 with the amino acid sequences set forth in SEQ ID NOs: 55, 56, and 57, respectively according to Kabat definition. In some embodiments, the VH comprises CDRs 1, 2, 3 with the amino acid sequences set forth in SEQ ID NOs: 13, 14, and 15, respectively, and the VL comprises CDRs 1, 2, 3 with the amino acid sequences set forth in SEQ ID NOs: 55, 56, and 57, respectively according to Kabat definition. In some embodiments, the VH comprises CDRs 1, 2, 3 with the amino acid sequences set forth in SEQ ID NOs: 16, 17, and 18, respectively, and the VL comprises CDRs 1, 2, 3 with the amino acid sequences set forth in SEQ ID NOs: 55, 56, and 57, respectively according to Kabat definition. In some embodiments, the VH comprises CDRs 1, 2, 3 with the amino acid sequences set forth in SEQ ID NOs: 19, 20, and 21, respectively, and the VL comprises CDRs 1, 2, 3 with the amino acid sequences set forth in SEQ ID NOs: 55, 56, and 57, respectively according to Kabat definition. In some embodiments, the VH comprises CDRs 1, 2, 3 with the amino acid sequences set forth in SEQ ID NOs: 22, 23, and 24, respectively, and the VL comprises CDRs 1, 2, 3 with the amino acid sequences set forth in SEQ ID NOs: 55, 56, and 57, respectively according to Kabat definition. In some embodiments, the VH comprises CDRs 1, 2, 3 with the amino acid sequences set forth in SEQ ID NOs: 25, 26, and 27, respectively, and the VL comprises CDRs 1, 2, 3 with the amino acid sequences set forth in SEQ ID NOs: 58, 59, and 60, respectively according to Kabat definition. In some embodiments, the VH comprises CDRs 1, 2, 3 with the amino acid sequences set forth in SEQ ID NOs: 28, 29, and 30, respectively, and the VL comprises CDRs 1, 2, 3 with the amino acid sequences set forth in SEQ ID NOs: 52, 53, and 54, respectively according to Chothia definition. In some embodiments, the VH comprises CDRs 1, 2, 3 with the amino acid sequences set forth in SEQ ID NOs: 28, 29, and 30, respectively, and the VL comprises CDRs 1, 2, 3 with the amino acid sequences set forth in SEQ ID NOs: 55, 56, and 57, respectively according to Chothia definition. In some embodiments, the VH comprises CDRs 1, 2, 3 with the amino acid sequences set forth in SEQ ID NOs: 31, 32, and 33, respectively, and the VL comprises CDRs 1, 2, 3 with the amino acid sequences set forth in SEQ ID NOs: 55, 56, and 57, respectively according to Chothia definition. In some embodiments, the VH comprises CDRs 1, 2, 3 with the amino acid sequences set forth in SEQ ID NOs: 34, 35, and 36, respectively, and the VL comprises CDRs 1, 2, 3 with the amino acid sequences set forth in SEQ ID NOs: 55, 56, and 57, respectively according to Chothia definition. In some embodiments, the VH comprises CDRs 1, 2, 3 with the amino acid sequences set forth in SEQ ID NOs: 37, 38, and 39, respectively, and the VL comprises CDRs 1, 2, 3 with the amino acid sequences set forth in SEQ ID NOs: 55, 56, and 57, respectively according to Chothia definition. In some embodiments, the VH comprises CDRs 1, 2, 3 with the amino acid sequences set forth in SEQ ID NOs: 40, 41, and 42, respectively, and the VL comprises CDRs 1, 2, 3 with the amino acid sequences set forth in SEQ ID NOs: 55, 56, and 57, respectively according to Chothia definition. In some embodiments, the VH comprises CDRs 1, 2, 3 with the amino acid sequences set forth in SEQ ID NOs: 43, 44, and 45, respectively, and the VL comprises CDRs 1, 2, 3 with the amino acid sequences set forth in SEQ ID NOs: 55, 56, and 57, respectively according to Chothia definition. In some embodiments, the VH comprises CDRs 1, 2, 3 with the amino acid sequences set forth in SEQ ID NOs: 46, 47, and 48, respectively, and the VL comprises CDRs 1, 2, 3 with the amino acid sequences set forth in SEQ ID NOs: 55, 56, and 57, respectively according to Chothia definition. In some embodiments, the VH comprises CDRs 1, 2, 3 with the amino acid sequences set forth in SEQ ID NOs: 49, 50, and 51, respectively, and the VL comprises CDRs 1, 2, 3 with the amino acid sequences set forth in SEQ ID NOs: 58, 59, and 60, respectively according to Chothia definition. In some embodiments, the VH comprises CDRs 1, 2, 3 with the amino acid sequences set forth in SEQ ID NOs: 110, 111, and 112, respectively, and the VL comprises CDRs 1, 2, 3 with the amino acid sequences set forth in SEQ ID NOs: 55, 56, and 57, respectively according to Kabat definition. In some embodiments, the VH comprises CDRs 1, 2, 3 with the amino acid sequences set forth in SEQ ID NOs: 113, 114, and 115, respectively, and the VL comprises CDRs 1, 2, 3 with the amino acid sequences set forth in SEQ ID NOs: 55, 56, and 57, respectively according to Chothia definition.

In some embodiments, the antibody or antigen-binding fragment thereof specifically binds to a complex comprising a KRAS peptide and a MHC molecule. In some embodiments, the KRAS peptide comprises a valine at a position corresponding to Gly12 of human KRAS (SEQ ID NO: 108). In some embodiments, the KRAS peptide comprises or consists of a sequence that is at least 80%, 85%, 90%, 95%, or 100% identical to the amino acid sequence of SEQ ID NO: 80. In some embodiments, the MHC is HLA (e.g., HLA-A3). In some embodiments, the antibody or antigen-binding fragment thereof is a human or humanized antibody or antigen-binding fragment thereof, a single-chain variable fragment (scFv), a one-armed antibody and/or a multi-specific antibody (e.g., a bispecific antibody). In some embodiments, the antibody or antigen-binding fragment thereof is a human IgG1 antibody or antigen-binding fragment thereof, a human IgG2 antibody or antigen-binding fragment thereof, or a human IgG4 antibody or antigen-binding fragment thereof.

In one aspect, the disclosure is related to a nucleic acid comprising a polynucleotide encoding a polypeptide comprising:
(1) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 1, 2, and 3, respectively, and in some embodiments, the VH, when paired with a light chain variable region (VL) comprising the amino acid sequence set forth in SEQ ID NO: 71 binds to a complex comprising a KRAS peptide and a MHC molecule;
(2) an immunoglobulin light chain or a fragment thereof comprising a VL comprising CDRs 1, 2, and 3 comprising the amino acid sequences set forth in SEQ ID NOs: 52, 53, and 54, respectively, and in some embodiments, the VL, when paired with a VH comprising the amino acid sequence set forth in SEQ ID NO: 61 binds to a complex comprising a KRAS peptide and a MHC molecule;
(3) an immunoglobulin heavy chain or a fragment thereof comprising a heavy chain variable region (VH) comprising CDRs 1, 2, and 3 comprising the amino acid sequences set forth in SEQ ID NOs: 4, 5, and 6, respectively, and in some embodiments, the VH, when paired with a light chain variable region (VL) comprising the amino acid sequence set forth in SEQ ID NO: 72 binds to a complex comprising a KRAS peptide and a MHC molecule;
(4) an immunoglobulin light chain or a fragment thereof comprising a VL comprising CDRs 1, 2, and 3 comprising the amino acid sequences set forth in SEQ ID NOs: 55, 56, and 57, respectively, and in some embodiments, the VL, when paired with a VH comprising the amino acid sequence set forth in SEQ ID NO: 62 binds to a complex comprising a KRAS peptide and a MHC molecule;
(5) an immunoglobulin heavy chain or a fragment thereof comprising a heavy chain variable region (VH) comprising CDRs 1, 2, and 3 comprising the amino acid sequences set forth in SEQ ID NOs: 7, 8, and 9, respectively, and in some embodiments, the VH, when paired with a light chain variable region (VL) comprising the amino acid sequence set forth in SEQ ID NO: 73 binds to a complex comprising a KRAS peptide and a MHC molecule;
(6) an immunoglobulin light chain or a fragment thereof comprising a VL comprising CDRs 1, 2, and 3 comprising the amino acid sequences set forth in SEQ ID NOs: 55, 56, and 57, respectively, and in some embodiments, the VL, when paired with a VH comprising the amino acid sequence set forth in SEQ ID NO: 63 binds to a complex comprising a KRAS peptide and a MHC molecule;
(7) an immunoglobulin heavy chain or a fragment thereof comprising a heavy chain variable region (VH) comprising CDRs 1, 2, and 3 comprising the amino acid sequences set forth in SEQ ID NOs: 7, 8, and 9, respectively, and in some embodiments, the VH, when paired with a light chain variable region (VL) comprising the amino acid sequence set forth in SEQ ID NO: 74 binds to a complex comprising a KRAS peptide and a MHC molecule;
(8) an immunoglobulin light chain or a fragment thereof comprising a VL comprising CDRs 1, 2, and 3 comprising the amino acid sequences set forth in SEQ ID NOs: 55, 56, and 57, respectively, and in some embodiments, the VL, when paired with a VH comprising the amino acid sequence set forth in SEQ ID NO: 64 binds to a complex comprising a KRAS peptide and a MHC molecule;
(9) an immunoglobulin heavy chain or a fragment thereof comprising a heavy chain variable region (VH) comprising CDRs 1, 2, and 3 comprising the amino acid sequences set forth in SEQ ID NOs: 10, 11, and 12, respectively, and in some embodiments, the VH, when paired with a light chain variable region (VL) comprising the amino acid sequence set forth in SEQ ID NO: 75 binds to a complex comprising a KRAS peptide and a MHC molecule;
(10) an immunoglobulin light chain or a fragment thereof comprising a VL comprising CDRs 1, 2, and 3 comprising the amino acid sequences set forth in SEQ ID NOs: 55, 56, and 57, respectively, and in some embodiments, the VL, when paired with a VH comprising the amino acid sequence set forth in SEQ ID NO: 65 binds to a complex comprising a KRAS peptide and a MHC molecule;
(11) an immunoglobulin heavy chain or a fragment thereof comprising a heavy chain variable region (VH) comprising CDRs 1, 2, and 3 comprising the amino acid sequences set forth in SEQ ID NOs: 13, 14, and 15, respectively, and in some embodiments, the VH, when paired with a light chain variable region (VL) comprising the amino acid sequence set forth in SEQ ID NO: 75 binds to a complex comprising a KRAS peptide and a MHC molecule;
(12) an immunoglobulin light chain or a fragment thereof comprising a VL comprising CDRs 1, 2, and 3 comprising the amino acid sequences set forth in SEQ ID NOs: 55, 56, and 57, respectively, and in some embodiments, the VL, when paired with a VH comprising the amino acid sequence set forth in SEQ ID NO: 66 binds to a complex comprising a KRAS peptide and a MHC molecule;
(13) an immunoglobulin heavy chain or a fragment thereof comprising a heavy chain variable region (VH) comprising CDRs 1, 2, and 3 comprising the amino acid sequences set forth in SEQ ID NOs: 13, 14, and 15, respectively, and in some embodiments, the VH, when paired with a light chain variable region (VL) comprising the amino acid sequence set forth in SEQ ID NO: 72 binds to a complex comprising a KRAS peptide and a MHC molecule;
(14) an immunoglobulin heavy chain or a fragment thereof comprising a heavy chain variable region (VH) comprising CDRs 1, 2, and 3 comprising the amino acid sequences set forth in SEQ ID NOs: 16, 17, and 18, respectively, and in some embodiments, the VH, when paired with a light chain variable region (VL) comprising the amino acid sequence set forth in SEQ ID NO: 75 binds to a complex comprising a KRAS peptide and a MHC molecule;
(15) an immunoglobulin light chain or a fragment thereof comprising a VL comprising CDRs 1, 2, and 3 comprising the amino acid sequences set forth in SEQ ID NOs: 55, 56, and 57, respectively, and in some embodiments, the VL, when paired with a VH comprising the amino acid sequence set forth in SEQ ID NO: 67 binds to a complex comprising a KRAS peptide and a MHC molecule;
(16) an immunoglobulin heavy chain or a fragment thereof comprising a heavy chain variable region (VH) comprising CDRs 1, 2, and 3 comprising the amino acid sequences set forth in SEQ ID NOs: 19, 20, and 21, respectively, and in some embodiments, the VH, when paired with a light chain variable region (VL) comprising the amino acid sequence set forth in SEQ ID NO: 76 binds to a complex comprising a KRAS peptide and a MHC molecule;
(17) an immunoglobulin light chain or a fragment thereof comprising a VL comprising CDRs 1, 2, and 3 comprising the amino acid sequences set forth in SEQ ID NOs: 55, 56, and 57, respectively, and in some embodiments, the VL, when paired with a VH comprising the amino acid sequence set forth in SEQ ID NO: 68 binds to a complex comprising a KRAS peptide and a MHC molecule;
(18) an immunoglobulin heavy chain or a fragment thereof comprising a heavy chain variable region (VH) comprising CDRs 1, 2, and 3 comprising the amino acid sequences set forth in SEQ ID NOs: 22, 23, and 24, respectively, and in some embodiments, the VH, when paired with a light chain variable region (VL) comprising the amino acid sequence set forth in SEQ ID NO: 77 binds to a complex comprising a KRAS peptide and a MHC molecule;
(19) an immunoglobulin light chain or a fragment thereof comprising a VL comprising CDRs 1, 2, and 3 comprising the amino acid sequences set forth in SEQ ID NOs: 55, 56, and 57, respectively, and in some embodiments, the VL, when paired with a VH comprising the amino acid sequence set forth in SEQ ID NO: 69 binds to a complex comprising a KRAS peptide and a MHC molecule;
(20) an immunoglobulin heavy chain or a fragment thereof comprising a heavy chain variable region (VH) comprising CDRs 1, 2, and 3 comprising the amino acid sequences set forth in SEQ ID NOs: 25, 26, and 27, respectively, and in some embodiments, the VH, when paired with a light chain variable region (VL) comprising the amino acid sequence set forth in SEQ ID NO: 78 binds to a complex comprising a KRAS peptide and a MHC molecule;
(21) an immunoglobulin light chain or a fragment thereof comprising a VL comprising CDRs 1, 2, and 3 comprising the amino acid sequences set forth in SEQ ID NOs: 58, 59, and 60, respectively, and in some embodiments, the VL, when paired with a VH comprising the amino acid sequence set forth in SEQ ID NO: 70 binds to a complex comprising a KRAS peptide and a MHC molecule;
(22) an immunoglobulin heavy chain or a fragment thereof comprising a heavy chain variable region (VH) comprising CDRs 1, 2, and 3 comprising the amino acid sequences set forth in SEQ ID NOs: 28, 29, and 30, respectively, and in some embodiments, the VH, when paired with a light chain variable region (VL) comprising the amino acid sequence set forth in SEQ ID NO: 71 binds to a complex comprising a KRAS peptide and a MHC molecule;
(23) an immunoglobulin heavy chain or a fragment thereof comprising a heavy chain variable region (VH) comprising CDRs 1, 2, and 3 comprising the amino acid sequences set forth in SEQ ID NOs: 28, 29, and 30, respectively, and in some embodiments, the VH, when paired with a light chain variable region (VL) comprising the amino acid sequence set forth in SEQ ID NO: 72 binds to a complex comprising a KRAS peptide and a MHC molecule;
(24) an immunoglobulin heavy chain or a fragment thereof comprising a heavy chain variable region (VH) comprising CDRs 1, 2, and 3 comprising the amino acid sequences set forth in SEQ ID NOs: 31, 32, and 33, respectively, and in some embodiments, the VH, when paired with a light chain variable region (VL) comprising the amino acid sequence set forth in SEQ ID NO: 73 binds to a complex comprising a KRAS peptide and a MHC molecule;
(25) an immunoglobulin heavy chain or a fragment thereof comprising a heavy chain variable region (VH) comprising CDRs 1, 2, and 3 comprising the amino acid sequences set forth in SEQ ID NOs: 31, 32, and 33, respectively, and in some embodiments, the VH, when paired with a light chain variable region (VL) comprising the amino acid sequence set forth in SEQ ID NO: 74 binds to a complex comprising a KRAS peptide and a MHC molecule;
(26) an immunoglobulin heavy chain or a fragment thereof comprising a heavy chain variable region (VH) comprising CDRs 1, 2, and 3 comprising the amino acid sequences set forth in SEQ ID NOs: 34, 35, and 36, respectively, and in some embodiments, the VH, when paired with a light chain variable region (VL) comprising the amino acid sequence set forth in SEQ ID NO: 75 binds to a complex comprising a KRAS peptide and a MHC molecule;
(27) an immunoglobulin heavy chain or a fragment thereof comprising a heavy chain variable region (VH) comprising CDRs 1, 2, and 3 comprising the amino acid sequences set forth in SEQ ID NOs: 37, 38, and 39, respectively, and in some embodiments, the VH, when paired with a light chain variable region (VL) comprising the amino acid sequence set forth in SEQ ID NO: 75 binds to a complex comprising a KRAS peptide and a MHC molecule;
(28) an immunoglobulin heavy chain or a fragment thereof comprising a heavy chain variable region (VH) comprising CDRs 1, 2, and 3 comprising the amino acid sequences set forth in SEQ ID NOs: 37, 38, and 39, respectively, and in some embodiments, the VH, when paired with a light chain variable region (VL) comprising the amino acid sequence set forth in SEQ ID NO: 72 binds to a complex comprising a KRAS peptide and a MHC molecule;
(29) an immunoglobulin heavy chain or a fragment thereof comprising a heavy chain variable region (VH) comprising CDRs 1, 2, and 3 comprising the amino acid sequences set forth in SEQ ID NOs: 40, 41, and 42, respectively, and in some embodiments, the VH, when paired with a light chain variable region (VL) comprising the amino acid sequence set forth in SEQ ID NO: 75 binds to a complex comprising a KRAS peptide and a MHC molecule;
(30) an immunoglobulin heavy chain or a fragment thereof comprising a heavy chain variable region (VH) comprising CDRs 1, 2, and 3 comprising the amino acid sequences set forth in SEQ ID NOs: 43, 44, and 45, respectively, and in some embodiments, the VH, when paired with a light chain variable region (VL) comprising the amino acid sequence set forth in SEQ ID NO: 76 binds to a complex comprising a KRAS peptide and a MHC molecule;
(31) an immunoglobulin heavy chain or a fragment thereof comprising a heavy chain variable region (VH) comprising CDRs 1, 2, and 3 comprising the amino acid sequences set forth in SEQ ID NOs: 46, 47, and 48, respectively, and in some embodiments, the VH, when paired with a light chain variable region (VL) comprising the amino acid sequence set forth in SEQ ID NO: 77 binds to a complex comprising a KRAS peptide and a MHC molecule;
(32) an immunoglobulin heavy chain or a fragment thereof comprising a heavy chain variable region (VH) comprising CDRs 1, 2, and 3 comprising the amino acid sequences set forth in SEQ ID NOs: 49, 50, and 51, respectively, and in some embodiments, the VH, when paired with a light chain variable region (VL) comprising the amino acid sequence set forth in SEQ ID NO: 78 binds to a complex comprising a KRAS peptide and a MHC molecule;
(33) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 110, 111, and 112, respectively, and wherein the VH, when paired with a light chain variable region (VL) comprising the amino acid sequence set forth in SEQ ID NO: 117 binds to a complex comprising a KRAS peptide and a MHC molecule;
(34) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 113, 114, and 115, respectively, and wherein the VH, when paired with a light chain variable region (VL) comprising the amino acid sequence set forth in SEQ ID NO: 117 binds to a complex comprising a KRAS peptide and a MHC molecule; or
(3) an immunoglobulin light chain or a fragment thereof comprising a VL comprising CDRs 1, 2, and 3 comprising the amino acid sequences set forth in SEQ ID NOs: 55, 56, and 57, respectively, and wherein the VL, when paired with a VH comprising the amino acid sequence set forth in SEQ ID NO: 116 binds to a complex comprising a KRAS peptide and a MHC molecule.

In some embodiments, the VH when paired with a VL specifically binds to a complex comprising a KRAS peptide and a MHC molecule, or the VL when paired with a VH specifically binds to a complex comprising a KRAS peptide and a MHC molecule. In some embodiments, the immunoglobulin heavy chain or the fragment thereof is a human immunoglobulin heavy chain or a fragment thereof (e.g., a human IgG1 heavy chain or a fragment thereof, a human IgG2 heavy chain or a fragment thereof, or a human IgG4 heavy chain or a fragment thereof), and the immunoglobulin light chain or the fragment thereof is a human immunoglobulin light chain or a fragment thereof. In some embodiments, the nucleic acid encodes a single-chain variable fragment (scFv), a one-armed antibody, a multi-specific antibody (e.g., a bispecific antibody), or a chimeric antigen receptor (CAR). In some embodiments, the nucleic acid is cDNA.

In one aspect, the disclosure is related to a vector comprising one or more of the nucleic acids described herein. In one aspect, the disclosure is related to a vector comprising two of the nucleic acids described herein, in some embodiments, the vector encodes the VH region and the VL region that together bind to a complex comprising a KRAS peptide and a MHC molecule. In one aspect, the disclosure is related to a pair of vectors, in some embodiments, each vector comprises one of the nucleic acids described herein, in some embodiments, together the pair of vectors encodes the VH region and the VL region that together bind to a complex comprising a KRAS peptide and a MHC molecule.

In one aspect, the disclosure is related to a cell comprising the vector or the pair of vectors described herein. In some embodiments, the cell is a CHO cell. In one aspect, the disclosure is related to a cell comprising one or more of the nucleic acids described herein. In one aspect, the disclosure is related to a cell comprising two of the nucleic acids described herein. In some embodiments, the two nucleic acids together encode the VH region and the VL region that together bind to a complex comprising a KRAS peptide and a MHC molecule.

In one aspect, the disclosure is related to a method of producing an antibody or an antigen-binding fragment thereof, the method comprising (a) culturing the cell described herein under conditions sufficient for the cell to produce the antibody or the antigen-binding fragment; and (b) collecting the antibody or the antigen-binding fragment produced by the cell.

In one aspect, the disclosure is related to an antibody or antigen-binding fragment thereof that binds to a complex comprising a KRAS peptide and a MHC molecule, comprising a heavy chain variable region (VH) comprising an amino acid sequence that is at least 80% identical to a selected VH sequence, and a light chain variable region (VL) comprising an amino acid sequence that is at least 80% identical to a selected VL sequence, in some embodiments, the selected VH sequence and the selected VL sequence are one of the following: (1) the selected VH sequence is SEQ ID NO: 61, and the selected VL sequence is SEQ ID NO: 71; (2) the selected VH sequence is SEQ ID NO: 62, and the selected VL sequence is SEQ ID NO: 72; (3) the selected VH sequence is SEQ ID NO: 63, and the selected VL sequence is SEQ ID NO: 73; (4) the selected VH sequence is SEQ ID NO: 64, and the selected VL sequence is SEQ ID NO: 74; (5) the selected VH sequence is SEQ ID NO: 65, and the selected VL sequence is SEQ ID NO: 75; (6) the selected VH sequence is SEQ ID NO: 66, and the selected VL sequence is SEQ ID NO: 75; (7) the selected VH sequence is SEQ ID NO: 66, and the selected VL sequence is SEQ ID NO: 72; (8) the selected VH sequence is SEQ ID NO: 67, and the selected VL sequence is SEQ ID NO: 75; (9) the selected VH sequence is SEQ ID NO: 68, and the selected VL sequence is SEQ ID NO: 76; (10) the selected VH sequence is SEQ ID NO: 69, and the selected VL sequence is SEQ ID NO: 77; (11) the selected VH sequence is SEQ ID NO: 70, and the selected VL sequence is SEQ ID NO: 78; and (12) the selected VH sequence is SEQ ID NO: 116, and the selected VL sequence is SEQ ID NO: 117.

In some embodiments, the VH comprises the sequence of SEQ ID NO: 61 and the VL comprises the sequence of SEQ ID NO: 71. In some embodiments, the VH comprises the sequence of SEQ ID NO: 62 and the VL comprises the sequence of SEQ ID NO: 72. In some embodiments, the VH comprises the sequence of SEQ ID NO: 63 and the VL comprises the sequence of SEQ ID NO: 73. In some embodiments, the VH comprises the sequence of SEQ ID NO: 64 and the VL comprises the sequence of SEQ ID NO: 74. In some embodiments, the VH comprises the sequence of SEQ ID NO: 65 and the VL comprises the sequence of SEQ ID NO: 75. In some embodiments, the VH comprises the sequence of SEQ ID NO: 66 and the VL comprises the sequence of SEQ ID NO: 75. In some embodiments, the VH comprises the sequence of SEQ ID NO: 66 and the VL comprises the sequence of SEQ ID NO: 72. In some embodiments, the VH comprises the sequence of SEQ ID NO: 67 and the VL comprises the sequence of SEQ ID NO: 75. In some embodiments, the VH comprises the sequence of SEQ ID NO: 68 and the VL comprises the sequence of SEQ ID NO: 76. In some embodiments, the VH comprises the sequence of SEQ ID NO: 69 and the VL comprises the sequence of SEQ ID NO: 77. In some embodiments, the VH comprises the sequence of SEQ ID NO: 70 and the VL comprises the sequence of SEQ ID NO: 78. In some embodiments, the VH comprises the sequence of SEQ ID NO: 116 and the VL comprises the sequence of SEQ ID NO: 117.

In one aspect, the disclosure is related to an antibody or antigen-binding fragment thereof that binds to a complex comprising a KRAS peptide and a MHC molecule, comprising a heavy chain variable region (VH) comprising VH CDR1, VH CDR2, and VH CDR3 that are identical to VH CDR1, VH CDR2, and VH CDR3 of a selected VH sequence; and a light chain variable region (VL) comprising VL CDR1, VL CDR2, and VL CDR3 that are identical to VL CDR1, VL CDR2, and VL CDR3 of a selected VL sequence, in some embodiments, the selected VH sequence and the selected VL sequence are one of the following: (1) the selected VH sequence is SEQ ID NO: 61, and the selected VL sequence is SEQ ID NO: 71; (2) the selected VH sequence is SEQ ID NO: 62, and the selected VL sequence is SEQ ID NO: 72; (3) the selected VH sequence is SEQ ID NO: 63, and the selected VL sequence is SEQ ID NO: 73; (4) the selected VH sequence is SEQ ID NO: 64, and the selected VL sequence is SEQ ID NO: 74; (5) the selected VH sequence is SEQ ID NO: 65, and the selected VL sequence is SEQ ID NO: 75; (6) the selected VH sequence is SEQ ID NO: 66, and the selected VL sequence is SEQ ID NO: 75; (7) the selected VH sequence is SEQ ID NO: 66, and the selected VL sequence is SEQ ID NO: 72; (8) the selected VH sequence is SEQ ID NO: 67, and the selected VL sequence is SEQ ID NO: 75; (9) the selected VH sequence is SEQ ID NO: 68, and the selected VL sequence is SEQ ID NO: 76; (10) the selected VH sequence is SEQ ID NO: 69, and the selected VL sequence is SEQ ID NO: 77; (11) the selected VH sequence is SEQ ID NO: 70, and the selected VL sequence is SEQ ID NO: 78; and (12) the selected VH sequence is SEQ ID NO: 116, and the selected VL sequence is SEQ ID NO: 117.

In some embodiments, the antibody or antigen-binding fragment specifically binds to a complex comprising a KRAS peptide and a MHC molecule. In some embodiments, the KRAS peptide comprises a valine at a position corresponding to Gly12 of human KRAS (SEQ ID NO: 108). In some embodiments, the KRAS peptide comprises or consists of a sequence that is at least 80%, 85%, 90%, 95%, or 100% identical to the amino acid sequence of SEQ ID NO: 80. In some embodiments, the MHC is HLA (e.g., HLA-A3). In some embodiments, the antibody or antigen-binding fragment thereof is a human or humanized antibody or antigen-binding fragment thereof, a single-chain variable fragment (scFv), a one-armed antibody and/or a multi-specific antibody (e.g., a bispecific antibody). In some embodiments, the antibody or antigen-binding fragment thereof is a human IgG1 antibody or antigen-binding fragment thereof, a human IgG2 antibody or antigen-binding fragment thereof, or a human IgG4 antibody or antigen-binding fragment thereof.

In one aspect, the disclosure is related to an antibody or antigen-binding fragment thereof that cross-competes with the antibody or antigen-binding fragment thereof described herein.

In some embodiments, the antibody or antigen-binding fragment thereof comprises a fragment crystallizable region (Fc region).

In one aspect, the disclosure is related to a protein construct that binds to a complex comprising a KRAS peptide and a MHC molecule, comprising: (1) a first functional moiety comprising an antibody or antigen-binding fragment thereof described herein; and (2) a second functional moiety comprising a T-cell engaging molecule. In some embodiments, the T-cell engaging molecule (e.g., VHH or scFv) targets human CD3. In some embodiments, the KRAS peptide comprises or consists of a sequence that is at least 80%, 85%, 90%, 95%, or 100% identical to the amino acid sequence of SEQ ID NO: 80. In some embodiments, the MHC is HLA (e.g., HLA-A3). In some embodiments, the first functional moiety and the second functional moiety are connected via a linker.

In one aspect, the disclosure is related to a protein construct, comprising: (1) a first functional moiety comprising an antibody or antigen-binding fragment thereof described herein; (2) a second functional moiety comprising a T-cell engaging molecule; and (3) a third functional moiety comprising a single chain human crystallizable fragment. In some embodiments, the T-cell engaging molecule is a scFv or VHH targeting human CD3. In some embodiments, the first functional moiety, the second functional moiety, and the third functional moiety are connected via one or more linkers. In some embodiments, the KRAS peptide comprises or consists of a sequence that is at least 80%, 85%, 90%, 95%, or 100% identical to the amino acid sequence of SEQ ID NO: 80. In some embodiments, the MHC is HLA (e.g., HLA-A3).

In one aspect, the disclosure is related to an antibody-drug conjugate comprising the antibody or antigen-binding fragment thereof, or the protein construct described herein covalently bound to a therapeutic agent. In some embodiments, the therapeutic agent is a cytotoxic or cytostatic agent. In some embodiments, the drug-to-antibody ratio (DAR) is about 4.

In one aspect, the disclosure is related to an engineered receptor comprising the antibody or antigen-binding fragment thereof described herein. In some embodiments, the engineered receptor further comprises a transmembrane region, and an intracellular signaling domain. In some embodiments, the engineered receptor is a chimeric antigen receptor (“CAR”). In some embodiments, the engineered receptor further comprises a hinge region. In some embodiments, the transmembrane region comprises a transmembrane region of CD4, CD8, and/or CD28, or a portion thereof. In some embodiments, the intracellular signaling domain comprises a primary intracellular signaling sequence of an immune effector cell. In some embodiments, the intracellular signaling domain is or comprises a functional signaling domain of CD3 zeta. In some embodiments, the intracellular signaling domain further comprises a costimulatory signaling domain. In some embodiments, the costimulatory signaling domain comprises a functional signaling domain from a protein selected from the group consisting of a MHC class I molecule, a TNF receptor protein, an Immunoglobulin-like protein, a cytokine receptor, an integrin, a signaling lymphocytic activation molecule (SLAM protein), an activating NK cell receptor, BTLA, a Toll ligand receptor, OX40, CD2, CD7, CD27, CD28, CD30, CD40, CDS, ICAM-1, LFA-1, CD11a/CD18, 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, LA4, CD49D, ITGA6, VLA-6, CD49f, ITGAD, CD11d, ITGAE, CD103, ITGAL, CD11a, LFA-1, ITGAM, CD 11b, ITGAX, CD 11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a CD83 ligand. In some embodiments, the costimulatory signaling domain comprises an intracellular signaling domain of 4-1BB and/or CD28. In some embodiments, the engineered receptor comprises a signal peptide.

In some embodiments, the engineered receptor is a chimeric T cell receptor (chimeric TCR or “cTCR”). In some embodiments, the cTCR comprises an alpha chain comprising a variable alpha (Va) region and a beta chain comprising a variable beta (Vb) region, in some embodiments, the Va region is derived from the VH of the antibody or antigen-binding fragment thereof, and the Vb region is derived from the VL of the antibody or antigen-binding fragment thereof. In some embodiments, the alpha chain further comprises an alpha chain constant region, and the beta chain further comprises a beta chain constant region. In some embodiments, the cTCR further comprises a transmembrane region, and a short cytoplasmic tail. In some embodiments, the cTCR is further associated with CD3.

In one aspect, the disclosure is related to a polynucleotide encoding the engineered receptor described herein.

In one aspect, the disclosure is related to a vector comprising the polynucleotide described herein. In some embodiments, the vector is a viral vector.

In one aspect, the disclosure is related to an engineered cell expressing the engineered receptor described herein. In some embodiments, the engineered cell is an immune cell. In some embodiments, the immune cell is an NK cell or a T cell. In some embodiments, the engineered cell is a T cell. In some embodiments, the T cell is selected from the group consisting of cytotoxic T cell, a helper T cell, a natural killer T (NK-T) cell, and a γδT cell.

In one aspect, the disclosure is related to a method for producing an engineered cell, comprising introducing a vector described herein into a cell in vitro or ex vivo. In some embodiments, the vector is a viral vector and the introducing is carried out by transduction.

In one aspect, the disclosure is related to a method of treating a subject having cancer, the method comprising administering a therapeutically effective amount of a composition comprising the antibody or antigen-binding fragment thereof, the protein construct, the antibody-drug conjugate, or the engineered cell described herein, to the subject. In some embodiments, the cancer comprises one or more cancer cells that express KRAS G12V. In some embodiments, the subject has a solid tumor. In some embodiments, the cancer is pancreatic cancer, colorectal cancer, non-small cell lung cancer (NSCLC), ovarian cancer, esophageal cancer, or bile duct cancer. In some embodiments, the method described herein further comprises administering a therapeutically effective amount of an anti-OX40 antibody, an anti-PD1 antibody, an anti-PDL1 antibody, an anti-PDL2 antibody, an anti-LAG-3 antibody, an anti-TIGIT antibody, an anti-CTLA-4 antibody, an anti-GITR antibody, an anti-TIM-3 antibody, an anti-4-1BB antibody, and/or an anti-CD40 antibody, to the subject.

In one aspect, the disclosure is related to a method of decreasing the rate of tumor growth, the method comprising contacting a tumor cell with an effective amount of a composition comprising the antibody or antigen-binding fragment thereof, the protein construct, the antibody-drug conjugate, or the engineered cell described herein.

In one aspect, the disclosure is related to a method of killing a tumor cell, the method comprising contacting a tumor cell with an effective amount of a composition comprising the antibody or antigen-binding fragment thereof, the protein construct, the antibody-drug conjugate, or the engineered cell described herein.

In one aspect, the disclosure is related to a method of increasing immune response in a subject, the method comprising administering to the subject an effective amount of a composition comprising the antibody or antigen-binding fragment thereof, the protein construct, the antibody-drug conjugate, or the engineered cell described herein.

In one aspect, the disclosure is related to a pharmaceutical composition comprising the antibody or antigen-binding fragment thereof, the protein construct, the antibody-drug conjugate, or the engineered cell described herein, and a pharmaceutically acceptable carrier.

In one aspect, the disclosure is related to an antibody or antigen-binding fragment thereof that binds to a complex comprising a KRAS peptide and a MHC molecule, in some embodiments, the antibody or antigen-binding fragment thereof binds specifically to an epitope in the KRAS peptide, in some embodiments, the epitope is an amino acid residue corresponding to Val6 of SEQ ID NO: 80.

As used herein, the term “cancer” refers to cells having the capacity for autonomous growth. Examples of such cells include cells having an abnormal state or condition characterized by rapidly proliferating cell growth. The term is meant to include cancerous growths, e.g., tumors; oncogenic processes, metastatic tissues, and malignantly transformed cells, tissues, or organs, irrespective of histopathologic type or stage of invasiveness. Also included are malignancies of the various organ systems, such as head and neck, respiratory, cardiovascular, renal, reproductive, hematological, neurological, hepatic, gastrointestinal, and endocrine systems; as well as adenocarcinomas which include malignancies such as most colon cancers, renal-cell carcinoma, prostate cancer and/or testicular tumors, non-small cell carcinoma of the lung, glioma and cancer of the small intestine. Cancer that is “naturally arising” includes any cancer that is not experimentally induced by implantation of cancer cells into a subject, and includes, for example, spontaneously arising cancer, cancer caused by exposure of a patient to a carcinogen(s), cancer resulting from insertion of a transgenic oncogene or knockout of a tumor suppressor gene, and cancer caused by infections, e.g., viral infections. The term “carcinoma” is art recognized and refers to malignancies of epithelial or endocrine tissues. The term also includes carcinosarcomas, which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation. The term “hematopoietic neoplastic disorders” includes diseases involving hyperplastic/neoplastic cells of hematopoietic origin. A hematopoietic neoplastic disorder can arise from myeloid, lymphoid or erythroid lineages, or precursor cells thereof.

As used herein, the term “antibody” refers to any antigen-binding molecule that contains at least one (e.g., one, two, three, four, five, or six) complementary determining region (CDR) (e.g., any of the three CDRs from an immunoglobulin light chain or any of the three CDRs from an immunoglobulin heavy chain) and is capable of specifically binding to an epitope. Non-limiting examples of antibodies include: monoclonal antibodies, polyclonal antibodies, multi-specific antibodies (e.g., bi-specific antibodies), single-chain antibodies, single variable domain (VHH) antibodies, chimeric antibodies, human antibodies, and humanized antibodies. In some embodiments, an antibody can contain an Fc region of a human antibody. The term antibody also includes derivatives, e.g., bi-specific antibodies, single-chain antibodies, diabodies, linear antibodies, and multi-specific antibodies formed from antibody fragments.

As used herein, the term “antigen-binding fragment” refers to a portion of a full-length antibody, wherein the portion of the antibody is capable of specifically binding to an antigen. In some embodiments, the antigen-binding fragment contains at least one variable domain (e.g., a variable domain of a heavy chain or a variable domain of light chain). Non-limiting examples of antibody fragments include, e.g., Fab, Fab′, F(ab′)₂, and Fv fragments.

As used herein, the term “anti-KRAS/HLA antibody” refers to an antibody that can specifically binds to a MHC complex comprising a KRAS peptide and a HLA molecule (e.g., HLA-A3 or HLA-A11).

As used herein, the term “human antibody” refers to an antibody that is encoded by an endogenous nucleic acid (e.g., rearranged human immunoglobulin heavy or light chain locus) derived from a human. In some embodiments, a human antibody is collected from a human or produced in a human cell culture (e.g., human hybridoma cells). In some embodiments, a human antibody is produced in a non-human cell (e.g., a mouse or hamster cell line). In some embodiments, a human antibody is produced in a bacterial or yeast cell. In some embodiments, a human antibody is produced in a transgenic non-human animal (e.g., a bovine) containing an unrearranged or rearranged human immunoglobulin locus (e.g., heavy or light chain human immunoglobulin locus).

As used herein, the term “chimeric antibody” refers to an antibody that contains a sequence present in at least two different species (e.g., antibodies from two different mammalian species such as a human and a mouse antibody). A non-limiting example of a chimeric antibody is an antibody containing the variable domain sequences (e.g., all or part of a light chain and/or heavy chain variable domain sequence) of a non-human (e.g., mouse) antibody and the constant domains of a human antibody. Additional examples of chimeric antibodies are described herein and are known in the art.

As used herein, the term “humanized antibody” refers to a non-human antibody which contains minimal sequence derived from a non-human (e.g., mouse) immunoglobulin and contains sequences derived from a human immunoglobulin. In non-limiting examples, humanized antibodies are human antibodies (recipient antibody) in which hypervariable (e.g., CDR) region residues of the recipient antibody are replaced by hypervariable (e.g., CDR) region residues from a non-human antibody (e.g., a donor antibody), e.g., a mouse, rat, or rabbit antibody, having the desired specificity, affinity, and capacity. In some embodiments, the Fv framework residues of the human immunoglobulin are replaced by corresponding non-human (e.g., mouse) immunoglobulin residues. In some embodiments, humanized antibodies may contain residues which are not found in the recipient antibody or in the donor antibody. These modifications can be made to further refine antibody performance. In some embodiments, the humanized antibody contains substantially all of at least one, and typically two, variable domains, in which all or substantially all of the hypervariable loops (CDRs) correspond to those of a non-human (e.g., mouse) immunoglobulin and all or substantially all of the framework regions are those of a human immunoglobulin. The humanized antibody can also contain at least a portion of an immunoglobulin constant region (Fc), typically, that of a human immunoglobulin. Humanized antibodies can be produced using molecular biology methods known in the art. Non-limiting examples of methods for generating humanized antibodies are described herein.

As used herein, the term “single-chain antibody” refers to a single polypeptide that contains at least two immunoglobulin variable domains (e.g., a variable domain of a mammalian immunoglobulin heavy chain or light chain) that is capable of specifically binding to an antigen. Non-limiting examples of single-chain antibodies are described herein.

As used herein, the terms “subject” and “patient” are used interchangeably throughout the specification and describe an animal, human or non-human, to whom treatment according to the methods of the present invention is provided. Veterinary and non-veterinary applications are contemplated by the present disclosure. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

As used herein, when referring to an antibody, the phrases “specifically binding” and “specifically binds” mean that the antibody interacts with its target molecule preferably to other molecules, because the interaction is dependent upon the presence of a particular structure (i.e., the antigenic determinant or epitope) on the target molecule; in other words, the reagent is recognizing and binding to molecules that include a specific structure rather than to all molecules in general. An antibody that specifically binds to the target molecule may be referred to as a target-specific antibody. For example, an antibody that specifically binds to a KRAS/HLA complex may be referred to as a KRAS/HLA-specific antibody, an anti-KRAS/HLA antibody, or an anti-KARS/HLA complex antibody. In some embodiments, the anti-KRAS/HLA antibody can also be referred to as an anti-KRAS G12V/HLA antibody.

As used herein, the term “bispecific antibody” refers to an antibody that binds to two different epitopes. The epitopes can be on the same antigen or on different antigens.

As used herein, the term “multispecific antibody” refers to an antibody that binds to two or more different epitopes. The epitopes can be on the same antigen or on different antigens. A multispecific antibody can be e.g., a bispecific antibody or a trispecific antibody. In some embodiments, the multispecific antibody binds to two, three, four, five, or six different epitopes.

As used herein, a “chimeric antigen receptor” or “CAR” refers to a fusion protein comprising an extracellular domain capable of binding to an antigen, and an intracellular region comprising one or more intracellular signaling domains derived from signal transducing proteins. The extracellular domain can be any proteinaceous molecule or part thereof that can specifically bind to a predetermined antigen. In some embodiments, the extracellular domain comprises an antibody or antigen binding fragment thereof. In some embodiments, the intracellular signaling domain can be any oligopeptide or polypeptide domain known to function to transmit a signal causing activation or inhibition of a biological process in a cell, for example, activation of an immune cell such as a T cell or a NK cell.

As used herein, the terms “polypeptide,” “peptide,” and “protein” are used interchangeably to refer to polymers of amino acids of any length of at least two amino acids.

As used herein, the terms “polynucleotide,” “nucleic acid molecule,” and “nucleic acid sequence” are used interchangeably herein to refer to polymers of nucleotides of any length of at least two nucleotides, and include, without limitation, DNA, RNA, DNA/RNA hybrids, and modifications thereof.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-1D show schematic structures of exemplary anti-KRAS/CD3 bispecific antibody.

FIG. 2 lists Kabat CDR sequences for anti-KRAS/HLA antibody heavy chains.

FIG. 3 lists Chothia CDR sequences for anti-KRAS/HLA antibody heavy chains.

FIG. 4 lists Kabat and Chothia CDR sequences for anti-KRAS/HLA antibody light chains.

FIG. 5 lists amino acid sequences discussed in the disclosure.

FIGS. 6A-6D show binding curves of anti-KRAS/HLA antibodies 1B6-SI (FIG. 6A), 1C10-SI (FIG. 6B), 1H2-SI (FIG. 6C), and 2B12-SI (FIG. 6D), respectively, binding to COS-7-HLA-A03 cells pulsed with KRAS G12V (7-16) peptide, KRAS G12WT (7-16) peptide, KRAS G12C (7-16) peptide, or KRAS G12D (7-16) peptide. COS-7-HLA-A03 cells not pulsed with peptides were used as a control.

FIGS. 6E-6F show binding curves of anti-KRAS/HLA antibodies P03141-SI (FIG. 6E) and V2-SI (FIG. 6F), respectively, binding to COS-7-HLA-A03 cells pulsed with KRAS G12V (7-16) peptide or KRAS G12WT (7-16) peptide. COS-7-HLA-A03 cells not pulsed with peptides were used as a control.

FIG. 7 shows the binding affinities of anti-KRAS/HLA antibodies V2-SI, 1G10-SI, 1H2-SI, 2B12-SI, 1F3-SI, 1G1-SI, 1A7-SI, 1B6-SI, 1C10-SI, and 1E12-SI, respectively, to COS-7-HLA-A03 cells pulsed with KRAS G12V peptide, or COS-7-HLA-A1101 cells pulsed with KRAS G12V (7-16) peptide, KRAS G12V (8-16) peptide, KRAS G12WT (7-16) peptide, or KRAS G12WT (8-16) peptide. ISO is an antibody isotype control.

FIG. 8A shows the binding affinities of anti-KRAS/HLA antibodies V2-SI, 1H2-SI, 2B12-SI, 1G1-SI, 1B6-SI, 1C10-SI, 1F3-SI, 1A7-SI, 1G10-SI, 1E12-SI, and 1F9-SI, respectively, to COS-7-HLA-A03 cells incubated with MRAS peptide, ERAS peptide, Rab-7b peptide, RhoJ peptide, or mRho GTPase2 peptide. COS-7-HLA-A03 cells not pulsed with peptides were used as a control. ISO is an antibody isotype control.

FIG. 8B shows the binding affinity of anti-KRAS/HLA antibody P03141-SI to COS-7-HLA-A03 cells incubated with MRAS peptide, Rab-7b peptide, RhoJ peptide, or mRho GTPase2 peptide. COS-7-HLA-A03 cells not pulsed with peptides were used as a control.

FIGS. 9A-9L show the cell lysis percentage (%) of Raji cells expressing wild-type KRAS or CFPAC-1 cells expressing KRAS G12V that were co-cultured with CD3+ T cells, in the presence of anti-KRAS/CD3 bispecific antibodies V2-CD3 (FIG. 9A), 1A7-CD3 (FIG. 9B), 1B6-CD3 (FIG. 9C), 1C10-CD3 (FIG. 9D), 1E12-CD3 (FIG. 9E), 1F1-CD3 (FIG. 9F), 1F3-CD3 (FIG. 9G), 1F9-CD3 (FIG. 9H), 1G1-CD3 (FIG. 9I), 1G10-CD3 (FIG. 9J), 1H2-CD3 (FIG. 9K), and 2B12-CD3 (FIG. 9L), respectively.

FIGS. 10A-10E show the binding between COS-7-HLA-A03 cells pulsed with alanine-replaced peptides (50 μM) and the anti-KRAS/HLA antibodies, including 1B6-SI (FIG. 10A), 1G1-SI (FIG. 10B), 1H2-SI (FIG. 10C), 2B12-SI (FIG. 10D), and P03141-SI (FIG. 10E), as determined by flow cytometry.

FIG. 11 shows the cell lysis percentage (%) of RKO cells, CFPAC-1 cells or NCI-H441 cells that were co-cultured with CD3+ T cells, in the presence of anti-KRAS/CD3 bispecific antibodies P03141-CD3-SRY(A), P03141-CD3 (B), 1H2-CD3-SRY (C), and 1H2-CD3 (D), respectively.

FIG. 12 shows the percentage of CD137+ T cells (%) that were co-cultured with RKO cells, CFPAC-1 cells or NCI-H441 cells, in the presence of anti-KRAS/CD3 bispecific antibodies P03141-CD3-SRY(A), P03141-CD3 (B), 1H2-CD3-SRY (C), and 1H2-CD3 (D) respectively, as determined by flow cytometry.

FIG. 13 shows the cytokine IFN-γ release at 72 h of CD3+ T cells that were co-cultured with RKO cells, CFPAC-1 cells or NCI-H441 cells, in the presence of anti-KRAS/CD3 bispecific antibodies P03141-CD3-SRY(A), P03141-CD3 (B), 1H2-CD3-SRY (C), and 1H2-CD3 (D), respectively, as determined by flow cytometry.

FIGS. 14A-14B show the killing of SW620 tumor cells in the presence of anti-KRAS/CD3 bispecific antibodies P03141-CD3-SLE (FIG. 14A) or P03141-CD3-ScDb (FIG. 14B), as determined by IncuCyte.

FIG. 15 shows binding curves of anti-KRAS/HLA antibody P03141-CD3-SLE to modified Jarkat cells.

FIG. 16 shows the binding between COS-7-HLA-A03 cells pulsed with amino acid-replaced peptides (50 μM) and the anti-KRAS/HLA antibody P03141-SI, as determined by flow cytometry.

FIG. 17 shows the binding affinity of anti-KRAS/HLA antibody P03141-SI to COS-7-HLA-A03 cells incubated with FNDC7 peptide, 3IS57 peptide or SMIM2 peptide. COS-7-HLA-A03 cells not pulsed with peptides were used as a control. ISO is an antibody isotype control.

### DETAILED DESCRIPTION

Global cancer incidence is on the rise, and the global cancer burden is expected to reach 28.4 million cases in 2040, a 47% rise from 2020. KRAS is one of the most frequently mutated oncogenes in all human malignancies and is seen in 1 in 7 of all human cancers. KRAS is present and expressed in all human cells as a membrane bound protein.

Mutations in Kirsten rat sarcoma viral oncogene homolog (KRAS) are one of the most common oncogenic events in endodermal carcinomas. The KRAS gene can simultaneously harbor multiple mutations that can potentiate tumor-promoting activity. In fact, alterations in KRAS have been identified in 25% of all cancers, where some cancers like pancreatic cancer contain extremely high mutation rates (90%), while others, such as prostate cancer, show lower mutation rates (7%). Not only do KRAS mutations promote and maintain tumorigenesis, they also increase the chance of resistance and poor prognosis, ultimately contributing to over one million deaths annually. Even though mutations in other RAS isoforms, including the neuroblastoma RAS viral (v-ras) oncogene homolog (NRAS) and Harvey rat sarcoma viral oncogene homolog (HRAS) are prevalent in many cancer types, mutations in KRAS account for 85% of all RAS isoform mutations. Thus, anti-KRAS antibodies can be potentially used as cancer therapies.

The present disclosure provides examples of antibodies, antigen-binding fragment thereof, that bind to a MHC complex comprising a KRAS peptide (e.g., with G12V mutation).

### KRAS and its Oncogenic Mutations

KRAS has been identified as a KRAS-1 pseudogene on short arm of chromosome 6 and KRAS-2 gene, located on the short arm of chromosome 12 (12p11.1-12p12.1). KRAS-2 coding region spans across six exons and measures over 45 kB. The two protein isoforms of KRAS-2, KRAS-4A and KRAS-4B are produced due to alternative splicing on its fourth exon, leading to 188 and 189 monomeric amino acid sequences, respectively. For clinical and research purposes, the term KRAS refers to KRAS-4B, which constitutes the major transcriptomic product in human cells. RAS proteins belong to the super family of small GTPases and bind exclusively to GTP (G proteins). KRAS protein product consists of 2 domains, the N-terminal catalytic (guanine binding) domain (G-domain) and the hypervariable region (HVR) at the C-terminal. The catalytic domain is a highly conserved region with a high degree of homology. It consists of the P-loop, switch I, and switch II regions. The G-domain facilitates GTP-GDP exchange and functions as a GTP-GDP switch. The P-loop is the phosphate binding region and stabilizes the nucleotide phosphates while the switch regions form binding surfaces for effector proteins. This G-domain switch is regulated primarily by guanine exchange factors (GEFs) that promote GDP to GTP switch and activation, and deactivating factors such as GTPase activating proteins (GAPs).

The C-domain has a high degree of variability and is responsible for anchorage of RAS to the inner surface of plasma membrane. It includes the CAAX box (cysteine, 2 aliphatic amino acids, another residue) and is responsible for post-translation modifications such as prenylation. Prenylation is a process by which a farnesyl or geranylgeranyl moiety is added to the terminal cysteine of the CAAX box by farnesyltransferase (FTase) or geranylgeranyltransferase (GGTase). This is followed by cleavage of the AAX residues and methylation of the cysteine residue by isoprenylcysteine methyltransferase (ICMT). KRAS monomers require localization to the cell membrane for their activity. More recent evidence suggests that KRAS monomers undergo dimerization for their downstream signaling activity.

KRAS activation can occur because of several upstream signals such as growth factors like epidermal growth factor (EGF), platelet derived growth factor (PDGF), insulin-like growth factor (IGF), and fibroblast growth factor (FGF), receptor tyrosine kinase (RTK) activation, and cytokines. KRAS activation consists of phosphorylation of GDP-bound KRAS to a GTP-bound state with the assistance of RAS GEFs.

KRAS activation leads to downstream signaling of three major pathways: the MAP kinase pathway, PI3K-AKT-mTOR pathway, and the tumour invasion and metastasis-inducing protein 1 (TIAM1-RAC) and RAS-related protein (RAL) pathways. The MAPK pathway consists of RAS, RAF, MEK and ERK phosphorylation and regulates cell-cycle and cellular proliferation. RAS activation and dimerization leads to conformational changes that allows binding and phosphorylation of RAF molecules. In the case of mutant RAS, its dimers allow for increased RAF binding and activation. This constitutes the major downstream signaling pathway of mutant RAS. The final enzyme in the MAPK pathway, ERK translocates to the nucleus and activates various transcription factors. This promotes cellular proliferation and differentiation.

Phosphatidylinositol-4,5-bisphosphate 3-kinase (PI3K) activation by GTP-RAS complex leads to phosphorylation of phosphatidylinositol 4,5-biphosphate (PIP2) to phosphatidylinositol 3,4,5-triphosphate (PIP3). PIP3 then phosphorylates AKT, which in turn causes downstream mTOR, FOXO and NF-kB phosphorylation, leading to cell survival and resistance to apoptosis. The PI3K pathway is frequently upregulated in RAS mutations; however, the role of RAS on PI3K activation in normal cells remains unclear.

Thus, KRAS in its normal state is responsible as a key link between several cell-cycle pathways and an activating KRAS mutation leads to oncogenesis by multiple downstream activation pathways.

KRAS G12C is the commonest KRAS mutation in NSCLC, seen in about 14% of all lung adenocarcinomas, followed by G12V. The spectrum of KRAS mutations in lung cancer is heterogenous. While G12C is the commonest KRAS mutation among smokers (44%), followed by G12V (19%), G12D is the most frequent mutation in never-smokers (56%). Intriguingly, G12C was more commonly mutated in women than other KRAS mutations despite lower tobacco exposure than men, suggesting increased susceptibility to acquiring smoking-dependent G12C mutation in women. In another retrospective analysis of 2327 patients with KRAS-mutant NSCLC, KRAS G12D mutation was enriched in never-smokers (22%) or lower pack-year smoking history (median 22.5 pack years). In pancreatic cancers, KRAS G12D and G12V are the predominant mutations, at 40% and 32% of all KRAS mutations, respectively, while G12R mutation accounts for nearly 17% of all KRAS mutations in pancreatic cancer. Right-sided colorectal tumors have a predilection for KRAS mutations and G12 mutations make up about 65% of all KRAS mutations. Like in pancreatic cancers, G12D and G12V are most frequent mutations.

A detailed description of KRAS and its function can be found, e.g., in Mustachio, L. M., et al. “Targeting KRAS in cancer: promising therapeutic strategies.” Cancers 13.6 (2021): 1204; Parikh, K., et al. “Drugging KRAS: current perspectives and state-of-art review.” Journal of Hematology & Oncology 15.1 (2022): 152; and Huang, L., et al. “KRAS mutation: from undruggable to druggable in cancer.” Signal Transduction and Targeted Therapy 6.1 (2021): 386; each of which is incorporated by reference in its entirety.

The present disclosure provides “TCR-like” antibodies targeting KRAS peptide-MHC complexes. The development of these TCR-like antibodies based therapeutics can improve the therapeutic efficacy. Thus, in one aspect, the present disclosure provides methods of treating disorders associated with KRAS oncogenic mutations. In some embodiments, the disorder is cancer.

Among these peptides presented by cells, KRAS G12V (7-16) peptide (SEQ ID NO: 80) is a peptide that can be presented by HLA-A3 (e.g., HLA-A0301 (HLA-A*03:01), HLA-A0302 (HLA-A*03:02)) or HLA-A11 (e.g., HLA-A1101 (HLA-A*11:01)). In some embodiments, the antibodies or antigen binding fragments as described herein specifically bind to the KRAS/HLA-A3 complex.

### Anti-KRAS/HLA Antibodies and Antigen-Binding Fragments

The disclosure provides antibodies and antigen-binding fragments thereof that specifically bind to a MHC complex comprising a KRAS peptide. In some embodiments, the MHC molecule is HLA (e.g., any of the HLA-A3 or HLA-All described herein). The antibodies and antigen-binding fragments described herein are capable of binding to a MHC complex comprising a KRAS peptide. In some embodiments, the KRAS peptide described herein includes a hydrophobic amino acid residue (e.g., valine) at a position corresponding to Gly12 (G12) in SEQ ID NO: 108. In some embodiments, the KRAS peptide described herein includes a cysteine at a position corresponding to Gly12 (G12) in SEQ ID NO: 108. In some embodiments, the KRAS peptide described herein includes a negatively charged amino acid (e.g., aspartic acid) at a position corresponding to Gly12 (G12) in SEQ ID NO: 108. In some embodiments, the KRAS peptide described herein includes a mutation at a position corresponding to Gly12 (G12) in SEQ ID NO: 108 to valine (V), cysteine (C), or aspartic acid (D).

In some embodiments, the KRAS peptide includes a valine at a position corresponding to Gly12 of human KRAS (SEQ ID NO: 108). In some embodiments, these antibodies can increase immune response.

The disclosure provides, e.g., anti-KRAS/HLA antibodies 1A7, 1B6, 1C10, 1E12, 1F1, 1F3, 1F9, 1G1, 1G10, 1H2, 2B12, and P03141, the chimeric antibodies thereof, and the human or humanized antibodies thereof.

The CDR sequences for 1A7, and 1A7 derived antibodies (e.g., humanized antibodies) include CDRs of the heavy chain variable domain, SEQ ID NOs: 1, 2, 3, and CDRs of the light chain variable domain, SEQ ID NOs: 52, 53, 54, as defined by Kabat definition. The CDRs can also be defined by Chothia system. Under the Chothia definition, the CDR sequences of the heavy chain variable domain are set forth in SEQ ID NOs: 28, 29, 30, and CDR sequences of the light chain variable domain are set forth in SEQ ID NOs: 52, 53, 54.

The CDR sequences for 1F3, and 1F3 derived antibodies include CDRs of the heavy chain variable domain, SEQ ID NOs: 4, 5, 6, and CDRs of the light chain variable domain, SEQ ID NOs: 55, 56, 57, as defined by Kabat definition. Under Chothia definition, the CDR sequences of the heavy chain variable domain are set forth in SEQ ID NOs: 28, 29, 30, and CDRs of the light chain variable domain are set forth in SEQ ID NOs: 55, 56, 57.

The CDR sequences for 1B6, and 1B6 derived antibodies include CDRs of the heavy chain variable domain, SEQ ID NOs: 7, 8, 9, and CDRs of the light chain variable domain, SEQ ID NOs: 55, 56, 57, as defined by Kabat definition. Under Chothia definition, the CDR sequences of the heavy chain variable domain are set forth in SEQ ID NOs: 31, 32, 33, and CDRs of the light chain variable domain are set forth in SEQ ID NOs: 55, 56, 57.

The CDR sequences for 1C10, and 1C10 derived antibodies include CDRs of the heavy chain variable domain, SEQ ID NOs: 7, 8, 9, and CDRs of the light chain variable domain, SEQ ID NOs: 55, 56, 57, as defined by Kabat definition. Under Chothia definition, the CDR sequences of the heavy chain variable domain are set forth in SEQ ID NOs: 31, 32, 33, and CDRs of the light chain variable domain are set forth in SEQ ID NOs: 55, 56, 57.

The CDR sequences for 1E12, and 1E12 derived antibodies include CDRs of the heavy chain variable domain, SEQ ID NOs: 10, 11, 12, and CDRs of the light chain variable domain, SEQ ID NOs: 55, 56, 57, as defined by Kabat definition. Under Chothia definition, the CDR sequences of the heavy chain variable domain are set forth in SEQ ID NOs: 34, 35, 36, and CDRs of the light chain variable domain are set forth in SEQ ID NOs: 55, 56, 57.

The CDR sequences for 1F1, and 1F1 derived antibodies include CDRs of the heavy chain variable domain, SEQ ID NOs: 13, 14, 15, and CDRs of the light chain variable domain, SEQ ID NOs: 55, 56, 57, as defined by Kabat definition. Under Chothia definition, the CDR sequences of the heavy chain variable domain are set forth in SEQ ID NOs: 37, 38, 39, and CDRs of the light chain variable domain are set forth in SEQ ID NOs: 55, 56, 57.

The CDR sequences for 1F9, and 1F9 derived antibodies include CDRs of the heavy chain variable domain, SEQ ID NOs: 13, 14, 15, and CDRs of the light chain variable domain, SEQ ID NOs: 55, 56, 57, as defined by Kabat definition. Under Chothia definition, the CDR sequences of the heavy chain variable domain are set forth in SEQ ID NOs: 37, 38, 39, and CDRs of the light chain variable domain are set forth in SEQ ID NOs: 55, 56, 57.

The CDR sequences for 1G1, and 1G1 derived antibodies include CDRs of the heavy chain variable domain, SEQ ID NOs: 16, 17, 18, and CDRs of the light chain variable domain, SEQ ID NOs: 55, 56, 57, as defined by Kabat definition. Under Chothia definition, the CDR sequences of the heavy chain variable domain are set forth in SEQ ID NOs: 40, 41, 42, and CDRs of the light chain variable domain are set forth in SEQ ID NOs: 55, 56, 57.

The CDR sequences for 1G10, and 1G10 derived antibodies include CDRs of the heavy chain variable domain, SEQ ID NOs: 19, 20, 21, and CDRs of the light chain variable domain, SEQ ID NOs: 55, 56, 57, as defined by Kabat definition. Under Chothia definition, the CDR sequences of the heavy chain variable domain are set forth in SEQ ID NOs: 43, 44, 45, and CDRs of the light chain variable domain are set forth in SEQ ID NOs: 55, 56, 57.

The CDR sequences for 1H2, and 1H2 derived antibodies include CDRs of the heavy chain variable domain, SEQ ID NOs: 22, 23, 24, and CDRs of the light chain variable domain, SEQ ID NOs: 55, 56, 57, as defined by Kabat definition. Under Chothia definition, the CDR sequences of the heavy chain variable domain are set forth in SEQ ID NOs: 46, 47, 48, and CDRs of the light chain variable domain are set forth in SEQ ID NOs: 55, 56, 57.

The CDR sequences for 2B12, and 2B12 derived antibodies include CDRs of the heavy chain variable domain, SEQ ID NOs: 25, 26, 27, and CDRs of the light chain variable domain, SEQ ID NOs: 58, 59, 60, as defined by Kabat definition. Under Chothia definition, the CDR sequences of the heavy chain variable domain are set forth in SEQ ID NOs: 49, 50, 51, and CDRs of the light chain variable domain are set forth in SEQ ID NOs: 58, 59, 60.

The CDR sequences for P03141, and P03141 derived antibodies include CDRs of the heavy chain variable domain, SEQ ID NOs: 110, 111, 112, and CDRs of the light chain variable domain, SEQ ID NOs: 55, 56, 57, as defined by Kabat definition. Under Chothia definition, the CDR sequences of the heavy chain variable domain are set forth in SEQ ID NOs: 113, 114, 115, and CDRs of the light chain variable domain are set forth in SEQ ID NOs: 55, 56, 57.

The amino acid sequence for the heavy chain variable region of 1A7 antibody is set forth in SEQ ID NO: 61. The amino acid sequence for the light chain variable region of 1A7 antibody is set forth in SEQ ID NO: 71.

The amino acid sequence for the heavy chain variable region of 1F3 antibody is set forth in SEQ ID NO: 62. The amino acid sequence for the light chain variable region of 1F3 antibody is set forth in SEQ ID NO: 72.

The amino acid sequence for the heavy chain variable region of 1B6 antibody is set forth in SEQ ID NO: 63. The amino acid sequence for the light chain variable region of 1B6 antibody is set forth in SEQ ID NO: 73.

The amino acid sequence for the heavy chain variable region of 1C10 antibody is set forth in SEQ ID NO: 64. The amino acid sequence for the light chain variable region of 1C10 antibody is set forth in SEQ ID NO: 74.

The amino acid sequence for the heavy chain variable region of 1E12 antibody is set forth in SEQ ID NO: 65. The amino acid sequence for the light chain variable region of 1E12 antibody is set forth in SEQ ID NO: 75.

The amino acid sequence for the heavy chain variable region of 1F1 antibody is set forth in SEQ ID NO: 66. The amino acid sequence for the light chain variable region of 1F1 antibody is set forth in SEQ ID NO: 75.

The amino acid sequence for the heavy chain variable region of 1F9 antibody is set forth in SEQ ID NO: 66. The amino acid sequence for the light chain variable region of 1F9 antibody is set forth in SEQ ID NO: 72.

The amino acid sequence for the heavy chain variable region of 1G1 antibody is set forth in SEQ ID NO: 67. The amino acid sequence for the light chain variable region of 1G1 antibody is set forth in SEQ ID NO: 75.

The amino acid sequence for the heavy chain variable region of 1G10 antibody is set forth in SEQ ID NO: 68. The amino acid sequence for the light chain variable region of 1G10 antibody is set forth in SEQ ID NO: 76.

The amino acid sequence for the heavy chain variable region of 1H2 antibody is set forth in SEQ ID NO: 69. The amino acid sequence for the light chain variable region of 1H2 antibody is set forth in SEQ ID NO: 77.

The amino acid sequence for the heavy chain variable region of 2B12 antibody is set forth in SEQ ID NO: 70. The amino acid sequence for the light chain variable region of 2B12 antibody is set forth in SEQ ID NO: 78.

The amino acid sequence for the heavy chain variable region of P03141 antibody is set forth in SEQ ID NO: 116. The amino acid sequence for the light chain variable region of P03141 antibody is set forth in SEQ ID NO: 117.

The amino acid sequences for heavy chain variable regions and light variable regions of the modified antibodies are also provided. In some embodiments, the heavy chain variable region is at least 80%, 85%, 90%, 91%, 92%, 93%, 94%, 95%, 96%, 97%, 98%, or 99% identical to SEQ ID NO: 61, 62, 63, 64, 65, 66, 67, 68, 69, 70 or 116. In some embodiments, the light chain variable region is at least 80%, 85%, 90%, 91%, 92%, 93%, 94%, 95%, 96%, 97%, 98%, or 99% identical to SEQ ID NO: 71, 72, 73, 74, 75, 76, 77, 78 or 117. The heavy chain variable region sequence can be paired with the corresponding light chain variable region sequence, and together they bind to a KRAS/HLA complex.

In some embodiments, the variable regions are fully human, e.g., derived from human heavy chain immunoglobulin locus sequences (e.g., recombination of human IGHV, IGHD, and IGHJ genes), and/or human kappa chain immunoglobulin locus sequences (e.g., recombination of human IGKV and IGKJ genes).

Furthermore, in some embodiments, the antibodies or antigen-binding fragments thereof described herein can also contain one, two, or three heavy chain variable region CDRs selected from the group of SEQ ID NOs: 1-3, SEQ ID NOs: 4-6, SEQ ID NOs: 7-9, SEQ ID NOs: 10-12, SEQ ID NOs: 13-15, SEQ ID NOs: 16-18, SEQ ID NOs: 19-21, SEQ ID NOs: 22-24, SEQ ID NOs: 25-27, SEQ ID NOs: 28-30, SEQ ID NOs: 31-33, SEQ ID NOs: 34-36, SEQ ID NOs: 37-39, SEQ ID NOs: 40-42, SEQ ID NOs: 43-45, SEQ ID NOs: 46-48, SEQ ID NOs: 49-51, SEQ ID NOs: 110-112, and SEQ ID NOs: 113-115; and/or one, two, or three light chain variable region CDRs selected from the group of SEQ ID NOs: 52-54, SEQ ID NOs: 55-57, and SEQ ID NOs: 58-60.

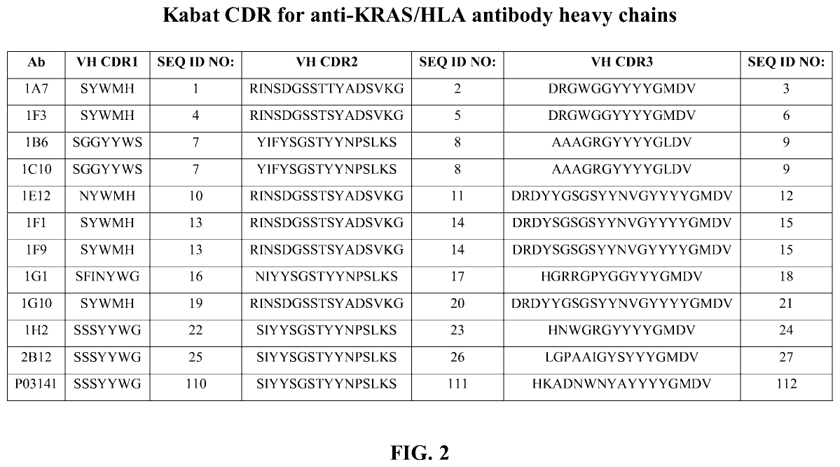

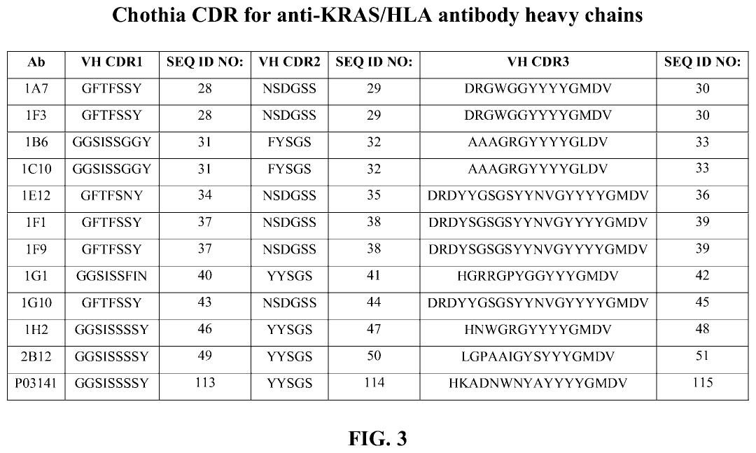

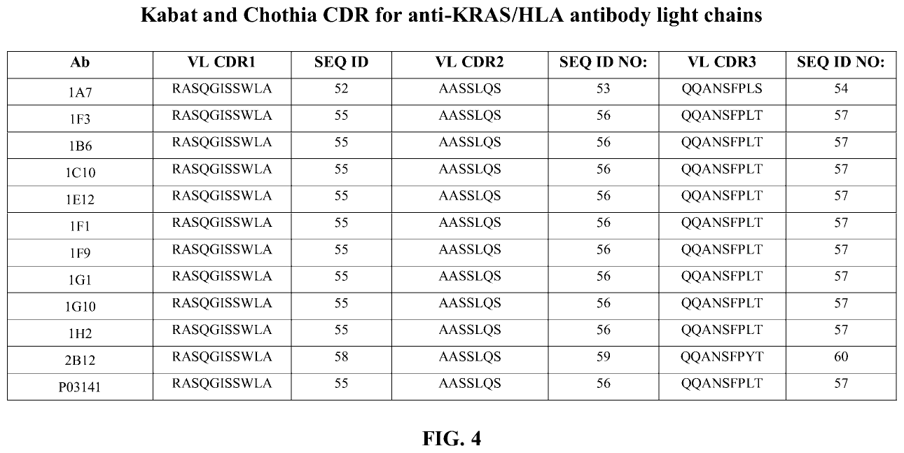

In some embodiments, the antibodies can have a heavy chain variable region (VH) comprising complementarity determining regions (CDRs) 1, 2, 3, wherein the CDR1 region comprises or consists of an amino acid sequence that is at least 80%, 85%, 90%, or 95% identical to a selected VH CDR1 amino acid sequence, the CDR2 region comprises or consists of an amino acid sequence that is at least 80%, 85%, 90%, or 95% identical to a selected VH CDR2 amino acid sequence, and the CDR3 region comprises or consists of an amino acid sequence that is at least 80%, 85%, 90%, or 95% identical to a selected VH CDR3 amino acid sequence. In some embodiments, the antibodies can have a light chain variable region (VL) comprising CDRs 1, 2, 3, wherein the CDR1 region comprises or consists of an amino acid sequence that is at least 80%, 85%, 90%, or 95% identical to a selected VL CDR1 amino acid sequence, the CDR2 region comprises or consists of an amino acid sequence that is at least 80%, 85%, 90%, or 95% identical to a selected VL CDR2 amino acid sequence, and the CDR3 region comprises or consists of an amino acid sequence that is at least 80%, 85%, 90%, or 95% identical to a selected VL CDR3 amino acid sequence. The selected VH CDRs 1, 2, 3 amino acid sequences are shown in FIG. 2 (CDRs under Kabat definition) and FIG. 3 (CDRs under Chothia definition). The selected VL CDRs 1, 2, 3 amino acid sequences are shown in FIG. 4 (CDRs under Kabat/Chothia definition).

In some embodiments, the antibody or an antigen-binding fragment described herein can contain a heavy chain variable domain containing one, two, or three of the CDRs of SEQ ID NO: 1 with zero, one or two amino acid insertions, deletions, or substitutions; SEQ ID NO: 2 with zero, one or two amino acid insertions, deletions, or substitutions; SEQ ID NO: 3 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 4 with zero, one or two amino acid insertions, deletions, or substitutions; SEQ ID NO: 5 with zero, one or two amino acid insertions, deletions, or substitutions; SEQ ID NO: 6 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 7 with zero, one or two amino acid insertions, deletions, or substitutions; SEQ ID NO: 8 with zero, one or two amino acid insertions, deletions, or substitutions; SEQ ID NO: 9 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 10 with zero, one or two amino acid insertions, deletions, or substitutions; SEQ ID NO: 11 with zero, one or two amino acid insertions, deletions, or substitutions; SEQ ID NO: 12 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 13 with zero, one or two amino acid insertions, deletions, or substitutions; SEQ ID NO: 14 with zero, one or two amino acid insertions, deletions, or substitutions; SEQ ID NO: 15 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 16 with zero, one or two amino acid insertions, deletions, or substitutions; SEQ ID NO: 17 with zero, one or two amino acid insertions, deletions, or substitutions; SEQ ID NO: 18 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 19 with zero, one or two amino acid insertions, deletions, or substitutions; SEQ ID NO: 20 with zero, one or two amino acid insertions, deletions, or substitutions; SEQ ID NO: 21 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 22 with zero, one or two amino acid insertions, deletions, or substitutions; SEQ ID NO: 23 with zero, one or two amino acid insertions, deletions, or substitutions; SEQ ID NO: 24 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 25 with zero, one or two amino acid insertions, deletions, or substitutions; SEQ ID NO: 26 with zero, one or two amino acid insertions, deletions, or substitutions; SEQ ID NO: 27 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 28 with zero, one or two amino acid insertions, deletions, or substitutions; SEQ ID NO: 29 with zero, one or two amino acid insertions, deletions, or substitutions; SEQ ID NO: 30 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 31 with zero, one or two amino acid insertions, deletions, or substitutions; SEQ ID NO: 32 with zero, one or two amino acid insertions, deletions, or substitutions; SEQ ID NO: 33 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 34 with zero, one or two amino acid insertions, deletions, or substitutions; SEQ ID NO: 35 with zero, one or two amino acid insertions, deletions, or substitutions; SEQ ID NO: 36 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 37 with zero, one or two amino acid insertions, deletions, or substitutions; SEQ ID NO: 38 with zero, one or two amino acid insertions, deletions, or substitutions; SEQ ID NO: 39 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 40 with zero, one or two amino acid insertions, deletions, or substitutions; SEQ ID NO: 41 with zero, one or two amino acid insertions, deletions, or substitutions; SEQ ID NO: 42 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 43 with zero, one or two amino acid insertions, deletions, or substitutions; SEQ ID NO: 44 with zero, one or two amino acid insertions, deletions, or substitutions; SEQ ID NO: 45 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 46 with zero, one or two amino acid insertions, deletions, or substitutions; SEQ ID NO: 47 with zero, one or two amino acid insertions, deletions, or substitutions; SEQ ID NO: 48 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 49 with zero, one or two amino acid insertions, deletions, or substitutions; SEQ ID NO: 50 with zero, one or two amino acid insertions, deletions, or substitutions; SEQ ID NO: 51 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 110 with zero, one or two amino acid insertions, deletions, or substitutions; SEQ ID NO: 111 with zero, one or two amino acid insertions, deletions, or substitutions; SEQ ID NO: 112 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 113 with zero, one or two amino acid insertions, deletions, or substitutions; SEQ ID NO: 114 with zero, one or two amino acid insertions, deletions, or substitutions; SEQ ID NO: 115 with zero, one or two amino acid insertions, deletions, or substitutions.

In some embodiments, the antibody or an antigen-binding fragment described herein can contain a light chain variable domain containing one, two, or three of the CDRs of SEQ ID NO: 52 with zero, one or two amino acid insertions, deletions, or substitutions; SEQ ID NO: 53 with zero, one or two amino acid insertions, deletions, or substitutions; SEQ ID NO: 54 with zero, one or two amino acid insertions, deletions, or substitutions.

In some embodiments, the antibody or an antigen-binding fragment described herein can contain a light chain variable domain containing one, two, or three of the CDRs of SEQ ID NO: 55 with zero, one or two amino acid insertions, deletions, or substitutions; SEQ ID NO: 56 with zero, one or two amino acid insertions, deletions, or substitutions; SEQ ID NO: 57 with zero, one or two amino acid insertions, deletions, or substitutions.

In some embodiments, the antibody or an antigen-binding fragment described herein can contain a light chain variable domain containing one, two, or three of the CDRs of SEQ ID NO: 58 with zero, one or two amino acid insertions, deletions, or substitutions; SEQ ID NO: 59 with zero, one or two amino acid insertions, deletions, or substitutions; SEQ ID NO: 60 with zero, one or two amino acid insertions, deletions, or substitutions.

The insertions, deletions, and substitutions can be within the CDR sequence, or at one or both terminal ends of the CDR sequence. In some embodiments, the CDR is determined based on Kabat definition scheme. In some embodiments, the CDR is determined based on Chothia definition scheme. In some embodiments, the CDR is determined based on a combination of Kabat and Chothia definition scheme. In some embodiments, the CDR is determined based on IMGT definition. In some embodiments, the CDR is determined based on contact definition.

The disclosure also provides antibodies or antigen-binding fragments thereof that bind to a KRAS/HLA complex. The antibodies or antigen-binding fragments thereof contain a heavy chain variable region (VH) comprising or consisting of an amino acid sequence that is at least 80%, 85%, 90%, or 95% identical to a selected VH sequence, and a light chain variable region (VL) comprising or consisting of an amino acid sequence that is at least 80%, 85%, 90%, or 95% identical to a selected VL sequence. In some embodiments, the selected VH sequence is SEQ ID NO: 61, and the selected VL sequence is SEQ ID NO: 71. In some embodiments, the selected VH sequence is SEQ ID NO: 62 and the selected VL sequence is SEQ ID NO: 72. In some embodiments, the selected VH sequence is SEQ ID NO: 63 and the selected VL sequence is SEQ ID NO: 73. In some embodiments, the selected VH sequence is SEQ ID NO: 64 and the selected VL sequence is SEQ ID NO: 74. In some embodiments, the selected VH sequence is SEQ ID NO: 65 and the selected VL sequence is SEQ ID NO: 75. In some embodiments, the selected VH sequence is SEQ ID NO: 66 and the selected VL sequence is SEQ ID NO: 75. In some embodiments, the selected VH sequence is SEQ ID NO: 66 and the selected VL sequence is SEQ ID NO: 72. In some embodiments, the selected VH sequence is SEQ ID NO: 67 and the selected VL sequence is SEQ ID NO: 75. In some embodiments, the selected VH sequence is SEQ ID NO: 68 and the selected VL sequence is SEQ ID NO: 76. In some embodiments, the selected VH sequence is SEQ ID NO: 69 and the selected VL sequence is SEQ ID NO: 77. In some embodiments, the selected VH sequence is SEQ ID NO: 70 and the selected VL sequence is SEQ ID NO: 78. In some embodiments, the selected VH sequence is SEQ ID NO: 116 and the selected VL sequence is SEQ ID NO: 117.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90%, 95%, or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For example, the comparison of sequences and determination of percent identity between two sequences can be accomplished using a Blossum 62 scoring matrix with a gap penalty of 12, a gap extend penalty of 4, and a frameshift gap penalty of 5.

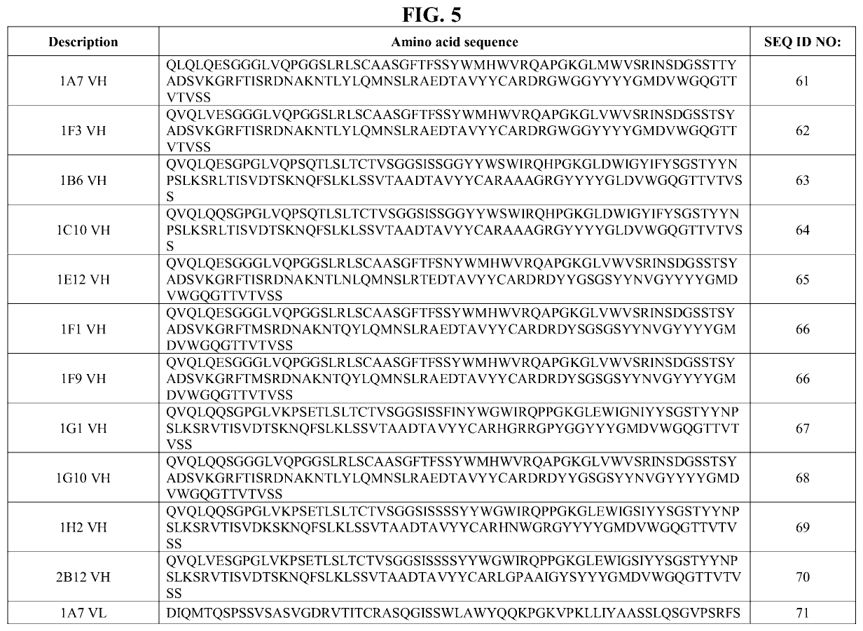

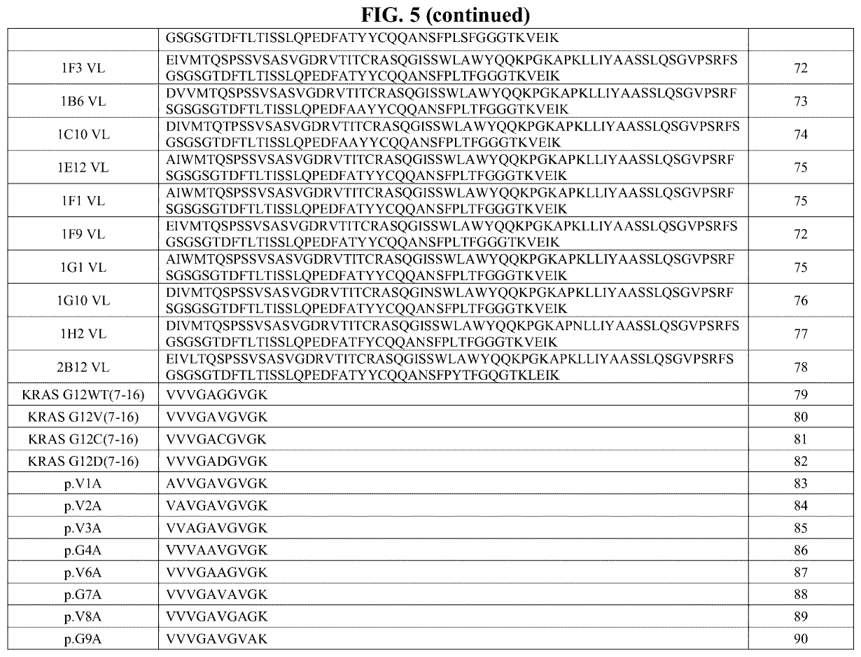

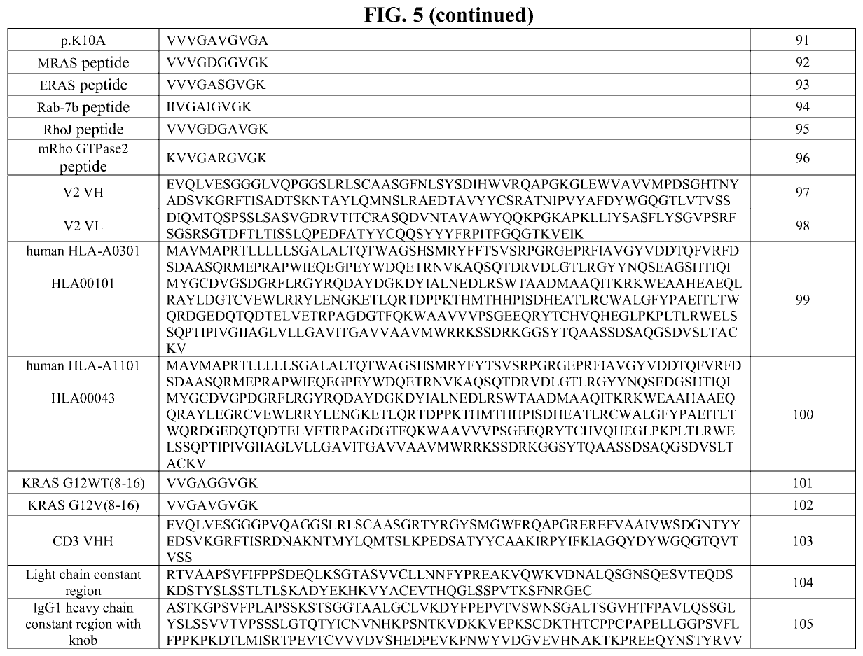

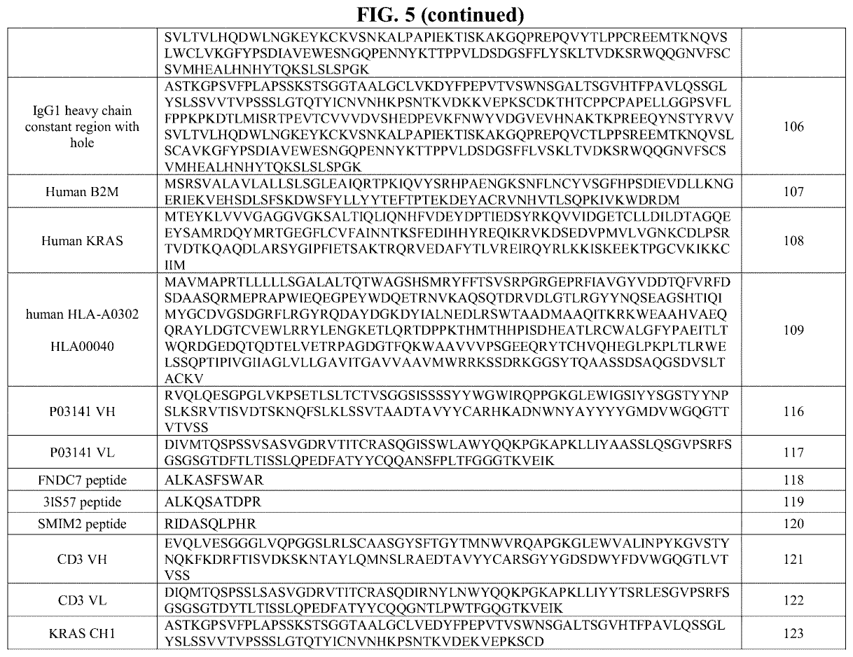

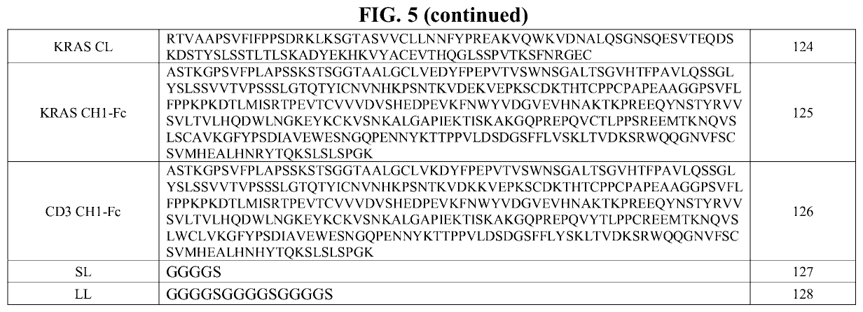

The disclosure also provides nucleic acid comprising a polynucleotide encoding a polypeptide comprising an immunoglobulin heavy chain or an immunoglobulin light chain. The immunoglobulin heavy chain or immunoglobulin light chain comprises CDRs as shown in FIG. 2, FIG. 3, or FIG. 4, or have sequences as shown in FIG. 5. When the polypeptides are paired with corresponding polypeptide (e.g., a corresponding heavy chain variable region or a corresponding light chain variable region), the paired polypeptides bind to a KRAS/HLA complex.

The anti-KRAS/HLA antibodies and antigen-binding fragments can also be antibody variants (including derivatives and conjugates) of antibodies or antibody fragments and multi-specific (e.g., bi-specific) antibodies or antibody fragments. Additional antibodies provided herein are polyclonal, monoclonal, multimeric, multi-specific (e.g., bi-specific), human antibodies, chimeric antibodies (e.g., human-mouse chimera), single-chain antibodies, intracellularly-made antibodies (i.e., intrabodies), and antigen-binding fragments thereof. The antibodies or antigen-binding fragments thereof can be of any type (e.g., IgG, IgE, IgM, IgD, IgA, and IgY), class (e.g., IgG1, IgG2, IgG3, IgG4, IgA1, and IgA2), or subclass. In some embodiments, the antibody or antigen-binding fragment thereof is an IgG antibody or antigen-binding fragment thereof.

Fragments of antibodies are suitable for use in the methods provided so long as they retain the desired affinity and specificity of the full-length antibody. Thus, a fragment of an antibody that binds to a KRAS/HLA complex will retain an ability to bind to the KRAS/HLA complex. An Fv fragment is an antibody fragment which contains a complete antigen recognition and binding site. This region consists of a dimer of one heavy and one light chain variable domain in tight association, which can be covalent in nature, for example in scFv. It is in this configuration that the three CDRs of each variable domain interact to define an antigen binding site on the surface of the VH-VL dimer. Collectively, the six CDRs or a subset thereof confer antigen binding specificity to the antibody. However, even a single variable domain (or half of an Fv comprising only three CDRs specific for an antigen) can have the ability to recognize and bind antigen, although usually at a lower affinity than the entire binding site. Single-chain Fv or (scFv) antibody fragments comprise the VH and VL domains (or regions) of antibody, wherein these domains are present in a single polypeptide chain. Generally, the scFv polypeptide further comprises a polypeptide linker between the VH and VL domains, which enables the scFv to form the desired structure for antigen binding.

The present disclosure also provides an antibody or antigen-binding fragment thereof that cross-competes with any antibody or antigen-binding fragment as described herein. The cross-competing assay is known in the art, and is described e.g., in Moore et al., “Antibody cross-competition analysis of the human immunodeficiency virus type 1 gp120 exterior envelope glycoprotein.” Journal of Virology 70.3 (1996): 1863-1872, which is incorporated herein reference in its entirety. In one aspect, the present disclosure also provides an antibody or antigen-binding fragment thereof that binds to the same epitope or region as any antibody or antigen-binding fragment as described herein. The epitope binning assay is known in the art, and is described e.g., in Estep et al. “High throughput solution-based measurement of antibody-antigen affinity and epitope binning.” MAbs. Vol. 5. No. 2. Taylor & Francis, 2013, which is incorporated herein reference in its entirety.

### Antibodies and Antigen Binding Fragments

The present disclosure provides various antibodies and antigen-binding fragments thereof derived from anti-KRAS/HLA antibodies described herein. In general, antibodies (also called immunoglobulins) are made up of two classes of polypeptide chains, light chains and heavy chains. A non-limiting examples of antibody of the present disclosure can be an intact, four immunoglobulin chain antibody comprising two heavy chains and two light chains. The heavy chain of the antibody can be of any isotype including IgM, IgG, IgE, IgA, or IgD or sub-isotype including IgG1, IgG2, IgG2a, IgG2b, IgG3, IgG4, IgE1, IgE2, etc. The light chain can be a kappa light chain or a lambda light chain. An antibody can comprise two identical copies of a light chain and two identical copies of a heavy chain. The heavy chains, which each contain one variable domain (or variable region, VH) and multiple constant domains (or constant regions), bind to one another via disulfide bonding within their constant domains to form the “stem” of the antibody. The light chains, which each contain one variable domain (or variable region, VL) and one constant domain (or constant region), each bind to one heavy chain via disulfide binding. The variable region of each light chain is aligned with the variable region of the heavy chain to which it is bound. The variable regions of both the light chains and heavy chains contain three hypervariable regions sandwiched between more conserved framework regions (FR).

These hypervariable regions, known as the complementary determining regions (CDRs), form loops that comprise the antigen binding surface of the antibody. The four framework regions largely adopt a beta-sheet conformation and the CDRs form loops connecting the beta-sheet structure, and in some cases forming part of, the beta-sheet structure. The CDRs in each chain are held in close proximity by the framework regions and, with the CDRs from the other chain, contribute to the formation of the antigen-binding region.

Methods for identifying the CDR regions of an antibody by analyzing the amino acid sequence of the antibody are well known, and a number of definitions of the CDRs are commonly used. The Kabat definition is based on sequence variability, and the Chothia definition is based on the location of the structural loop regions. These methods and definitions are described in, e.g., Martin, “Protein sequence and structure analysis of antibody variable domains,” Antibody engineering, Springer Berlin Heidelberg, 2001. 422-439; Abhinandan, et al. “Analysis and improvements to Kabat and structurally correct numbering of antibody variable domains,” Molecular immunology 45.14 (2008): 3832-3839; Wu, T. T. and Kabat, E. A. (1970) J. Exp. Med. 132:211-250; Martin et al., Methods Enzymol. 203:121-53 (1991); Morea et al., Biophys Chem. 68 (1-3): 9-16 (October 1997); Morea et al., J Mol Biol. 275 (2): 269-94 (January 1998); Chothia et al., Nature 342 (6252): 877-83 (December 1989); Ponomarenko and Bourne, BMC Structural Biology 7:64 (2007); each of which is incorporated herein by reference in its entirety.

The CDRs are important for recognizing an epitope of an antigen. As used herein, an “epitope” is the smallest portion of a target molecule capable of being specifically bound by the antigen binding domain of an antibody. The minimal size of an epitope may be about three, four, five, six, or seven amino acids, but these amino acids need not be in a consecutive linear sequence of the antigen's primary structure, as the epitope may depend on an antigen's three-dimensional configuration based on the antigen's secondary and tertiary structure.

In some embodiments, the antibody is an intact immunoglobulin molecule (e.g., IgG1, IgG2a, IgG2b, IgG3, IgM, IgD, IgE, IgA). The IgG subclasses (IgG1, IgG2, IgG3, and IgG4) are highly conserved, differ in their constant region, particularly in their hinges and upper CH2 domains. The sequences and differences of the IgG subclasses are known in the art, and are described, e.g., in Vidarsson, et al, “IgG subclasses and allotypes: from structure to effector functions.” Frontiers in immunology 5 (2014); Irani, et al. “Molecular properties of human IgG subclasses and their implications for designing therapeutic monoclonal antibodies against infectious diseases.” Molecular immunology 67.2 (2015): 171-182; Shakib, Farouk, ed. The human IgG subclasses: molecular analysis of structure, function and regulation. Elsevier, 2016; each of which is incorporated herein by reference in its entirety.

The antibody can also be an immunoglobulin molecule that is derived from any species (e.g., human, rodent, mouse, camelid). Antibodies disclosed herein also include, but are not limited to, polyclonal, monoclonal, monospecific, polyspecific antibodies, and chimeric antibodies that include an immunoglobulin binding domain fused to another polypeptide. The term “antigen binding domain” or “antigen binding fragment” is a portion of an antibody that retains specific binding activity of the intact antibody, i.e., any portion of an antibody that is capable of specific binding to an epitope on the intact antibody's target molecule. It includes, e.g., Fab, Fab′, F(ab′)₂, and variants of these fragments. Thus, in some embodiments, an antibody or an antigen binding fragment thereof can be, e.g., a scFv, a Fv, a Fd, a dAb, a bispecific antibody, a bispecific scFv, a diabody, a linear antibody, a single-chain antibody molecule, a multi-specific antibody formed from antibody fragments, and any polypeptide that includes a binding domain which is, or is homologous to, an antibody binding domain. Non-limiting examples of antigen binding domains include, e.g., the heavy chain and/or light chain CDRs of an intact antibody, the heavy and/or light chain variable regions of an intact antibody, full length heavy or light chains of an intact antibody, or an individual CDR from either the heavy chain or the light chain of an intact antibody.

Fragments of antibodies are suitable for use in the methods described herein are also provided. The Fab fragment contains a variable and constant domain of the light chain and a variable domain and the first constant domain (CH1) of the heavy chain. F(ab′)₂ antibody fragments comprise a pair of Fab fragments which are generally covalently linked near their carboxy termini by hinge cysteines between them. Other chemical couplings of antibody fragments are also known in the art.

Diabodies are small antibody fragments with two antigen-binding sites, which fragments comprise a VH connected to a VL in the same polypeptide chain (VH and VL). By using a linker that is too short to allow pairing between the two domains on the same chain, the domains are forced to pair with the complementary domains of another chain and create two antigen-binding sites.

Linear antibodies comprise a pair of tandem Fd segments (VH-CH1-VH-CH1) which, together with complementary light chain polypeptides, form a pair of antigen binding regions. Linear antibodies can be bispecific or monospecific.

Antibodies and antibody fragments of the present disclosure can be modified in the Fc region to provide desired effector functions or serum half-life.

Multimerization of antibodies may be accomplished through natural aggregation of antibodies or through chemical or recombinant linking techniques known in the art. For example, some percentage of purified antibody preparations (e.g., purified lgG| molecules) spontaneously form protein aggregates containing antibody homodimers and other higher-order antibody multimers.

Alternatively, antibody homodimers may be formed through chemical linkage techniques known in the art. For example, heterobifunctional crosslinking agents including, but not limited to SMCC (succinimidyl 4-(maleimidomethyl)cyclohexane-1-carboxylate) and SATA (N-succinimidyl S-acethylthio-acetate) can be used to form antibody multimers. An exemplary protocol for the formation of antibody homodimers is described in Ghetie et al. (Proc. Natl. Acad. Sci. U.S.A. 94:7509-7514, 1997). Antibody homodimers can be converted to Fab′2 homodimers through digestion with pepsin. Another way to form antibody homodimers is through the use of the autophilic T15 peptide described in Zhao et al. (J. Immunol. 25:396-404, 2002).

In some embodiments, the multi-specific antibody is a bi-specific antibody. Bi-specific antibodies can be made by engineering the interface between a pair of antibody molecules to maximize the percentage of heterodimers that are recovered from recombinant cell culture. For example, the interface can contain at least a part of the CH3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers. This method is described, e.g., in WO 96/27011, which is incorporated by reference in its entirety.

Bi-specific antibodies include cross-linked or “heteroconjugate” antibodies. For example, one of the antibodies in the heteroconjugate can be coupled to avidin and the other to biotin. Heteroconjugate antibodies can also be made using any convenient cross-linking methods. Suitable cross-linking agents and cross-linking techniques are well known in the art and are disclosed in U.S. Pat. No. 4,676,980, which is incorporated herein by reference in its entirety.

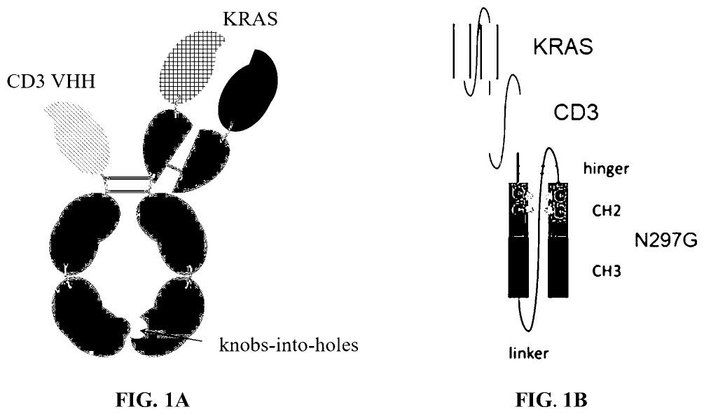

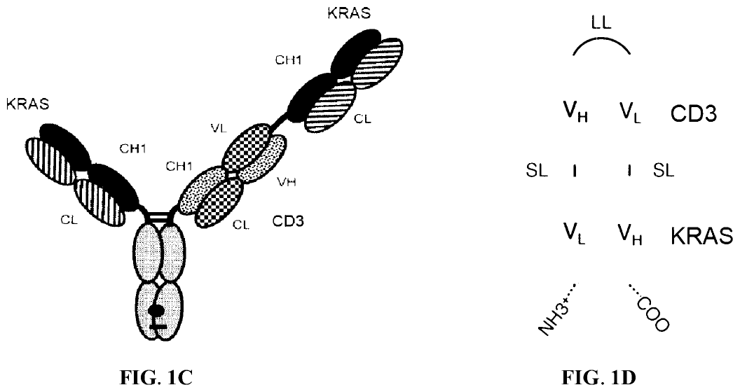

In some embodiments, the disclosure is related to a bispecific antibody that comprises (1) a first functional moiety comprising an antibody or antigen-binding fragment described herein; (2) a second functional moiety comprising a T-cell engaging molecule; and (3) a third functional moiety comprising a single chain human crystallizable fragment. In some embodiments, the T-cell engaging molecule targets CD3. In some embodiments, the T-cell engaging molecule is an anti-CD3 VHH. In some embodiments, the antibody or antigen-binding fragment described herein is linked to a human crystallizable fragment via a hinge region, and an anti-CD3 antibody or antigen-binding fragment thereof is linked to the same human crystallizable fragment via a hinge region. FIG. 1A illustrates an exemplary structure of a bispecific antibody.

In some embodiments, the bispecific antibody is a anti-KRAS/CD3 antibody, e.g. a BiTe, a (scFv) 2, a nanobody, a nanobody-HSA, a DART, a TandAb, a scDiabody, a scDiabody-CH3, scFv-CH-CL-scFv, a HSAbody, scDiabody-HAS, ScDb or a tandem-scFv. In some embodiments, the anti-KRAS/CD3 antibody is a VHH-scAb, a VHH-Fab, a Dual scFab, a F(ab′)₂, a diabody, a crossMab, a DAF (two-in-one), a DAF (four-in-one), a DutaMab, a DT-IgG, a knobs-in-holes common light chain, a knobs-in-holes assembly, a charge pair, a Fab-arm exchange, a SEEDbody, a LUZ-Y, a Fcab, a KA-body, an orthogonal Fab, a DVD-IgG, a IgG(H)-scFv, a scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig, Zybody, DVI-IgG, Diabody-CH3, a triple body, a miniantibody, a minibody, a TriBi minibody, scFv-CH3 KIH, Fab-scFv, a F(ab′)₂-scFv2, a scFv-KIH, a Fab-scFv-Fc, a tetravalent HCAb, a scDiabody-Fc, a Diabody-Fc, a tandem scFv-Fc, an Intrabody, a dock and lock, a ImmTAC, an lgG-IgG conjugate, a Cov-X-Body, or a scFv1-PEG-scFv2.

Any of the antibodies or antigen-binding fragments described herein may be conjugated to a stabilizing molecule (e.g., a molecule that increases the half-life of the antibody or antigen-binding fragment thereof in a subject or in solution). Non-limiting examples of stabilizing molecules include: a polymer (e.g., a polyethylene glycol) or a protein (e.g., serum albumin, such as human serum albumin). The conjugation of a stabilizing molecule can increase the half-life or extend the biological activity of an antibody or an antigen-binding fragment in vitro (e.g., in tissue culture or when stored as a pharmaceutical composition) or in vivo (e.g., in a human).

In some embodiments, the antibodies or antigen-binding fragments described herein can be conjugated to a therapeutic agent. The antibody-drug conjugate comprising the antibody or antigen-binding fragment thereof can covalently or non-covalently bind to a therapeutic agent. In some embodiments, the therapeutic agent is a cytotoxic or cytostatic agent (e.g., cytochalasin B, gramicidin D, ethidium bromide, emetine, mitomycin, etoposide, teniposide, vincristine, vinblastine, colchicin, doxorubicin, daunorubicin, dihydroxyanthracin, maytansinoids such as DM-1 and DM-4, dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs).

In some embodiments, the KRAS peptide comprises a sequence that is at least 80%, 85%, 90%, 95%, or 100% identical to the amino acid sequence of SEQ ID NO: 80.

In some embodiment, the antibodies or antigen-binding fragments described herein does not bind to a MHC molecule without the KRAS peptide.

In some embodiments, the antibodies or antigen-binding fragments thereof described herein can specifically bind to a KRAS/HLA-A3 complex. In some embodiments, the complex comprises a KRAS peptide. In some embodiments, the KRAS peptide includes a valine at a position corresponding to Gly12 of human KRAS (SEQ ID NO: 108). Because KRAS mutations are present in many cancer cells, the antibodies or antigen-binding fragments thereof described herein with a high binding affinity and specificity to the KRAS can be used to form part of a chimeric antigen receptor (CAR). In some embodiments, the chimeric antigen receptors are fusions of single-chain variable fragments (scFv) as described herein, fused to CD3-zeta transmembrane- and endodomain. In some embodiments, the chimeric antigen receptor also comprises intracellular signaling domains from various costimulatory protein receptors (e.g., CD28, 41BB, ICOS). In some embodiments, the chimeric antigen receptor comprises multiple signaling domains, e.g., CD3z-CD28-41BB or CD3z-CD28-OX40, to increase potency. Thus, in one aspect, the disclosure further provides cells (e.g., T cells) that express the chimeric antigen receptors as described herein.

In some embodiments, the scFv has one heavy chain variable domain, and one light chain variable domain. In some embodiments, the scFv has two heavy chain variable domains, and two light chain variable domains.

In some embodiments, sequences (e.g., CDRs or VH/VL sequences) of the antibody or antigen-binding fragment thereof described herein can be used to generate a multi-specific antibody (e.g., a bispecific antibody) targeting a KRAS/HLA complex and an additional antigen (e.g., OX40, CD3, 4-1BB, CD314, CD47, PD-1, CTLA4, CD40 or PDL1). In some embodiments, the additional antigen is a T cell-specific antigen (e.g., CD3, CD4, or CD8). For example, the multi-specific or bispecific antibodies described herein can recruit and activate T cells for killing cancer cells expressing KRAS G12V.

In some embodiments, provided herein are multi-specific antibodies (e.g., bispecific antibodies) having an anti-KRAS arm and an anti-CD3 arm. In some embodiments, the anti-KRAS arm includes a heavy chain (e.g., any of the heavy chains having a VH described herein), and a light chain (e.g., any of the light chains having a VL described herein). In some embodiments, the anti-CD3 arm includes a heavy chain variable region (VHH) of an anti-CD3 heavy chain antibody. In some embodiments, the VHH comprises or consists of an amino acid sequence that is at least 80%, 85%, 86%, 87%, 88%, 89%, 90%, 91%, 92%, 93%, 94%, 95%, 96%, 97%, 98%, 99% or 100% identical to SEQ ID NO: 103. In some embodiments, the bispecific antibodies described herein have a schematic structure shown in FIG. 1A.

In some embodiments, the bispecific antibody described herein is an anti-KRAS/CD3 antibody. In some embodiments, the bispecific antibody in present disclosure is designed to be 1+1 (monovalent for each target) and has an IgG1 subtype structure. In some embodiments, the bispecific antibody can bridge target cells (e.g., cells expressing KRAS G12V) and effector cells (e.g., T cells expressing CD3), thereby allowing the effector cells to kill target cells. In some embodiments, the anti-KRAS/CD3 antibodies described herein can specifically induce effector cells (e.g., CD3+ T cells) to kill target cells (e.g., cells expressing KRAS G12V) at an E:T ratio of about 1:1, about 2:1, about 3:1, about 4:1, about 5:1, about 6:1, about 7:1, about 8:1, about 9:1, or about 10:1. In some embodiments, the anti-KRAS/CD3 antibodies described herein do not induce effector cells (e.g., CD3+ T cells) to kill cells not expressing KRAS G12V (e.g., cells expressing wild-type KRAS).

In some embodiments, the anti-KRAS/CD3 antibodies or antigen-binding fragments thereof have a light chain constant region that is at least 80%, 85%, 86%, 87%, 88%, 89%, 90%, 91%, 92%, 93%, 94%, 95%, 96%, 97%, 98%, 99% or 100% identical to SEQ ID NO: 104, and a heavy chain constant region that is at least 80%, 85%, 86%, 87%, 88%, 89%, 90%, 91%, 92%, 93%, 94%, 95%, 96%, 97%, 98%, 99% or 100% identical to any one of SEQ ID NOs: 105 and 106.

In some embodiments, the anti-KRAS/CD3 antibodies include knobs-into-holes (KIH) mutations. In some embodiments, the anti-KRAS/CD3 antibody includes a first antigen-binding domain that specifically binds to KRAS, and a second antigen-binding domain that specifically binds to CD3. In some embodiments, the first antigen-binding domain includes a heavy chain that including one or more knob mutations (a knob heavy chain), and the second antigen-binding domain includes a heavy chain including one or more hole mutations (a hole heavy chain). In some embodiments, the first antigen-binding domain includes a heavy chain that includes one or more hole mutations (a hole heavy chain), and the second antigen-binding domain that includes a heavy chain including one or more knob mutations (a knob heavy chain). In some embodiments, the anti-KRAS/CD3 antibody includes a knob heavy chain comprising a constant region that is at least 80%, 85%, 86%, 87%, 88%, 89%, 90%, 91%, 92%, 93%, 94%, 95%, 96%, 97%, 98%, 99% or 100% identical to SEQ ID NO: 105. In some embodiments, the anti-KRAS/CD3 antibody includes a hole heavy chain comprising a constant region that is at least 80%, 85%, 86%, 87%, 88%, 89%, 90%, 91%, 92%, 93%, 94%, 95%, 96%, 97%, 98%, 99% or 100% identical to SEQ ID NO: 106.

### Antibody Drug Conjugates (ADC)

The antibodies, the antigen-binding fragments thereof, or the antigen-binding protein constructs (e.g., bispecific antibodies) described herein can be conjugated to a therapeutic agent (a drug). The therapeutic agent can be covalently or non-covalently bind to the antibody or antigen-binding fragment or the antigen binding protein construct (e.g., a bispecific antibody). In some embodiments, the bispecific antibody has a common light chain.

In some embodiments, the therapeutic agent is a cytotoxic or cytostatic agent (e.g., monomethyl auristatin E, monomethyl auristatin F, cytochalasin B, gramicidin D, ethidium bromide, emetine, mitomycin, etoposide, tenoposide, vincristine, vinblastine, colchicin, doxorubicin, daunorubicin, dihydroxy anthracin, maytansinoids such as DM-1 and DM-4, dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs). Useful classes of cytotoxic, cytostatic, or immunomodulatory agents include, for example, antitubulin agents, DNA minor groove binders, DNA replication inhibitors, and alkylating agents.

In some embodiments, the therapeutic agent can include, but not limited to, cytotoxic reagents, such as chemo-therapeutic agents, immunotherapeutic agents and the like, antiviral agents or antimicrobial agents. In some embodiments, the therapeutic agent to be conjugated can be selected from, but not limited to, MMAE (monomethyl auristatin E), MMAD (monomethyl auristatin D), or MMAF (monomethyl auristatin F).

In some embodiments, the therapeutic agent is an auristatin, such as auristatin E (also known in the art as a derivative of dolastatin-10) or a derivative thereof. The auristatin can be, for example, an ester formed between auristatin E and a keto acid. For example, auristatin E can be reacted with paraacetyl benzoic acid or benzoylvaleric acid to produce AEB and AEVB, respectively. Other typical auristatins include AFP, MMAF, and MMAE. The synthesis and structure of exemplary auristatins are described in U.S. Patent Application Publication No. 2003-0083263; International Patent Publication No. WO 04/010957, International Patent Publication No. WO 02/088172, and U.S. Pat. Nos. 7,498,298, 6,884,869, 6,323,315; 6,239,104; 6,034,065; 5,780,588; 5,665,860; 5,663,149; 5,635,483; 5,599,902; 5,554,725; 5,530,097; 5,521,284; 5,504,191; 5,410,024; 5,138,036; 5,076,973; 4,986,988; 4,978,744; 4,879,278; 4,816,444; and 4,486,414, each of which is incorporated by reference herein in its entirety and for all purposes.

Auristatins have been shown to interfere with microtubule dynamics and nuclear and cellular division and have anticancer activity. Auristatins bind tubulin and can exert a cytotoxic or cytostatic effect on cancer cell. There are a number of different assays, known in the art, which can be used for determining whether an auristatin or resultant antibody-drug conjugate exerts a cytostatic or cytotoxic effect on a desired cell.

In some embodiments, the therapeutic agent is a chemotherapeutic agent. Examples of chemotherapeutic agents include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK7; razoxane; sizofiran; spirogermanium; tenuazonic acid; triaziquone; 2′,2′,2′-trichlorotriethylamine; urethan; vindesine; dacarbazine; mannomustine; mitobronitol; mitolactol; pipobroman; gacytosine; arabinoside (“Ara-C”); cyclophosphamide; taxanes, e.g. paclitaxel (TAXOL®, Bristol-Myers Squibb Oncology, Princeton, N.J.) and doxetaxel (TAXOTERE®, Rhone-Poulenc Rorer, Antony, France); chlorambucil; gemcitabine; 6-thioguanine; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4 (5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above. A detailed description of the chemotherapeutic agents can be found in, e.g., US20180193477A1, which is incorporated by reference in its entirety.

In some embodiments, the antigen-binding construct is coupled to the drug via a cleavable linker e.g. a SPBD linker or a maleimidocaproyl-valine-citrulline-p-aminobenzyloxycarbonyl (VC) linker. In some embodiments, the antigen-binding construct is coupled to the drug via a non-cleavable linker e.g. a MCC linker formed using SMCC or sulfo-SMCC. Selection of an appropriate linker for a given ADC can be readily made by the skilled person having knowledge of the art and taking into account relevant factors, such as the site of attachment to the antigen binding construct, any structural constraints of the drug and the hydrophobicity of the drug. A number of specific linker-toxin combinations have been described and may be used with the antigen binding constructs described herein to prepare ADCs in certain embodiments. Examples include, but are not limited to, cleavable peptide-based linkers with auristatins such as MMAE and MMAF, camptothecins such as SN-38, duocarmycins and PBD dimers; non-cleavable MC-based linkers with auristatins MMAF and MMAE; acid-labile hydrazone-based linkers with calicheamicins and doxorubicin; disulfide-based linkers with maytansinoids such as DM1 and DM4, and bis-maleimido-trioxyethylene glycol (BMPEO)-based linkers with maytansinoid DM1. Some these therapeutic agents and linkers are described, e.g., in Peters & Brown, (2015) Biosci. Rep. e00225; Dosio et al., (2014) Recent Patents on Anti-Cancer Drug Discovery 9:35-65; US Patent Publication No. US 2015/0374847, and US20180193477A1; which are incorporated herein by reference in the entirety.

Depending on the desired drug and selected linker, those skilled in the art can select suitable method for coupling them together. For example, some conventional coupling methods, such as amine coupling methods, can be used to form the desired drug-linker complex which still contains reactive groups for conjugating to the antibodies through covalent linkage. In some embodiments, a drug-maleimide complex (i.e., maleimide linking drug) can be used for the payload bearing reactive group in the present disclosure. Most common reactive group capable of bonding to thiol group in ADC preparation is maleimide. Additionally, organic bromides, iodides also are frequently used.

The ADC can be prepared by one of several routes known in the art, employing organic chemistry reactions, conditions, and reagents known to those skilled in the art (see, for example, Bioconjugate Techniques (G. T. Hermanson, 2013, Academic Press). For example, conjugation can be achieved by (1) reaction of a nucleophilic group or an electrophilic group of an antibody with a bivalent linker reagent, to form antibody-linker intermediate Ab-L, via a covalent bond, followed by reaction with an activated drug moiety D; or (2) reaction of a nucleophilic group or an electrophilic group of a drug moiety with a linker reagent, to form drug-linker intermediate D-L, via a covalent bond, followed by reaction with the nucleophilic group or an electrophilic group of an antibody. Conjugation methods (1) and (2) can be employed with a variety of antibodies, drug moieties, and linkers to prepare the ADCs described here. Various prepared linkers, linker components and toxins are commercially available or may be prepared using standard synthetic organic chemistry techniques. These methods are described e.g., in March's Advanced Organic Chemistry (Smith & March 2006, Sixth Ed., Wiley); Toki et al., (2002) J. Org. Chem. 67:1866-1872; Frisch et al., (1997) Bioconj. Chem. 7:180-186; Bioconjugate Techniques (G. T. Hermanson, 2013, Academic Press); US20210379193A1, and US20180193477A1, which are incorporated herein by reference in the entirety. In addition, a number of pre-formed drug-linkers suitable for reaction with a selected antigen binding construct are also available commercially, for example, linker-toxins comprising DM1, DM4, MMAE, MMAF or Duocarmycin SA are available from Creative BioLabs (Shirley, N.Y.).

Several specific examples of methods of preparing ADCs are known in the art and are described in U.S. Pat. No. 8,624,003 (pot method), U.S. Pat. No. 8,163,888 (one-step), and U.S. Pat. No. 5,208,020 (two-step method), and US20180193477A1, which are incorporated herein by reference in the entirety. Other methods are known in the art and include those described in Antibody-Drug Conjugates: Methods in Molecular Biology, 2013, Ducry (Ed.), Springer.

Drug loading is represented by the number of drug moieties per antibody in a molecule of ADC. For some antibody-drug conjugates, the drug loading may be limited by the number of attachment sites on the antibody. For example, where the attachment is a cysteine thiol, as in certain exemplary embodiments described herein, the drug loading may range from 0 to 8 drug moieties per antibody. In certain embodiments, higher drug loading, e.g. p≥5, may cause aggregation, insolubility, toxicity, or loss of cellular permeability of certain antibody-drug conjugates. In certain embodiments, the average drug loading for an antibody-drug conjugate ranges from 1 to about 8; from about 2 to about 6; or from about 3 to about 5. Indeed, it has been shown that for certain antibody-drug conjugates, the optimal ratio of drug moieties per antibody can be around 4. In some embodiments, the drug-to-antibody ratio (DAR) is about or at least 1, 2, 3, 4, 5, 6, 7, or 8. In some embodiments, the average DAR in the composition is about 1~about 2, about 2~about 3, about 3~about 4, about 3~about 5, about 4~about 5, about 5~about 6, about 6~about 7, or about 7~about 8.

### Antibody and ADC Characteristics

In some implementations, the antibody (or antigen-binding fragments thereof) specifically binds to a KRAS/HLA complex with a dissociation rate (koff) of less than 0.1 s⁻¹, less than 0.01 s⁻¹, less than 0.001 s⁻¹, less than 0.0001 s⁻¹, or less than 0.00001 s⁻¹. In some embodiments, the dissociation rate (koff) is greater than 0.01 s⁻¹, greater than 0.001 s⁻¹, greater than 0.0001 s⁻¹, greater than 0.00001 s⁻¹, or greater than 0.000001 s⁻¹.

In some embodiments, kinetic association rates (kon) is greater than 1×10²/Ms, greater than 1×10³/Ms, greater than 1×10⁴/Ms, greater than 1×10⁵/Ms, or greater than 1×10⁶/Ms. In some embodiments, kinetic association rates (kon) is less than 1×10⁵/Ms, less than 1×10⁶/Ms, or less than 1×10⁷/Ms.

Affinities can be deduced from the quotient of the kinetic rate constants (KD=koff/kon). In some embodiments, KD is less than 1×10⁻⁶ M, less than 1×10⁻⁷M, less than 1×10⁻⁸ M, less than 1×10⁻⁹ M, or less than 1×10⁻¹⁰ M. In some embodiments, the KD is less than 50 nM, 30 nM, 20 nM, 15 nM, 10 nM, 9 nM, 8 nM, 7 nM, 6 nM, 5 nM, 4 nM, 3 nM, 2 nM, or 1 nM. In some embodiments, KD is greater than 1×10⁻⁷M, greater than 1×10⁻⁸ M, greater than 1×10⁻⁹ M, greater than 1×10⁻¹⁰ M, greater than 1×10⁻¹¹ M, or greater than 1×10⁻¹² M.

In some embodiments, the key targeting sites of the anti-KRAS/HLA antibodies or antigen binding fragments thereof on KRAS G12V (7-16) peptide (SEQ ID NO: 80) are different from those of the antibody V2 scFv analog.

In some embodiments, the anti-KRAS/HLA antibody binds to positions 3, 4, 5, 6, 7, and/or 8 of the KRAS peptide in the KRAS G12V (7-16) peptide/HLA-A3 complex. In some embodiments, the anti-KRAS/HLA antibody binds to positions 5, 6, and/or 7 of the KRAS peptide in the KRAS G12V (7-16) peptide/HLA complex. In some embodiments, the anti-KRAS/HLA antibody specially recognize position 6 of the KRAS peptide in the KRAS G12V (7-16) peptide/HLA complex.

In some embodiments, the binding affinity, dissociation rate, and association rate are determined between the antibodies or the antigen-binding fragments thereof, and a complex including a KRAS peptide (e.g., any of the KRAS peptide described herein), a HLA (e.g., HLA-A*03:01), and optionally with B2M.

General techniques for measuring the affinity of an antibody for an antigen include, e.g., ELISA, RIA, and surface plasmon resonance (SPR). In some embodiments, the antibodies and antigen-binding fragments described herein are capable of binding to a human KRAS G12V peptide (e.g., KRAS G12V (7-16) peptide (SEQ ID NO: 80). In some embodiments, the antibodies and antigen-binding fragments described herein are not capable of binding to a human KRAS G12V peptide (e.g., KRAS G12V (8-16) (SEQ ID NO: 102)). In some embodiments, these antibodies are not capable of binding to a wild-type human KRAS peptide (e.g., KRAS G12WT (7-16) peptide (SEQ ID NO: 79), or KRAS G12WT (8-16) peptide (SEQ ID NO: 101)). In some embodiments, these antibodies are not capable of binding to a human KRAS peptide that includes a mutation at Gly12 to a non-valine residue (e.g., KRAS G12C (7-16) peptide (SEQ ID NO: 81), or KRAS G12D (7-16) peptide (SEQ ID NO: 82)). In some embodiments, the antibodies and antigen-binding fragments described herein can bind to a human KRAS protein having a G12V mutation (a human KRAS G12V protein). In some embodiments, the antibodies and antigen-binding fragments described herein does not bind to peptides derived from MRAS protein, ERAS protein, Rab-7b protein, RhoJ protein, or mRho GTPase2 protein, e.g., MRAS peptide (SEQ ID NO: 92), ERAS peptide (SEQ ID NO: 93), Rab-7b peptide (SEQ ID NO: 94), RhoJ peptide (SEQ ID NO: 95), mRho GTPase2 peptide (SEQ ID NO: 96), FNDC7 peptide (SEQ ID NO: 118), 3IS57 peptide (SEQ ID NO: 119), or SMIM2 peptide (SEQ ID NO: 120).

In some embodiments, the antibody or antigen-binding fragment thereof described herein can specifically bind to a human KRAS G12V protein, but not a human KRAS protein including a mutation at Gly12 to a non-valine residue, e.g., cysteine or aspartic acid; or a wild-type KRAS protein. In some embodiments, the antibody or antigen-binding fragment thereof described herein has a binding affinity to a human KRAS G12V protein or a fragment (e.g., a peptide) thereof that is at least 1-fold, 2-fold, 3-fold, 4-fold, 5-fold, 6-fold, 7-fold, 8-fold, 9-fold, 10-fold, 20-fold, 50-fold, 100-fold, 200-fold, 500-fold, or 1000-fold higher than that to a wild-type human KRAS protein, a human KRAS protein having a non-valine residue at the Gly12 position, or a fragment (e.g., a peptide) thereof.

In some embodiments, the antibody or antigen-binding fragment thereof described herein can specifically bind to cells pulsed with a human KRAS protein or a peptide thereof (e.g., any of the KRAS peptide described herein). For example, the cells can be incubated with 10 μM-1 mM (e.g., 50 μM) human KRAS peptide (e.g., any of the KRAS peptide described herein) for at least 10 minutes, 20 minutes, 30 minutes, 40 minutes, 50 minutes, 1 hour, 2 hours, 3 hours, 4 hours, 5 hours, 6 hours, 7 hours, 8 hours, 9 hours, 10 hours, 11 hours, or 12 hours. In some embodiments, the cell also expresses a HLA. Because the anti-KRAS/HLA antibodies described herein are generated by immunizing MHC-I VH/VL mice with a fusion protein including HLA-A*03:01, in some embodiments, the antibody or antigen-binding fragment thereof described herein can specifically bind to a human HLA-A3 (e.g., HLA-A*03:01 or HLA-A*03:02). In some embodiments, the antibody or antigen-binding fragment thereof described herein does not bind to cells pulsed with a human KRAS protein or a peptide thereof, wherein the cells do not express a human HLA-A3, or express a HLA-All (e.g., HLA-A*11:01).

In some embodiments, the binding activities described herein are determined between the antibodies or the antigen-binding fragments thereof, and cells expressing a HLA (e.g., HLA-A*03:01) that are pulsed with a KRAS G12V peptide (e.g., any of the KRAS G12V peptide described herein). In some embodiments, the KRAS G12V peptide is KRAS G12V (7-16) peptide (SEQ ID NO: 80).

In some embodiments, the antibody or antigen-binding fragment thereof described herein has a tumor growth inhibition percentage (TGI_{TV}%) that is greater than 10%, 20%, 30%, 40%, 50%, 60%, 70%, 80%, 90%, 100%, 110%, 120%, 130%, 140%, 150%, 160%, 170%, 180%, 190%, or 200%. In some embodiments, the antibody has a tumor growth inhibition percentage that is less than 60%, 70%, 80%, 90%, 100%, 110%, 120%, 130%, 140%, 150%, 160%, 170%, 180%, 190%, or 200%. The TGI % can be determined, e.g., at 3, 4, 5, 6, 7, 8, 9, 10, 11, 12, 13, 14, 15, 16, 17, 18, 19, 20, 21, 22, 23, 24, 25, 26, 27, 28, 29, or 30 days after the treatment starts, or 1, 2, 3, 4, 5, 6, 7, 8, 9, 10, 11, or 12 months after the treatment starts. As used herein, the tumor growth inhibition percentage (TGI %) is calculated using the following formula:

TGI (%)=[1−(Tᵢ−T₀)/(Vᵢ−V₀)]×100%

Tᵢ is the average tumor volume in the treatment group on Day i. To is the average tumor volume in the treatment group on Day zero. Vᵢ is the average tumor volume in the control group on Day i. V₀ is the average tumor volume in the control group on Day zero.

In some embodiments, the antibodies or antigen-binding fragments thereof as described herein are antagonists of a KRAS G12V protein. In some embodiments, the antibodies or antigen-binding fragments thereof as described herein are agonists of a KRAS G12V protein.

In some embodiments, the antibodies or antigen-binding fragments thereof as described herein are not toxic. In some embodiments, no significant differences in body weight can be observed between treatment group and control group.

In some embodiments, the antibodies or antigen binding fragments can induce complement-dependent cytotoxicity (CDC) and/or antibody dependent cellular cytotoxicity (ADCC), and kill the tumor cell.

In some embodiments, the antibodies or antigen binding fragments thereof have a functional Fc region. In some embodiments, effector function of a functional Fc region is antibody-dependent cell-mediated cytotoxicity (ADCC). In some embodiments, effector function of a functional Fc region is phagocytosis. In some embodiments, effector function of a functional Fc region is ADCC and phagocytosis. In some embodiments, the antibodies or antigen binding fragments thereof can induce complement-dependent cytotoxicity (CDC).

In some embodiments, the Fc region is human IgG1, human IgG2, human IgG3, or human IgG4. In some embodiments, the antibody is a human IgG1 antibody, optionally with SI mutations, LALA mutations, N297A mutation, YTE mutations, and/or FLAA mutations. In some embodiments, the antibody is a human IgG4 antibody, optionally with SI mutations, LALA mutations, N297A mutation, YTE mutations, and/or FLAA mutations.

In some embodiments, the antibodies or antigen binding fragments do not have a functional Fc region. For example, the antibodies or antigen binding fragments are Fab, Fab′, F(ab′)₂, and Fv fragments. In some embodiments, the Fc region has LALA mutations (L234A and L235A mutations in EU numbering), or LALA-PG mutations (L234A, L235A, P329G mutations in EU numbering). In some embodiments, the Fc region has FLAA mutations (F234A and L235A according to EU numbering). In some embodiments, the Fc has SI mutations (S239D and 1332E mutations according to EU numbering). In some embodiments, the Fc has N297A mutation according to EU numbering. In some embodiments, the Fc has YTE mutations (M252Y, S254T and T256E according to EU numbering).

In some embodiments, the Fc have a SI mutation (S239D and 1332E mutations in EU numbering).

### Methods of Making Anti-KRAS/HLA Antibodies

A synthesized and purified KRAS/MHC complex can be used as an immunogen to generate antibodies using standard techniques for polyclonal and monoclonal antibody preparation. In some embodiments, MHC-I VH/VL mice (Detailed descriptions of MHC-I VH/VL mice can be found e.g., in PCT/CN2022/081924) can be immunized with a KRAS peptide (e.g., KRAS G12V (7-16) peptide, SEQ ID NO: 80) presented specifically by human HLA-A03.

Polyclonal antibodies can be raised in animals by multiple injections (e.g., subcutaneous or intraperitoneal injections) of an antigenic peptide or protein. In some embodiments, the antigenic peptide or protein is injected with at least one adjuvant. In some embodiments, the antigenic peptide or protein can be conjugated to an agent that is immunogenic in the species to be immunized. Animals can be injected with the antigenic peptide or protein more than one time (e.g., twice, three times, or four times).

An immunogen typically is used to prepare antibodies by immunizing a suitable subject (e.g., human or transgenic animal expressing at least one human immunoglobulin locus). An appropriate immunogenic preparation can contain, for example, a recombinantly-expressed or a chemically-synthesized polypeptide. The preparation can further include an adjuvant, such as Freund's complete or incomplete adjuvant, or a similar immunostimulatory agent.

In some embodiments, the animals used for immunization are MHC-I VH/VL mice. Details of the MHC-I VH/VL mice can be found, e.g., in PCT/CN2022/081924, which is incorporated herein by reference in the entirety. The MHC-I VH/VL mice can generate fully human or humanized antibodies upon immunization, and also express a humanized MHC protein complex. Thus, an antigen peptide-MHC complex can be used for immunizing the mice to generate a diverse collection of antibodies. In some embodiments, the components of the fusion protein can form a complex, e.g., KRAS G12V/HLA-A0301 complex, that can be recognized by the humanized MHC protein complex expressed in the MHC-I VH/VL mice. As a result, the immunized MHC-I VH/VL mice can produce immunoglobulin light chain variable domains (e.g., any of the VLs described herein) that can pair with a rather diverse family of heavy chain variable domains (e.g., any of the VHs described herein), including e.g., affinity matured or somatically mutated variable domains.

The full-length polypeptide or protein can be used or, alternatively, antigenic peptide fragments thereof can be used as immunogens. The antigenic peptide of a protein comprises at least 8 (e.g., at least 5, 6, 7, 8, 9, 10, 11, 12, 13, 14, 15, 16, 17, 18, 19, 20, 25, or 30) amino acid residues of the amino acid sequence of a KRAS protein (e.g., a human KRAS G12V protein) and encompasses an epitope of the protein such that an antibody raised against the peptide forms a specific immune complex with the protein. For instance, KRAS G12V (7-16) peptide (SEQ ID NO: 80) can be used as immunogens to generate antibodies against the human KRAS G12V protein.

An immunogen typically is used to prepare antibodies by immunizing a suitable subject (e.g., human or transgenic animal expressing at least one human immunoglobulin locus). An appropriate immunogenic preparation can contain, for example, a recombinantly-expressed or a chemically-synthesized polypeptide (e.g., a fragment of human KRAS). The preparation can further include an adjuvant, such as Freund's complete or incomplete adjuvant, or a similar immunostimulatory agent.

olyclonal antibodies can be prepared as described above by immunizing a suitable subject with a KRAS G12V protein, or an antigenic peptide thereof (e.g., part of the KRAS G12V protein, e.g., KRAS G12V (7-16) peptide (SEQ ID NO: 80)) as an immunogen. The antibody titer in the immunized subject can be monitored over time by standard techniques, such as with an enzyme-linked immunosorbent assay (ELISA) using the immobilized KRAS protein or a peptide thereof. If desired, the antibody molecules can be isolated from the mammal (e.g., from the blood) and further purified by well-known techniques, such as protein A or protein G chromatography to obtain the IgG fraction. At an appropriate time after immunization, e.g., when the specific antibody titers are highest, antibody-producing cells can be obtained from the subject and used to prepare monoclonal antibodies by standard techniques, such as the hybridoma technique originally described by Kohler et al. (Nature 256:495-497, 1975), the human B cell hybridoma technique (Kozbor et al., Immunol. Today 4:72, 1983), the EBV-hybridoma technique (Cole et al., Monoclonal Antibodies and Cancer Therapy, Alan R. Liss, Inc., pp. 77-96, 1985), or trioma techniques. The technology for producing hybridomas is well known (see, generally, Current Protocols in Immunology, 1994, Coligan et al. (Eds.), John Wiley & Sons, Inc., New York, NY). Hybridoma cells producing a monoclonal antibody are detected by screening the hybridoma culture supernatants for antibodies that bind the polypeptide or epitope of interest, e.g., using a standard ELISA assay.

Variants of the antibodies or antigen-binding fragments described herein can be prepared by introducing appropriate nucleotide changes into the DNA encoding a human, humanized, or chimeric antibody, or antigen-binding fragment thereof described herein, or by peptide synthesis. Such variants include, for example, deletions, insertions, or substitutions of residues within the amino acids sequences that make-up the antigen-binding site of the antibody or an antigen-binding domain. In a population of such variants, some antibodies or antigen-binding fragments will have increased affinity for the target protein, e.g., a KRAS/HLA complex. Any combination of deletions, insertions, and/or combinations can be made to arrive at an antibody or antigen-binding fragment thereof that has increased binding affinity for the target. The amino acid changes introduced into the antibody or antigen-binding fragment can also alter or introduce new post-translational modifications into the antibody or antigen-binding fragment, such as changing (e.g., increasing or decreasing) the number of glycosylation sites, changing the type of glycosylation site (e.g., changing the amino acid sequence such that a different sugar is attached by enzymes present in a cell), or introducing new glycosylation sites.

Antibodies disclosed herein can be derived from any species of animal, including mammals. Non-limiting examples of native antibodies include antibodies derived from humans, primates, e.g., monkeys and apes, cows, pigs, horses, sheep, camelids (e.g., camels and llamas), chicken, goats, and rodents (e.g., rats, mice, hamsters and rabbits), including transgenic rodents genetically engineered to produce human antibodies.

Human and humanized antibodies include antibodies having variable and constant regions derived from (or having the same amino acid sequence as those derived from) human germline immunoglobulin sequences. Human antibodies may include amino acid residues not encoded by human germline immunoglobulin sequences (e.g., mutations introduced by random or site-specific mutagenesis in vitro or by somatic mutation in vivo), for example in the CDRs.

A humanized antibody, typically has a human framework (FR) grafted with non-human CDRs. Thus, a humanized antibody has one or more amino acid sequence introduced into it from a source which is non-human. These non-human amino acid residues are often referred to as “import” residues, which are typically taken from an “import” variable domain. Humanization can be essentially performed by e.g., substituting rodent CDRs or CDR sequences for the corresponding sequences of a human antibody. These methods are described in e.g., Jones et al., Nature, 321:522-525 (1986); Riechmann et al., Nature, 332:323-327 (1988); Verhoeyen et al., Science, 239:1534-1536 (1988); each of which is incorporated by reference herein in its entirety. Accordingly, “humanized” antibodies are chimeric antibodies wherein substantially less than an intact human V domain has been substituted by the corresponding sequence from a non-human species. In practice, humanized antibodies are typically mouse antibodies in which some CDR residues and some FR residues are substituted by residues from analogous sites in human antibodies.

It is further important that antibodies be humanized with retention of high specificity and affinity for the antigen and other favorable biological properties. To achieve this goal, humanized antibodies can be prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved.

Ordinarily, amino acid sequence variants of the human, humanized, or chimeric anti-KRAS/HLA antibody will contain an amino acid sequence having at least 75%, 80%, 85%, 90%, 95%, 96%, 97%, 98%, or 99% percent identity with a sequence present in the light or heavy chain of the original antibody.

In some embodiments, a mouse (e.g., RenMab™ mouse) with a humanized heavy chain immunoglobulin locus and a humanized kappa chain immunoglobulin locus is used to generate antibodies. The heavy chain immunoglobulin locus is a region on the chromosome that contains genes for the heavy chains of antibodies. The locus can include e.g., human IGHV (variable) genes, human IGHD (diversity) genes, human IGHJ (joining) genes, and mouse heavy chain constant domain genes. The kappa chain immunoglobulin locus is a region on the chromosome that contains genes that encode the light chains of antibodies (kappa chain). The kappa chain immunoglobulin locus can include e.g., human IGKV (variable) genes, human IGKJ (joining) genes, and mouse light chain constant domain genes. A detailed description regarding RenMab™ mice can be found in PCT/CN2020/075698 or US20200390073A1, which is incorporated herein by reference in its entirety.

In some embodiments, a mouse (e.g., RenLite™ mouse) with a humanized heavy chain immunoglobulin locus and a humanized kappa chain immunoglobulin locus is used to generate antibodies. The heavy chain immunoglobulin locus is a region on the chromosome that contains genes for the heavy chains of antibodies. The locus can include e.g., human IGHV (variable) genes, human IGHD (diversity) genes, human IGHJ (joining) genes, and mouse heavy chain constant domain genes. The kappa chain immunoglobulin locus is a region on the chromosome that contains genes that encode a common light chains. The kappa chain immunoglobulin locus can include e.g., a human IGKV (variable) gene, a human IGKJ (joining) gene, and mouse light chain constant domain genes. A detailed description regarding RenLite™ mice can be found in PCT/CN2021/097652, which is incorporated herein by reference in its entirety.

The antibodies generated by the mice have a full human VH, a full human VL, and mouse constant regions. In some embodiments, the human VH and human VL is linked to a human IgG constant regions (e.g., IgG1, IgG2, IgG3, and IgG4).

Identity or homology with respect to an original sequence is usually the percentage of amino acid residues present within the candidate sequence that are identical with a sequence present within the human, humanized, or chimeric anti-KRAS/HLA antibody or fragment, after aligning the sequences and introducing gaps, if necessary, to achieve the maximum percent sequence identity, and not considering any conservative substitutions as part of the sequence identity.

Additional modifications to the anti-KRAS/HLA antibodies or antigen-binding fragments can be made. For example, a cysteine residue(s) can be introduced into the Fc region, thereby allowing interchain disulfide bond formation in this region. The homodimeric antibody thus generated may have any increased half-life in vitro and/or in vivo. Homodimeric antibodies with increased half-life in vitro and/or in vivo can also be prepared using heterobifunctional cross-linkers as described, for example, in Wolff et al. (Cancer Res. 53:2560-2565, 1993). Alternatively, an antibody can be engineered which has dual Fc regions (see, for example, Stevenson et al., Anti-Cancer Drug Design 3:219-230, 1989).

In some embodiments, a covalent modification can be made to the anti-KRAS/HLA antibody or antigen-binding fragment thereof. These covalent modifications can be made by chemical or enzymatic synthesis, or by enzymatic or chemical cleavage. Other types of covalent modifications of the antibody or antibody fragment are introduced into the molecule by reacting targeted amino acid residues of the antibody or fragment with an organic derivatization agent that is capable of reacting with selected side chains or the N- or C-terminal residues.

In some embodiments, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues; or position 314 in Kabat numbering); however, Asn297 may also be located about +3 amino acids upstream or downstream of position 297, i.e., between positions 294 and 300, due to minor sequence variations in antibodies. Such fucosylation variants may have improved ADCC function. In some embodiments, to reduce glycan heterogeneity, the Fc region of the antibody can be further engineered to replace the Asparagine at position 297 with Alanine (N297A).

In some embodiments, to facilitate production efficiency by avoiding Fab-arm exchange, the Fc region of the antibodies was further engineered to replace the serine at position 228 (EU numbering) of IgG4 with proline (S228P). A detailed description regarding S228 mutation is described, e.g., in Silva et al. “The S228P mutation prevents in vivo and in vitro IgG4 Fab-arm exchange as demonstrated using a combination of novel quantitative immunoassays and physiological matrix preparation.” Journal of Biological Chemistry 290.9 (2015): 5462-5469, which is incorporated by reference in its entirety.

### Chimeric Antigen Receptors (CAR)

Chimeric antigen receptors (CARs) combine many facets of normal T cell activation into a single protein. They link an extracellular antigen recognition domain to an intracellular signaling domain, which activates the T cell when an antigen is bound. CARs are typically composed of four regions: an antigen binding domain, an extracellular hinge region, a transmembrane domain, and an intracellular T cell signaling domain.

The antigen binding domain is exposed to the outside of the cell, in the ectodomain portion of the receptor. It interacts with potential target molecules and is responsible for targeting the CAR-T cell to any cell expressing a matching molecule. The antigen binding domain is typically derived from the variable regions of a monoclonal antibody linked together as a single-chain variable fragment (scFv). An scFv is a chimeric protein made up of the light (VL) and heavy (VH) chains of immunoglobulins, connected with a short linker peptide. The linker between the two chains consists of hydrophilic residues with stretches of glycine and serine in it for flexibility as well as stretches of glutamate and lysine for added solubility. In some embodiments, the antigen binding domain specifically binds to a tumor associated antigen. In some embodiments, the antigen binding domain specifically binds to a KRAS/MHC complex (e.g., a KRAS/HLA-A3 complex) as described here. In some embodiments, the antigen binding domain does not bind to the MHC molecule. In some embodiments, the antigen binding domain is derived from any antibody as described herein.

The hinge, also called a spacer, is a small structural domain that sits between the antigen binding domain and the cell's outer membrane. An ideal hinge enhances the flexibility of the scFv receptor head, reducing the spatial constraints between the CAR and its target antigen. This promotes antigen binding and synapse formation between the CAR-T cells and target cells. Hinge sequences are often based on membrane-proximal regions from immune molecules including e.g., IgG, CD8, and CD28.

The transmembrane domain is a structural component, consisting of a hydrophobic alpha helix that spans the cell membrane. It anchors the CAR to the plasma membrane, bridging the extracellular hinge and antigen binding domains with the intracellular signaling region. This domain is essential for the stability of the receptor as a whole. Generally, the transmembrane domain from the most membrane-proximal component of the endodomain is used, but different transmembrane domains result in different receptor stability. The CD28 transmembrane domain is known to result in a highly expressed, stable receptor.

The intracellular T cell signaling domain lies in the receptor's endodomain, inside the cell. After an antigen is bound to the external antigen binding domain, CAR receptors cluster together and transmit an activation signal. Then the internal cytoplasmic end of the receptor perpetuates signaling inside the T cell. Normal T cell activation relies on the phosphorylation of immunoreceptor tyrosine-based activation motifs (ITAMs) present in the cytoplasmic domain of CD3-zeta. To mimic this process, CD3-zeta's cytoplasmic domain is commonly used as the main CAR endodomain component. T cells also require co-stimulatory molecules in addition to CD3 signaling in order to persist after activation. For this reason, the endodomains of CAR receptors typically also include one or more chimeric domains from co-stimulatory proteins. Signaling domains from a wide variety of co-stimulatory molecules have been successfully tested, including CD28, CD27, CD134 (OX40), and CD137 (4-1BB).

Various CAR molecules and vectors expressing these CAR molecules can be used in the methods described herein. In some embodiments, the CAR molecules specifically binds to a tumor-associated antigen, e.g., a KRAS/HLA-A3 complex.

Exemplary structure of antigen receptors, including the hinge, the transmembrane domain, and the intracellular T cell signaling domain, and methods for engineering and introducing such receptors into cells, are described, for example, in Chandran et al., “T cell receptor-based cancer immunotherapy: Emerging efficacy and pathways of resistance.” Immunological reviews 290.1 (2019): 127-147; Cartellieri, Marc, et al., “Chimeric antigen receptor-engineered T cells for immunotherapy of cancer.” BioMed Research International 2010 (2010); and PCT publication No. WO2017173256A1; US2002/131960, US2013/287748, US2013/0149337, U.S. Pat. Nos. 6,451,995, 7,446,190, 8,252,592; each of which is incorporated herein by reference in its entirety.

The disclosure provides chimeric antigen receptors (CARs) or fragments thereof that specifically bind to a KRAS/HLA-A3 complex. The CARs or fragments thereof described herein are capable of binding to a KRAS/HLA-A3 complex.

The disclosure provides CARs or fragments thereof, comprising (a) an extracellular antigen-binding domain that specifically recognizes a KRAS/HLA-A3 complex; (b) a transmembrane domain; and (c) an intracellular signaling region. In some embodiments, the antigen-binding domain includes a heavy chain variable domain (VH) and a light chain variable domain (VL). In some embodiments, the VH and VL of the CAR or fragments thereof described herein are identical to the VH and the VL of any of the antibodies or antigen binding fragments described herein.

In some embodiments, single-chain variable fragments (scFv) of anti-KRAS/HLA antibodies (1A7, 1B6, 1C10, 1E12, 1F1, 1F3, 1F9, 1G1, 1G10, 1H2 and 2B12) can be used to generate KRAS-targeted CARs.

In some embodiments, the CAR has an anti-KRAS/HLA antigen-binding domain (e.g., an scFv derived from anti-KRAS/HLA antibody 1A7, 1B6, 1C10, 1E12, 1F1, 1F3, 1F9, 1G1, 1G10, 1H2 and/or 2B12). In some embodiments, the CAR has the below elements linked in tandem: (1) a CD8a signal peptide, (2) an anti-KRAS/HLA antigen-binding domain (e.g., an scFv derived from anti-KRAS/HLA antibody 1A7, 1B6, 1C10, 1E12, 1F1, 1F3, 1F9, 1G1, 1G10, 1H2 and/or 2B12), (3) a CD8a hinge region, (4) a CD8a transmembrane domain, (5) a 4-1BB intracellular domain, and (6) a CD35 intracellular domain.

### T Cell Receptors (TCRs)

In some embodiments, the engineered receptor described herein is a recombinant T cell receptors (TCRs). A “T cell receptor” or “TCR” refers to a molecule that contains a variable α and β chains (also known as TCRα and TCRβ, respectively) or a variable γ and δ chains (also known as TCRγ and TCRδ, respectively) and that is capable of specifically binding to an antigen peptide bound to a MHC receptor. In some embodiments, the TCR is in the αβ form. Typically, TCRs that exist in αβ and γδ forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules. In some embodiments, a TCR also can contain a constant domain, a transmembrane domain and/or a short cytoplasmic tail. For example, in some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction. Unless otherwise stated, the term “TCR” should be understood to encompass functional TCR fragments thereof. The term also encompasses intact or full-length TCRs, including TCRs in the αβ form or γδ form.

Thus, for purposes herein, reference to a TCR includes any TCR or functional fragment, such as an antigen-binding portion of a TCR that binds to a specific antigenic peptide bound in an MHC molecule, i.e., MHC-peptide complex (e.g., a KRAS/HLA-A3 complex). The antigen-binding fragment as described herein can used in a TCR. In some cases, the antigen-binding portion contains the variable domains of a TCR, such as variable α chain and variable β chain of a TCR, sufficient to form a binding site for binding to a specific MHC-peptide complex, such as generally where each chain contains three complementarity determining regions. In some embodiments, the variable α chain and variable β chain of a TCR are derived from the VH and VL as described in the present disclosure. In some embodiments, the TCR chains contain a constant domain. For example, like immunoglobulins, the extracellular portion of TCR chains (e.g., α-chain, β-chain) can contain two immunoglobulin domains, a variable domain (e.g., Va or VB; typically amino acids 1 to 116 based on Kabat numbering; Kabat et al., “Sequences of Proteins of Immunological Interest, US Dept. Health and Human Services, Public Health Service National Institutes of Health, 1991, 5^{th} ed.) at the N-terminus, and one constant domain (e.g., α-chain constant domain or Ca, typically amino acids 117 to 259 based on Kabat, β-chain constant domain or CB, typically amino acids 117 to 295 based on Kabat) adjacent to the cell membrane. For example, in some cases, the extracellular portion of the TCR formed by the two chains contains two membrane-proximal constant domains, and two membrane-distal variable domains containing CDRs. The constant domain of the TCR domain contains short connecting sequences in which a cysteine residue forms a disulfide bond, making a link between the two chains. In some embodiments, a TCR may have an additional cysteine residue in each of the α and β chains such that the TCR contains two disulfide bonds in the constant domains.

In some embodiments, the TCR chains can contain a transmembrane domain. In some embodiments, the transmembrane domain is positively charged. In some cases, the TCR chains contains a cytoplasmic tail. In some cases, the structure allows the TCR to associate with other molecules like CD3. For example, a TCR containing constant domains with a transmembrane region can anchor the protein in the cell membrane and associate with invariant subunits of the CD3 signaling apparatus or complex.

Generally, CD3 is a multi-protein complex that can possess three distinct chains (γ, δ, and ε) in mammals and the ζ-chain. For example, in mammals the complex can contain a CD3γ chain, a CD3δ chain, two CD3ε chains, and a homodimer of CD3ζ chains. The CD3γ, CD3δ, and CD3ε chains are highly related cell surface proteins of the immunoglobulin superfamily containing a single immunoglobulin domain. The transmembrane regions of the CD3γ, CD3δ, and CD3ε chains are negatively charged, which is a characteristic that allows these chains to associate with the positively charged T cell receptor chains. The intracellular tails of the CD3γ, CD3δ, and CD3ε chains each contain a single conserved motif known as an immunoreceptor tyrosine-based activation motif or ITAM, whereas each CD3ζ chain has three. Generally, ITAMs are involved in the signaling capacity of the TCR complex. These accessory molecules have negatively charged transmembrane regions and play a role in propagating the signal from the TCR into the cell. The CD3- and ζ-chains, together with the TCR, form what is known as the T cell receptor complex.

In some embodiments, the TCR may be a heterodimer of two chains α and β (or optionally γ and δ) or it may be a single chain TCR construct. In some embodiments, the TCR is a heterodimer containing two separate chains (α and β chains or γ and δ chains) that are linked, such as by a disulfide bond or disulfide bonds.

In some embodiments, sequences of the VH and/or VL of the anti-KRAS/HLA antibodies described herein (1A7, 1B6, 1C10, 1E12, 1F1, 1F3, 1F9, 1G1, 1G10, 1H2 and 2B12) can be used to generate KRAS-targeted chimeric TCRs. In some embodiments, the chimeric TCR has an anti-KRAS/HLA antigen-binding variable domain (e.g., Va or VB derived from anti-KRAS/HLA antibody 1A7, 1B6, 1C10, 1E12, 1F1, 1F3, 1F9, 1G1, 1G10, 1H2 and/or 2B12). In some embodiments, the antigen-binding fragment as described herein replaces the variable α chain and variable β chain of a TCR. In some embodiments, the antigen-binding fragment as described herein are attached to the α chain, or the β chain. In some embodiments, an scFv as described herein is attached to a TCR.

### Engineered Cells

The present disclosure provides engineered cells (e.g., immune cells, T cells, NK cells, tumor-infiltrating lymphocytes) that express CAR, and/or various proteins as described herein. These engineered cells can be used to treat various disorders or disease as described herein (e.g., KRAS-associated cancer).

In various embodiments, the cell that is engineered can be obtained from e.g., humans and non-human animals. In various embodiments, the cell that is engineered can be obtained from bacteria, fungi, humans, rats, mice, rabbits, monkeys, pig or any other species. Preferably, the cell is from humans, rats or mice. In some embodiments, the cells are mouse lymphocytes and engineered (e.g., transduced) to express the CAR, or antigen-binding fragment thereof. In some embodiments, the cell is obtained from humans. In various embodiments, the cell that is engineered is a blood cell. Preferably, the cell is a leukocyte (e.g., a T cell), lymphocyte or any other suitable blood cell type. In some embodiments, the cell is a peripheral blood cell. In some embodiments, the cell is a tumor-infiltrating lymphocyte (TIL). In some embodiments, the cell is a T cell, B cell or NK cell. In some embodiments, the cells are human peripheral blood mononuclear cells (PBMCs). In some embodiments, the human PBMCs are CD3+ cells. In some embodiments, the human PBMCs are CD8+ cells.

In some embodiments, the cell is a T cell. In some embodiments, the T cells can express a cell surface receptor that recognizes a specific antigenic moiety on the surface of a target cell. The cell surface receptor can be a wild type or recombinant T cell receptor (TCR), a chimeric antigen receptor (CAR), or any other surface receptor capable of recognizing an antigenic moiety that is associated with the target cell. T cells can be obtained by various methods known in the art, e.g., in vitro culture of T cells (e.g., tumor infiltrating lymphocytes) isolated from patients. Genetically modified T cells can be obtained by transducing T cells (e.g., isolated from the peripheral blood of patients), with a viral vector. In some embodiments, the T cells are CD4+ T cells, CD8+ T cells, or regulatory T cells. In some embodiments, the T cells are T helper type 1 T cells and T helper type 2 T cells. In some embodiments, the T cell expressing this receptor is an αβ-T cell. In alternate embodiments, the T cell expressing this receptor is a γδ-T cell. In some embodiments, the T cells are central memory T cells. In some embodiments, the T cells are effector memory T cells. In some embodiments, the T cells are naïve T cells.

In some embodiments, the cell is an NK cell. In some embodiments, preparation of the engineered cells includes one or more culture and/or preparation steps. The cells for introduction of the binding molecule, e.g., CAR, can be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

In some embodiments, the cells are stem cells, such as multipotent and pluripotent stem cells, including induced pluripotent stem cells (iPSCs). The cells can be primary cells, such as those isolated directly from a subject and/or isolated from a subject and frozen. In some embodiments, the stem cells are cultured with additional differentiation factors to obtain desired cell types (e.g., T cells).

Different cell types can be obtained from appropriate isolation methods. The isolation methods include the separation of different cell types based on the expression or presence in the cell of one or more specific molecules, such as surface markers, e.g., surface proteins, intracellular markers, or nucleic acid. In some embodiments, any known method for separation based on such markers can be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

Also provided are methods, nucleic acids, compositions, and kits, for expressing the binding molecules, and for producing the genetically engineered cells expressing such binding molecules. The genetic engineering generally involves introduction of a nucleic acid encoding the therapeutic molecule, e.g. CAR, e.g. TCR-like CAR, polypeptides, fusion proteins, into the cell, such as by retroviral transduction, transfection, or transformation. In some embodiments, gene transfer is accomplished by first stimulating the cell, such as by combining it with a stimulus that induces a response such as proliferation, survival, and/or activation, e.g., as measured by expression of a cytokine or activation marker, followed by transduction of the activated cells, and expansion in culture to numbers sufficient for clinical application.

In some embodiments, recombinant nucleic acids are transferred into cells using recombinant infectious virus particles, such as, e.g., vectors derived from simian virus 40 (SV40), adenoviruses, adeno-associated virus (AAV). In some embodiments, recombinant nucleic acids are transferred into T cells using recombinant lentiviral vectors or retroviral vectors, such as gamma-retroviral vectors. In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), or spleen focus forming virus (SFFV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In some embodiments, the vector is a lentivirus vector. In some embodiments, recombinant nucleic acids are transferred into T cells via electroporation. In some embodiments, recombinant nucleic acids are transferred into T cells via transposition. Other methods of introducing and expressing genetic material in immune cells include calcium phosphate transfection, protoplast fusion, cationic liposome-mediated transfection; tungsten particle-facilitated microparticle bombardment and strontium phosphate DNA co-precipitation. Many of these methods are descried e.g., in WO2019195486, which is incorporated herein by reference in its entirety.

Also provided are populations of engineered cells, compositions containing such cells and/or enriched for such cells, such as in which cells expressing the binding molecule make up at least 15%, 20%, 25%, 30%, 35%, 40%, 50%, 60%, 70%, 80%, 90%, 91%, 92%, 93%, 94%, 95%, 96%, 97%, 98%, 99%, or more percent of the total cells in the composition or cells of a certain type such as T cells, CD8+ or CD4+ cells.

In some embodiments, the engineered cells (e.g. CAR-T cells) are co-cultured with target cells (e.g., antigen presenting cells) for at least or about 1 hour, 2 hours, 3 hours, 4 hours, 5 hours, 6 hours, 12 hours, 18 hours, 1 day, 2 days, 3 days, or longer, such that the engineered cells (e.g., CAR-T cells) can be activated. In some embodiments, the target cells are Jurkat cells.

In some embodiments, IL-12 and modified IL-12 can be expressed by the engineered cells. For example, the fusion protein comprising the modified IL-12 described herein can be expressed on cell surface of engineered cells, e.g., when the fusion protein is a membrane-tethered protein. In some instances, the fusion protein comprising modified IL-12 described herein can be expressed and secreted, e.g., when the fusion protein is a soluble protein. The expression of IL-12 in the engineered cells provides some additional benefits. For example, it can increase production of IFN-γ, which is the most potent mediator of IL-12 actions, from NK and T cells, stimulate of growth and cytotoxicity of activated NK cells, CD8+ and CD4+ T cells, shift differentiation of CD4+ Th0 cells toward the Th1 phenotype, increase antibody-dependent cellular cytotoxicity (ADCC) against tumor cells, and induce IgG and suppression of IgE production from B cells, e.g., by at least or about 1 fold, 2 folds, 3 folds, 4 folds, 5 folds, 10 folds, or 20 folds.

In some embodiments, co-culturing with the target cells can increase cytokine (e.g., IFNγ) secretion of the engineered cells by at least or about 1 fold, 2 folds, 5 folds, 10 folds, 20 folds, 30 folds, 40 folds, 50 folds, 60 folds, 70 folds, 80 folds, 90 folds, 100 folds, 200 folds, 500 folds, 1000 folds, 2000 folds, 5000 folds, 10000 folds, or more as compared to the cytokine secretion level of the engineered cell without co-culturing.

In some embodiments, the cells are human PBMCs and engineered (e.g., transduced) to express the CAR, or antigen-binding fragment thereof.

In some embodiments, when the engineered cells are co-cultured with target cells (e.g., KRAS expressing cells), the engineered cells can increase cytokine (e.g., IFNγ) expression or secretion by at least or about 10%, 20%, 30%, 40%, 50%, 60%, 70%, 80%, 90%, 1 fold, 2 folds, 3 folds, 4 folds, 5 folds, 10 folds, 20 folds, 30 folds, 40 folds, 50 folds, 60 folds, 70 folds, 80 folds, 90 folds, 100 folds, or more. In some embodiments, when the engineered cells are co-cultured with target cells (e.g., KRAS expressing cells), the activated T cell population is increased by at least or about 10%, 20%, 30%, 40%, 50%, 60%, 70%, 80%, 90%, 1 fold, 2 folds, 3 folds, 4 folds, 5 folds, 10 folds, 20 folds, 50 folds, 100 folds, or more. In some embodiments, the T cell activation status can be measured by CD69 expression levels.

### Recombinant Vectors

The present disclosure also provides recombinant vectors (e.g., an expression vectors) that include an isolated polynucleotide disclosed herein (e.g., a polynucleotide that encodes a polypeptide disclosed herein), host cells into which are introduced the recombinant vectors (i.e., such that the host cells contain the polynucleotide and/or a vector comprising the polynucleotide), and the production of recombinant antibody polypeptides or fragments thereof by recombinant techniques.

As used herein, a “vector” is any construct capable of delivering one or more polynucleotide(s) of interest to a host cell when the vector is introduced to the host cell. An “expression vector” is capable of delivering and expressing the one or more polynucleotide(s) of interest as an encoded polypeptide in a host cell into which the expression vector has been introduced. Thus, in an expression vector, the polynucleotide of interest is positioned for expression in the vector by being operably linked with regulatory elements such as a promoter, enhancer, and/or a poly-A tail, either within the vector or in the genome of the host cell at or near or flanking the integration site of the polynucleotide of interest such that the polynucleotide of interest will be translated in the host cell introduced with the expression vector.

A vector can be introduced into the host cell by methods known in the art, e.g., electroporation, chemical transfection (e.g., DEAE-dextran), transformation, transfection, and infection and/or transduction (e.g., with recombinant virus). Thus, non-limiting examples of vectors include viral vectors (which can be used to generate recombinant virus), naked DNA or RNA, plasmids, cosmids, phage vectors, and DNA or RNA expression vectors associated with cationic condensing agents.

In some implementations, a polynucleotide disclosed herein (e.g., a polynucleotide that encodes a polypeptide disclosed herein) is introduced using a viral expression system (e.g., vaccinia or other pox virus, retrovirus, or adenovirus), which may involve the use of a non-pathogenic (defective), replication competent virus, or may use a replication defective virus. In the latter case, viral propagation generally will occur only in complementing virus packaging cells. Suitable systems are disclosed, for example, in Fisher-Hoch et al., 1989, Proc. Natl. Acad. Sci. USA 86:317-321; Flexner et al., 1989, Ann. N.Y. Acad Sci. 569:86-103; Flexner et al., 1990, Vaccine, 8:17-21; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner-Biotechniques, 6:616-627, 1988; Rosenfeld et al., 1991, Science, 252:431-434; Kolls et al., 1994, Proc. Natl. Acad. Sci. USA, 91:215-219; Kass-Eisler et al., 1993, Proc. Natl. Acad. Sci. USA, 90:11498-11502; Guzman et al., 1993, Circulation, 88:2838-2848; and Guzman et al., 1993, Cir. Res., 73:1202-1207. Techniques for incorporating DNA into such expression systems are well known to those of ordinary skill in the art. The DNA may also be “naked,” as described, for example, in Ulmer et al., 1993, Science, 259:1745-1749, and Cohen, 1993, Science, 259:1691-1692. The uptake of naked DNA may be increased by coating the DNA onto biodegradable beads that are efficiently transported into the cells.

For expression, the DNA insert comprising an antibody-encoding or polypeptide-encoding polynucleotide disclosed herein can be operatively linked to an appropriate promoter (e.g., a heterologous promoter), such as the phage lambda PL promoter, the E. coli lac, trp and tac promoters, the SV40 early and late promoters and promoters of retroviral LTRs, to name a few. Other suitable promoters are known to the skilled artisan. The expression constructs can further contain sites for transcription initiation, termination and, in the transcribed region, a ribosome binding site for translation. The coding portion of the mature transcripts expressed by the constructs may include a translation initiating at the beginning and a termination codon (UAA, UGA, or UAG) appropriately positioned at the end of the polypeptide to be translated.

As indicated, the expression vectors can include at least one selectable marker. Such markers include dihydrofolate reductase or neomycin resistance for eukaryotic cell culture and tetracycline or ampicillin resistance genes for culturing in E. coli and other bacteria. Representative examples of appropriate hosts include, but are not limited to, bacterial cells, such as E. coli, Streptomyces, and Salmonella typhimurium cells; fungal cells, such as yeast cells; insect cells such as Drosophila S2 and Spodoptera Sf9 cells; animal cells such as CHO, COS, Bowes melanoma, and HK 293 cells; and plant cells. Appropriate culture mediums and conditions for the host cells described herein are known in the art.

Non-limiting vectors for use in bacteria include pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Non-limiting eukaryotic vectors include pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Non-limiting bacterial promoters suitable for use include the E. coli lacI and lacZ promoters, the T3 and T7 promoters, the gpt promoter, the lambda PR and PL promoters and the trp promoter. Suitable eukaryotic promoters include the CMV immediate early promoter, the HSV thymidine kinase promoter, the early and late SV40 promoters, the promoters of retroviral LTRs, such as those of the Rous sarcoma virus (RSV), and metallothionein promoters, such as the mouse metallothionein-I promoter.

In the yeast Saccharomyces cerevisiae, a number of vectors containing constitutive or inducible promoters such as alpha factor, alcohol oxidase, and PGH may be used. For reviews, see Ausubel et al. (1989) Current Protocols in Molecular Biology, John Wiley & Sons, New York, N.Y, and Grant et al., Methods Enzymol., 153:516-544 (1997) . . .

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods In Molecular Biology (1986), which is incorporated herein by reference in its entirety.

Transcription of DNA encoding an antibody of the present disclosure by higher eukaryotes may be increased by inserting an enhancer sequence into the vector. Enhancers are cis-acting elements of DNA, usually about from 10 to 300 bp that act to increase transcriptional activity of a promoter in a given host cell-type. Examples of enhancers include the SV40 enhancer, which is located on the late side of the replication origin at base pairs 100 to 270, the cytomegalovirus early promoter enhancer, the polyoma enhancer on the late side of the replication origin, and adenovirus enhancers.

For secretion of the translated protein into the lumen of the endoplasmic reticulum, into the periplasmic space or into the extracellular environment, appropriate secretion signals may be incorporated into the expressed polypeptide. The signals may be endogenous to the polypeptide or they may be heterologous signals.

The polypeptide (e.g., antibody) can be expressed in a modified form, such as a fusion protein (e.g., a GST-fusion) or with a histidine-tag, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties can be added to the polypeptide to facilitate purification. Such regions can be removed prior to final preparation of the polypeptide. The addition of peptide moieties to polypeptides to engender secretion or excretion, to improve stability and to facilitate purification, among others, are familiar and routine techniques in the art.

### Methods of Preparing Engineered Cells

The present disclosure provides a method or process for preparing, manufacturing and/or using the engineered cells for treatment of pathological diseases or conditions.

The cells for introduction of the protein described herein, e.g., CAR, can be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, or non-human primate. In some embodiments, the cells are isolated from mouse lymph nodes.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge. In some aspects, a washing step is accomplished by tangential flow filtration (TFF). In some embodiments, the cells are resuspended in a variety of biocompatible buffers after washing, such as, for example, Ca²⁺/Mg²⁺ free PBS. In certain embodiments, components of a blood cell sample are removed and the cells directly resuspended in culture media. In some embodiments, the methods include density-based cell separation methods, such as the preparation of white blood cells from peripheral blood by lysing the red blood cells and centrifugation through a Percoll or Ficoll gradient.

In some embodiments, the method comprises one or more steps of: e.g., isolating the T cells from a patient's blood; transducing the population T cells with a viral vector including the nucleic acid construct encoding a genetically engineered antigen receptor; expanding the transduced cells in vitro; and/or infusing the expanded cells into the patient, where the engineered T cells will seek and destroy antigen positive tumor cells. In some embodiments, the nucleic acid construct further includes a sequence encoding an inhibitory protein The method further comprises: transfection of T cells with the viral vector containing the nucleic acid construct.

In some embodiments, the methods involve introducing any vectors described herein into a cell in vitro or ex vivo. In some embodiments, the vector is a viral vector and the introducing is carried out by transduction. In some embodiments, the cell is transduced for at least or about 1 hour, 2 hours, 3 hours, 4 hours, 5 hours, 6 hours, 12 hours, 18 hours, 1 day, 2 days, 3 days, 4 days, 5 days, 6 days, 1 week, 2 weeks, 3 weeks, or longer. In some embodiments, the methods further involve introducing into the cell one or more agent, wherein each of the one or more agent is independently capable of inducing a genetic disruption of a T cell receptor alpha constant (TRAC) gene and/or a T cell receptor beta constant (TRBC) gene. In some embodiments, the one or more agent is an inhibitory nucleic acid (e.g., siRNA). In some embodiments, the one or more agent is a fusion protein comprising a DNA-targeting protein and a nuclease or an RNA-guided nuclease (e.g., a clustered regularly interspaced short palindromic nucleic acid (CRISPR)-associated nuclease).

The transfection of T cells can be achieved by using any standard method such as calcium phosphate, electroporation, liposomal mediated transfer, microinjection, biolistic particle delivery system, or any other known methods by skilled artisan. In some embodiments, transfection of T cells is performed using the calcium phosphate method.

The present disclosure provides a method to create a personalized anti-tumor immunotherapy. Genetically engineered T cells can be produced from a patient's blood cells. These engineered T cells are then reinfused into the patient as a cellular therapy product.

### Methods of Treatment

The antibodies or antigen-binding fragments thereof of the present disclosure can be used for various therapeutic purposes.

In one aspect, the disclosure provides methods for treating a cancer in a subject, methods of reducing the rate of the increase of volume of a tumor in a subject over time, methods of reducing the risk of developing a metastasis, or methods of reducing the risk of developing an additional metastasis in a subject. In some embodiments, the treatment can halt, slow, retard, or inhibit progression of a cancer. In some embodiments, the treatment can result in the reduction of in the number, severity, and/or duration of one or more symptoms of the cancer in a subject.

In one aspect, the disclosure features methods that include administering a therapeutically effective amount of engineered cells expressing CAR, to a subject in need thereof (e.g., a subject having, or identified or diagnosed as having, a cancer).

In one aspect, the disclosure features methods that include administering a therapeutically effective amount of antibodies, antigen-binding fragments thereof, or engineered cells disclosed herein to a subject in need thereof (e.g., a subject having, or identified or diagnosed as having, a cancer), e.g., breast cancer (e.g., triple-negative breast cancer), carcinoid cancer, cervical cancer, endometrial cancer, neurologic cancer, head and neck cancer, liver cancer, lung cancer, small cell lung cancer, lymphoma, melanoma, ovarian cancer, pancreatic cancer, prostate cancer, renal cancer, colorectal cancer, gastric cancer, testicular cancer, thyroid cancer, bladder cancer, urethral cancer, skin cancer or hematologic malignancy. In some embodiments, the cancer is unresectable melanoma or metastatic melanoma, non-small cell lung carcinoma (NSCLC), small cell lung cancer (SCLC), bladder cancer, gastroesophageal junction cancer or metastatic hormone-refractory prostate cancer. In some embodiments, the subject has a solid tumor. In some embodiments, the cancer is squamous cell carcinoma of the head and neck (SCCHN), renal cell carcinoma (RCC), triple-negative breast cancer (TNBC), or colorectal carcinoma. In some embodiments, the subject has Hodgkin's lymphoma. In some embodiments, the subject has triple-negative breast cancer (TNBC), gastric cancer, urothelial cancer, Merkel-cell carcinoma, or head and neck cancer. In some embodiments, the cancer is melanoma, pancreatic carcinoma, mesothelioma, glioma, hematological malignancies, especially Non-Hodgkin's lymphoma, lymphoma, chronic lymphocytic leukemia, or advanced solid tumors.

In some embodiments, the anti-KRAS/HLA antibody is designed for treating a solid tumor, pancreatic cancer, colorectal cancer, non-small cell lung cancer (NSCLC), ovarian cancer, esophageal cancer, or bile duct cancer. In some embodiments, the anti-KRAS/HLA antibody is designed for treating pancreatic adenocarcinoma, lung adenocarcinoma, colorectal adenocarcinoma, or rectal adenocarcinoma.

In some embodiments, the cancer described herein can be any cancer, including any of acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vagina, cancer of the vulva, chronic lymphocytic leukemia, chronic myeloid cancer, colon cancer, colorectal cancer, endometrial cancer, esophageal cancer, uterine cervical cancer, gastrointestinal carcinoid tumor, glioma, Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, cancer of the oropharynx, ovarian cancer, cancer of the penis, pancreatic cancer, peritoneum, omentum, and mesentery cancer, pharynx cancer, prostate cancer, rectal cancer, renal cancer, skin cancer, small intestine cancer, soft tissue cancer, stomach cancer, testicular cancer, thyroid cancer, cancer of the uterus, ureter cancer, and urinary bladder cancer. A preferred cancer is cancer is pancreatic, colorectal, lung, endometrial, ovarian, or prostate cancer. Preferably, the lung cancer is lung adenocarcinoma, the ovarian cancer is epithelial ovarian cancer, and the pancreatic cancer is pancreatic carcinoma. In another preferred embodiment, the cancer is a cancer that expresses a mutated KRAS including an amino acid sequence of SEQ ID NO: 80.

In some embodiments, the compositions and methods disclosed herein can be used for treatment of patients at risk for a cancer. Patients with cancer can be identified with various methods known in the art.

As used herein, by an “effective amount” is meant an amount or dosage sufficient to effect beneficial or desired results including halting, slowing, retarding, or inhibiting progression of a disease, e.g., an autoimmune disease or a cancer. An effective amount will vary depending upon, e.g., an age and a body weight of a subject to which the antibody, antigen binding fragment, antibody-encoding polynucleotide, vector comprising the polynucleotide, and/or compositions thereof is to be administered, a severity of symptoms and a route of administration, and thus administration can be determined on an individual basis.

An effective amount can be administered in one or more administrations. By way of example, an effective amount of an antibody or an antigen binding fragment is an amount sufficient to ameliorate, stop, stabilize, reverse, inhibit, slow and/or delay progression of an autoimmune disease or a cancer in a patient or is an amount sufficient to ameliorate, stop, stabilize, reverse, slow and/or delay proliferation of a cell (e.g., a biopsied cell, any of the cancer cells described herein, or cell line (e.g., a cancer cell line)) in vitro. As is understood in the art, an effective amount of an antibody or antigen binding fragment may vary, depending on, inter alia, patient history as well as other factors such as the type (and/or dosage) of antibody used.

Effective amounts and schedules for administering the antibodies, antibody-encoding polynucleotides, and/or compositions disclosed herein may be determined empirically, and making such determinations is within the skill in the art. Those skilled in the art will understand that the dosage that must be administered will vary depending on, for example, the mammal that will receive the antibodies, antibody-encoding polynucleotides, and/or compositions disclosed herein, the route of administration, the particular type of antibodies, antibody-encoding polynucleotides, antigen binding fragments, and/or compositions disclosed herein used and other drugs being administered to the mammal. Guidance in selecting appropriate doses for antibody or antigen binding fragment can be found in the literature on therapeutic uses of antibodies and antigen binding fragments, e.g., Handbook of Monoclonal Antibodies, Ferrone et al., eds., Noges Publications, Park Ridge, N.J., 1985, ch. 22 and pp. 303-357; Smith et al., Antibodies in Human Diagnosis and Therapy, Haber et al., eds., Raven Press, New York, 1977, pp. 365-389.

A typical daily dosage of an effective amount of an antibody or ADC is 0.01 mg/kg to 100 mg/kg. In some embodiments, the dosage can be less than 100 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, or 0.1 mg/kg. In some embodiments, the dosage can be greater than 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, 0.1 mg/kg, 0.05 mg/kg, or 0.01 mg/kg. In some embodiments, the dosage is about 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.9 mg/kg, 0.8 mg/kg, 0.7 mg/kg, 0.6 mg/kg, 0.5 mg/kg, 0.4 mg/kg, 0.3 mg/kg, 0.2 mg/kg, or 0.1 mg/kg.

In any of the methods described herein, the at least one antibody, antigen-binding fragment thereof, engineered cells, or pharmaceutical composition (e.g., any of the antibodies, antigen-binding fragments, or pharmaceutical compositions described herein) and, optionally, at least one additional therapeutic agent can be administered to the subject at least once a week (e.g., once a week, twice a week, three times a week, four times a week, once a day, twice a day, or three times a day). In some embodiments, at least two different antibodies and/or antigen-binding fragments are administered in the same composition (e.g., a liquid composition). In some embodiments, at least one antibody or antigen-binding fragment and at least one additional therapeutic agent are administered in the same composition (e.g., a liquid composition). In some embodiments, the at least one antibody or antigen-binding fragment and the at least one additional therapeutic agent are administered in two different compositions (e.g., a liquid composition containing at least one antibody or antigen-binding fragment and a solid oral composition containing at least one additional therapeutic agent). In some embodiments, the at least one additional therapeutic agent is administered as a pill, tablet, or capsule. In some embodiments, the at least one additional therapeutic agent is administered in a sustained-release oral formulation.

In some embodiments, the one or more additional therapeutic agents can be administered to the subject prior to, or after administering the at least one antibody, antigen-binding antibody fragment, or pharmaceutical composition (e.g., any of the antibodies, antigen-binding antibody fragments, or pharmaceutical compositions described herein). In some embodiments, the one or more additional therapeutic agents and the at least one antibody, antigen-binding antibody fragment, or pharmaceutical composition (e.g., any of the antibodies, antigen-binding antibody fragments, or pharmaceutical compositions described herein) are administered to the subject such that there is an overlap in the bioactive period of the one or more additional therapeutic agents and the at least one antibody or antigen-binding fragment (e.g., any of the antibodies or antigen-binding fragments described herein) in the subject.

In some embodiments, the subject can be administered the at least one antibody, antigen-binding antibody fragment, one engineered cell, or pharmaceutical composition (e.g., any of the antibodies, antigen-binding antibody fragments, engineered cells, or pharmaceutical compositions described herein) over an extended period of time (e.g., over a period of at least 1 week, 2 weeks, 3 weeks, 1 month, 2 months, 3 months, 4 months, 5 months, 6 months, 7 months, 8 months, 9 months, 10 months, 11 months, 12 months, 1 year, 2 years, 3 years, 4 years, or 5 years). A skilled medical professional may determine the length of the treatment period using any of the methods described herein for diagnosing or following the effectiveness of treatment (e.g., the observation of at least one symptom of cancer). As described herein, a skilled medical professional can also change the identity and number (e.g., increase or decrease) of antibodies or antigen-binding antibody fragments (and/or one or more additional therapeutic agents) administered to the subject and can also adjust (e.g., increase or decrease) the dosage or frequency of administration of at least one antibody or antigen-binding antibody fragment (and/or one or more additional therapeutic agents) to the subject based on an assessment of the effectiveness of the treatment (e.g., using any of the methods described herein and known in the art).

In some embodiments, one or more additional therapeutic agents can be administered to the subject. The additional therapeutic agent can comprise one or more inhibitors selected from the group consisting of an inhibitor of B-Raf, an EGFR inhibitor, an inhibitor of a MEK, an inhibitor of ERK, an inhibitor of K-Ras, an inhibitor of c-Met, an inhibitor of anaplastic lymphoma kinase (ALK), an inhibitor of a phosphatidylinositol 3-kinase (PI3K), an inhibitor of an Akt, an inhibitor of mTOR, a dual PI3K/mTOR inhibitor, an inhibitor of Bruton's tyrosine kinase (BTK), and an inhibitor of Isocitrate dehydrogenase 1 (IDH1) and/or Isocitrate dehydrogenase 2 (IDH2). In some embodiments, the additional therapeutic agent is an inhibitor of indoleamine 2,3-dioxygenase-1 (IDO1) (e.g., epacadostat).

In some embodiments, the additional therapeutic agent can comprise one or more inhibitors selected from the group consisting of an inhibitor of LSD1, an inhibitor of MDM2, an inhibitor of BCL2, an inhibitor of CHK1, an inhibitor of activated hedgehog signaling pathway, and an agent that selectively degrades the estrogen receptor.

In some embodiments, the additional therapeutic agent can comprise one or more therapeutic agents selected from the group consisting of Trabectedin, nab-paclitaxel, Trebananib, Pazopanib, Cediranib, Palbociclib, everolimus, fluoropyrimidine, IFL, regorafenib, Reolysin, Alimta, Zykadia, Sutent, temsirolimus, axitinib, everolimus, sorafenib, Votrient, IMA-901, AGS-003, cabozantinib, Vinflunine, an Hsp90 inhibitor, Ad-GM-CSF, Temazolomide, IL-2, IFNa, vinblastine, Thalomid, dacarbazine, cyclophosphamide, lenalidomide, azacytidine, bortezomid, amrubicine, carfilzomib, pralatrexate, and enzastaurin.

In some embodiments, the additional therapeutic agent can comprise one or more therapeutic agents selected from the group consisting of an adjuvant, a TLR agonist, tumor necrosis factor (TNF) alpha, IL-1, HMGB1, an IL-10 antagonist, an IL-4 antagonist, an IL-13 antagonist, an IL-17 antagonist, an HVEM antagonist, an ICOS agonist, a treatment targeting CX3CL1, a treatment targeting CXCL9, a treatment targeting CXCL10, a treatment targeting CCL5, an LFA-1 agonist, an ICAM1 agonist, and a HER2 agonist.

In some embodiments, carboplatin, nab-paclitaxel, paclitaxel, cisplatin, pemetrexed, gemcitabine, FOLFOX, or FOLFIRI are administered to the subject.

In some embodiments, the additional therapeutic agent is an anti-OX40 antibody, an anti-PD-1 antibody, an anti-PD-L1 antibody, an anti-PD-L2 antibody, an anti-LAG-3 antibody, an anti-TIGIT antibody, an anti-BTLA antibody, an anti-CTLA-4 antibody, an anti-ICOS antibody, an anti-CD27 antibody, an anti-OX40 antibody, an anti-4-1BB antibody, an anti-CD40 antibody, and/or an anti-GITR antibody.

In one aspect, the disclosure provides a combination therapy. In some embodiments, the anti-KRAS/HLA antibody or antigen-binding fragment thereof (e.g., any antibody described herein) can be administered together with an immunomodulatory drug.

### Pharmaceutical Compositions and Routes of Administration

Also provided herein are pharmaceutical compositions that contain at least one (e.g., one, two, three, or four) of the antibodies or antigen-binding fragments described herein. Two or more (e.g., two, three, or four) of any of the antibodies or antigen-binding fragments described herein can be present in a pharmaceutical composition in any combination. The pharmaceutical compositions may be formulated in any manner known in the art.

Pharmaceutical compositions are formulated to be compatible with their intended route of administration (e.g., intravenous, intraarterial, intramuscular, intradermal, subcutaneous, or intraperitoneal). The compositions can include a sterile diluent (e.g., sterile water or saline), a fixed oil, polyethylene glycol, glycerine, propylene glycol or other synthetic solvents, antibacterial or antifungal agents, such as benzyl alcohol or methyl parabens, chlorobutanol, phenol, ascorbic acid, thimerosal, and the like, antioxidants, such as ascorbic acid or sodium bisulfite, chelating agents, such as ethylenediaminetetraacetic acid, buffers, such as acetates, citrates, or phosphates, and isotonic agents, such as sugars (e.g., dextrose), polyalcohols (e.g., mannitol or sorbitol), or salts (e.g., sodium chloride), or any combination thereof. Liposomal suspensions can also be used as pharmaceutically acceptable carriers (see, e.g., U.S. Pat. No. 4,522,811). Preparations of the compositions can be formulated and enclosed in ampules, disposable syringes, or multiple dose vials. Where required (as in, for example, injectable formulations), proper fluidity can be maintained by, for example, the use of a coating, such as lecithin, or a surfactant. Absorption of the antibody or antigen-binding fragment thereof can be prolonged by including an agent that delays absorption (e.g., aluminum monostearate and gelatin). Alternatively, controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid; Alza Corporation and Nova Pharmaceutical, Inc.).

Compositions containing one or more of any of the antibodies or antigen-binding fragments described herein can be formulated for parenteral (e.g., intravenous, intraarterial, intramuscular, intradermal, subcutaneous, or intraperitoneal) administration in dosage unit form (i.e., physically discrete units containing a predetermined quantity of active compound for ease of administration and uniformity of dosage).

Pharmaceutical compositions for parenteral administration are preferably sterile and substantially isotonic and manufactured under Good Manufacturing Practice (GMP) conditions. Pharmaceutical compositions can be provided in unit dosage form (i.e., the dosage for a single administration). Pharmaceutical compositions can be formulated using one or more physiologically acceptable carriers, diluents, excipients or auxiliaries. The formulation depends on the route of administration chosen. For injection, antibodies can be formulated in aqueous solutions, preferably in physiologically-compatible buffers to reduce discomfort at the site of injection. The solution can contain formulatory agents such as suspending, stabilizing and/or dispersing agents. Alternatively antibodies can be in lyophilized form for constitution with a suitable vehicle, e.g., sterile pyrogen-free water, before use.

Compositions including the engineered cells for administration, including pharmaceutical compositions and formulations, such as unit dose form compositions including the number of cells for administration in a given dose or fraction thereof are provided. The pharmaceutical compositions and formulations can include one or more optional pharmaceutically acceptable carrier or excipient.

Toxicity and therapeutic efficacy of compositions can be determined by standard pharmaceutical procedures in cell cultures or experimental animals (e.g., monkeys). One can, for example, determine the LD50 (the dose lethal to 50% of the population) and the ED50 (the dose therapeutically effective in 50% of the population): the therapeutic index being the ratio of LD50: ED50. Agents that exhibit high therapeutic indices are preferred. Where an agent exhibits an undesirable side effect, care should be taken to minimize potential damage (i.e., reduce unwanted side effects). Toxicity and therapeutic efficacy can be determined by other standard pharmaceutical procedures.

Data obtained from cell culture assays and animal studies can be used in formulating an appropriate dosage of any given agent for use in a subject (e.g., a human). A therapeutically effective amount of the one or more (e.g., one, two, three, or four) antibodies or antigen-binding fragments thereof (e.g., any of the antibodies or antibody fragments described herein) will be an amount that treats the disease (e.g., kills cancer cells) in a subject (e.g., a human subject identified as having cancer), or a subject identified as being at risk of developing the disease (e.g., a subject who has previously developed cancer but now has been cured), decreases the severity, frequency, and/or duration of one or more symptoms of a disease in a subject (e.g., a human). The effectiveness and dosing of any of the antibodies or antigen-binding fragments described herein can be determined by a health care professional or veterinary professional using methods known in the art, as well as by the observation of one or more symptoms of disease in a subject (e.g., a human). Certain factors may influence the dosage and timing required to effectively treat a subject (e.g., the severity of the disease or disorder, previous treatments, the general health and/or age of the subject, and the presence of other diseases).

Exemplary doses include milligram or microgram amounts of any of the antibodies or antigen-binding fragments described herein per kilogram of the subject's weight (e.g., about 1 μg/kg to about 500 mg/kg; about 100 μg/kg to about 500 mg/kg; about 100 μg/kg to about 50 mg/kg; about 10 μg/kg to about 5 mg/kg; about 10 μg/kg to about 0.5 mg/kg; or about 1 μg/kg to about 50 μg/kg). While these doses cover a broad range, one of ordinary skill in the art will understand that therapeutic agents, including antibodies and antigen-binding fragments thereof, vary in their potency, and effective amounts can be determined by methods known in the art. Typically, relatively low doses are administered at first, and the attending health care professional or veterinary professional (in the case of therapeutic application) or a researcher (when still working at the development stage) can subsequently and gradually increase the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, and the half-life of the antibody or antibody fragment in vivo.

The pharmaceutical compositions can be included in a container, pack, or dispenser together with instructions for administration. The disclosure also provides methods of manufacturing the antibodies or antigen binding fragments thereof for various uses as described herein.

### EXAMPLES

The invention is further described in the following examples, which do not limit the scope of the invention described in the claims.

### Example 1. Generating Anti-KRAS/HLA Antibodies

The MHC-1 VH/VL mice (detailed descriptions of MHC-I VH/VL mice can be found, e.g., in PCT/CN2022/081924, which is incorporated herein by reference in the entirety) were immunized with human KRAS G12V(HLA-A*03:01) protein (Kactus Biosystems, Cat #: MHC-HM418). The MHC-I VH/VL mice have humanized light chain immunoglobulin locus and humanized heavy chain immunoglobulin locus, and the mice also express a humanized major histocompatibility complex (MHC) protein complex.

When a desired immune response was achieved, antigen-specific immune cells were isolated from the immunized mice to further obtain anti-KRAS/HLA antibodies or to obtain the light chain and heavy chain variable region sequences of the anti-KRAS/HLA antibodies. For example, single cell technology (for example, using Beacon® Optofluidic System, Berkeley Lights Inc.) was used to screen and isolate plasma cells that secrete antigen-specific monoclonal antibodies, followed by reverse transcription and PCR sequencing to obtain antibody variable region sequences. The obtained variable region sequences were cloned into a vector containing a sequence encoding the human IgG constant region for antibody expression. The specificity of the expressed antibody binding to the KRAS G12V/HLA-A0301 complex can then be verified by FACS (fluorescence activated cell sorting). Exemplary antibodies obtained by this method included: 1A7, 1B6, 1C10, 1E12, 1F1, 1F3, 1F9, 1G1, 1G10, 1H2 and 2B12.

In another experiment, the phage display was performed to screen and find monoclonal antibodies that are specific to the KRAS G12V/HLA-A0301 complex. Exemplary antibody obtained by this method included P03141.

The heavy and light chain variable regions of 1A7, 1B6, 1C10, 1E12, 1F1, 1F3, 1F9, 1G1, 1G10, 1H2, 2B12, and P03141 are shown in FIG. 5. FIG. 2, FIG. 3 and FIG. 4 show the heavy and light chain CDR sequences of 1A7, 1B6, 1C10, 1E12, 1F1, 1F3, 1F9, 1G1, 1G10, 1H2, 2B12, and P03141 under Kabat definition and Chothia definition, respectively.

Various IgG1, IgG2 and IgG4 antibodies were made. With respect to the name of the antibodies, when the antibody VH/VL is connected to different isotypes, the isotype is added to the name. For example, if the VH and VL of 1A7 are connected to IgG1 constant regions, the antibody is named as 1A7-IgG1 (or 1A7). Examples of other isotypes are as follows: 1A7-IgG2, 1A7-IgG4. The constant region can also include some mutations. For example, when SI mutations (EU numbering: S239D and 1332E mutations) are introduced into the Fc region of 1A7-IgG1, the resulting antibody is named as 1A7-IgG1-SI (or 1A7-SI).

### Example 2. Binding Affinities of Anti-KRAS/HLA Antibodies

The affinities of the anti-KRAS/HLA antibodies to KRAS G12V/HLA-A0301 complex were measured by surface plasmon resonance (SPR) using Biacore™ 8K biosensor (Biacore, INC, Piscataway N.J.) equipped with pre-immobilized Protein A sensor chips.

His-tagged human KRAS G12V(HLA-A*03:01) complex protein (Kactus Biosystems, Cat #: MHC-HM418) was diluted to 2 μg/mL and then injected into the Biacore™ 8K biosensor at 10 μL/min for about 50 seconds to achieve a desired protein density. Purified anti-KRAS G12V/HLA antibodies at concentrations of 400, 200, 100, 50, 25, 12.5, 6.25, or 3.125 nM were then injected at 30 μL/min for 180 seconds. Dissociation was monitored for 400 seconds. The chip was regenerated after the last injection of each titration with Glycine (pH 1.7, 30 μL/min for 30 seconds).

Kinetic association rates (kon) and dissociation rates (koff) were obtained simultaneously by fitting the data globally to a 1:1 Langmuir binding model (Karlsson, R. Roos, H. Fagerstam, L. Petersson, B., 1994. Methods Enzymology 6. 99-110) using Biacore™ 8K Evaluation Software 3.0. Affinities were deduced from the quotient of the kinetic rate constants (KD=koff/kon).

As a person of ordinary skill in the art would understand, the same method with appropriate adjustments for parameters (e.g., antibody concentration) was performed for each tested antibody. The results for the tested antibodies are showed in the table below.

TABLE 1
Affinity test results

| Antibody | kon (1/Ms) | koff (1/s) | KD (M) |
|---|---|---|---|
| 1B6-SI | 4.06E+05 | 4.54E−04 | 1.12E−09 |
| 1C10-SI | 4.58E+05 | 1.40E−03 | 3.07E−09 |
| 1E12-SI | 1.11E+05 | 2.40E−03 | 2.15E−08 |
| 1F1-SI | 3.36E+04 | 9.43E−04 | 2.81E−08 |
| 1H2-SI | 3.00E+05 | 7.14E−04 | 2.38E−09 |

The affinity (KD) was obtained using Biacore™ 8K Evaluation Software 3.0 under steady state affinity model. The results for the tested antibodies are showed in the table below.

V2 scFv is a monoclonal antibody targeting KRAS G12V/HLA-A0301 complex described in Douglass et al., Sci. Immunol. 6, eabd5515 (2021). The VH and VL sequences of V2 scFv are shown in SEQ ID NO: 97 and SEQ ID NO: 98, respectively. V2 was obtained by linking V2 scFv analog to human IgG1 constant region. Additionally, V2-SI was obtained by introducing SI mutations into the IgG1 constant region.

TABLE 2
Affinity test results

| Antibody | KD (M) |
|---|---|
| V2-SI | 3.29E−07 |
| 1A7-SI | 5.20E−08 |
| 1G1-SI | 1.49E−08 |
| 1F3-SI | 1.25E−07 |
| 1G10-SI | 6.82E−08 |
| 2B12-SI | 4.85E−07 |

The results showed that all anti-KRAS/HLA antibodies showed good binding affinity to human KRAS G12V/HLA-A0301 complex. In particular, 1A7-SI, 1B6-SI, 1C10-SI, 1E12-SI, 1F1-SI, 1G1-SI, 1G10-SI and 1H2-SI showed better affinity to human KRAS G12V/HLA-A0301 complex than the positive control V2-SI.

In another experiment, the affinity of P03141-SI to KRAS G12V/HLA-A0301 complex was measured by bio-layer interferometry (BLI) using ForteBio Octet system equipped with pre-immobilized Protein A sensor chips.

Purified anti-KRAS G12V/HLA antibody was diluted to 5 μg/mL and then captured at 1000 rpm for about 200 seconds. The His-tagged human KRAS G12V(HLA-A*03:01) complex protein at a concentration of 200 nM was then injected at 1000 rpm for 180 seconds. Dissociation was monitored for 600 seconds. The chip was regenerated after the last injection of each titration with Glycine (pH 1.7, 1000 rpm for 30 seconds).

The results showed that the kon (1/Ms), koff (1/s), KD (M) values of P03141-SI were 1.36E+05, 3.87E-03 and 2.852E-08 respectively, indicating a good affinity to the human KRAS G12V/HLA-A0301 complex.

### Example 3. Binding Activity Verification of Anti-KRAS/HLA Antibodies to COS-7-HLA-A03 Cells Pulsed with KRAS Peptides

The binding activities of anti-KRAS/HLA antibodies to COS-7-HLA-A03 cells (COS-7 cells expressing HLA-A03 (SEQ ID NO: 109)) pulsed with KRAS G12WT (7-16) peptide (SEQ ID NO: 79), KRAS G12V (7-16) peptide (SEQ ID NO: 80), KRAS G12C (7-16) peptide (SEQ ID NO: 81) or KRAS G12D (7-16) peptide (SEQ ID NO: 82) were verified by flow cytometry.

Specifically, COS-7-HLA-A03 cells were incubated with the peptides (50 μM) for 1 hours at 37°C in 5% CO₂. After washing, cells were suspended in cold PBS containing serial dilutions of anti-KRAS/HLA antibodies (100 nM, 20 nM, 4 nM, 0.8 nM, 0.16 nM, 0.032 nM, and 0.0064 nM) for 30 minutes at 4°C, followed by an incubation with Alexa Fluor® 647 anti-human IgG Fcγ (Jackson Immuno Research Laboratories, Inc., Cat #: 109-606-170) for 30 minutes.

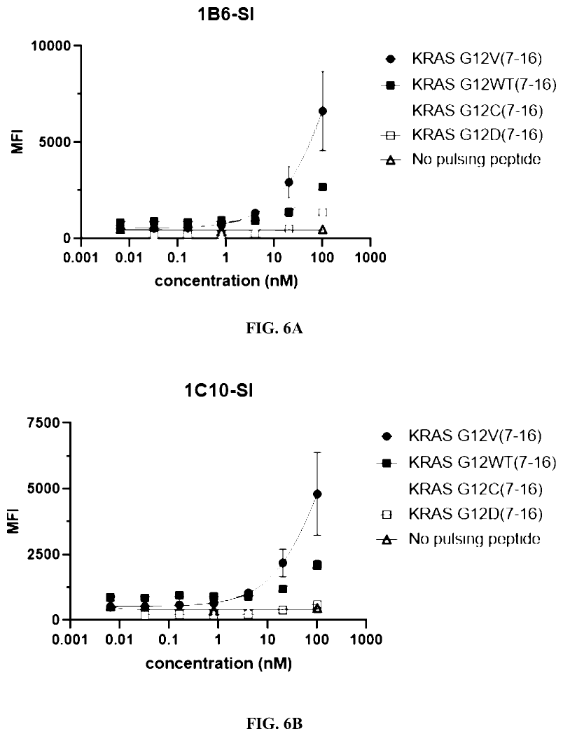

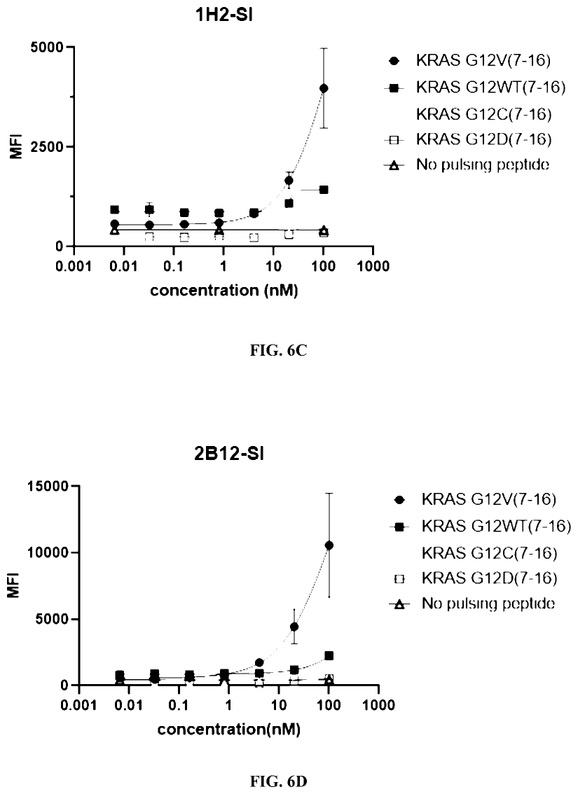

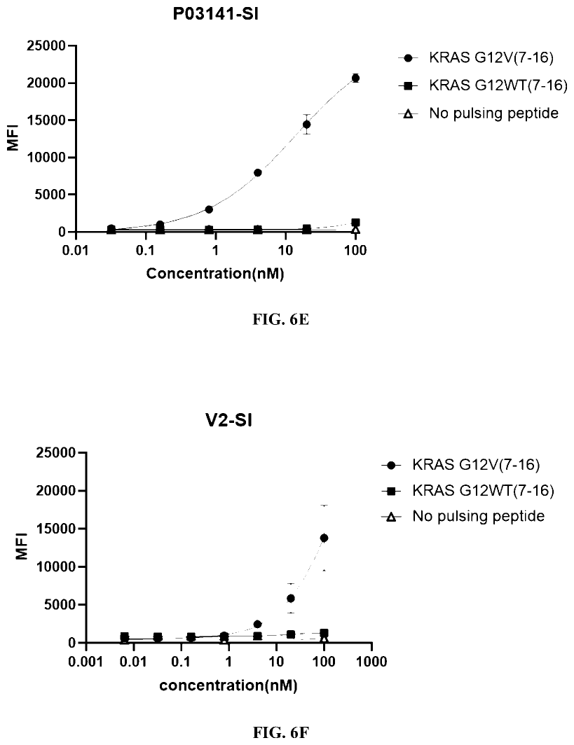

The cells were collected, and the mean fluorescence intensity (MFI) was determined. A fitting curve was obtained using antibody concentration (nM) as the X-axis and MFI as the Y-axis. The results are shown in FIGS. 6A-6D.

The results showed that anti-KRAS/HLA antibodies 1B6-SI, 1C10-SI, 1H2-SI and 2B12-SI showed specific binding to COS-7-HLA-A03 cells pulsed with KRAS G12V (7-16) peptide.

In another similar experiment, the binding activities of P03141-SI or V2-SI to COS-7-HLA-A03 cells pulsed with KRAS G12WT (7-16) peptide or KRAS G12V (7-16) peptide were verified. The results are shown in FIGS. 6E-6F, with EC50 for binding of P03141-S1 and V2-SI to KRAS G12V (7-16) peptide being 13.96 nM and 169.4 nM respectively. This indicated that P03141-SI showed specific binding to COS-7-HLA-A03 cells pulsed with KRAS G12V (7-16) peptide and its binding activity was stronger than that of V2-SI.

### Example 4. Determination of Binding Sites of Anti-KRAS/HLA Antibodies by Alanine Scanning

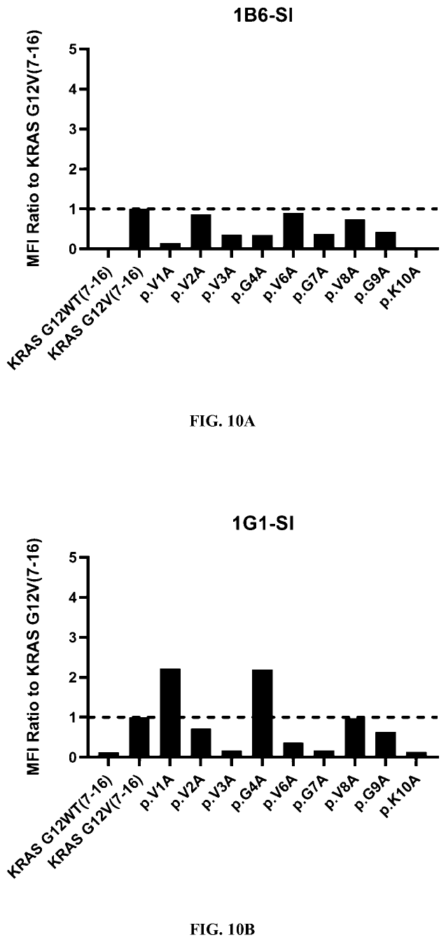

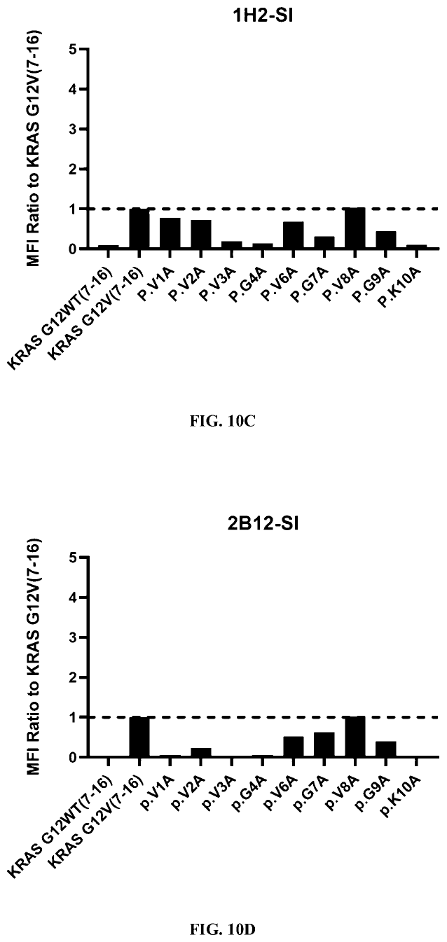

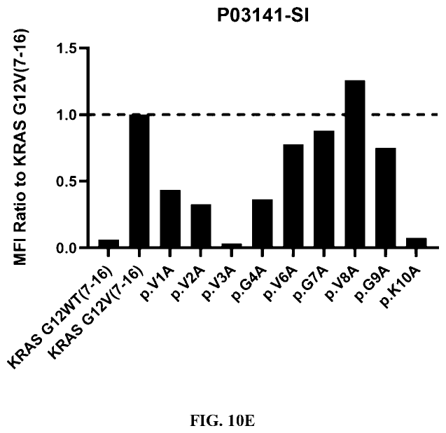

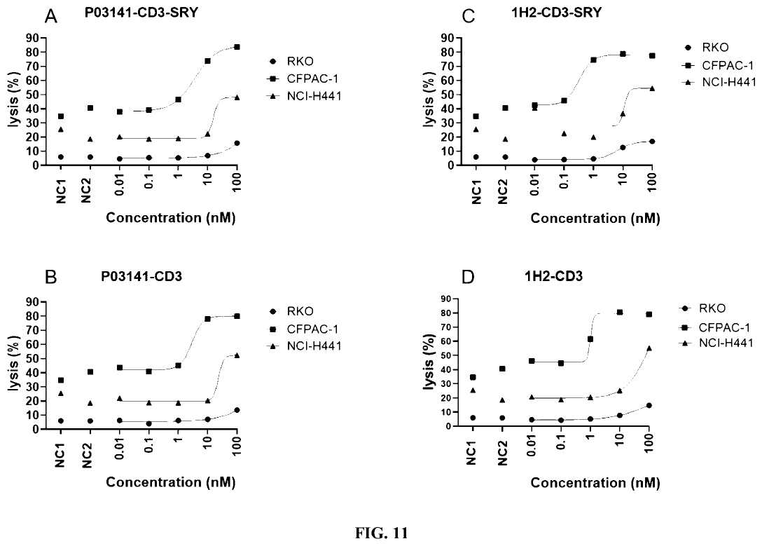

To investigate the binding sites of anti-KRAS/HLA antibodies to KRAS G12V (7-16)/HLA-A03 complex, an alanine scanning assay was used to determine the recognition epitopes of anti-KRAS/HLA antibodies. The amino acids at positions 1, 2, 3, 4, 6, 7, 8, 9 and 10 of KRAS G12V (7-16) were individually replaced with alanine to obtain a series of peptides (p.V1A, p. V2A, p. V3A, p.G4A, p. V6A, p.G7A, p. V8A, p.G9A, and p.K10A) (as shown in the table below), and flow cytometry was used to measure the binding between COS-7-HLA-A03 cells pulsed with these peptides (peptide concentration: 50 μM) and the anti-KRAS/HLA antibodies (antibody concentration: 10 μg/mL). The secondary antibody was Alexa Fluor® 647 anti-human IgG Fcγ (Jackson Immuno Research Laboratories, Inc., Cat #: 109-606-170). The mean fluorescence intensity (MFI) results are shown in FIGS. 10A-10E.

TABLE 3
Group of peptides

| Peptides | Sequence | SEQ ID NO |
|---|---|---|
| KRAS G12V(7-16) | VVVGAVGVGK | 80 |
| p.VIA | AVVGAVGVGK | 83 |
| p.V2A | VAVGAVGVGK | 84 |
| p. V3A | VVAGAVGVGK | 85 |
| p.G4A | VVVAAVGVGK | 86 |
| p.V6A | VVVGAAGVGK | 87 |
| p.G7A | VVVGAVAVGK | 88 |
| p.V8A | VVVGAVGAGK | 89 |
| p.G9A | VVVGAVGVAK | 90 |
| p.K10A | VVVGAVGVGA | 91 |

### Example 5. Cross Binding Activities of Anti-KRAS/HLA Antibodies

The binding activities of anti-KRAS/HLA antibodies to COS-7-HLA-A03 cells pulsed with KRAS G12V (7-16) peptide, and COS-7-HLA-A1101 cells (COS-7 cells expressing human HLA-A1101 (SEQ ID NO: 100)) pulsed with KRAS G12WT (7-16) peptide, KRAS G12WT (8-16) peptide (SEQ ID NO: 101), KRAS G12V (7-16) peptide, or KRAS G12V (8-16) peptide (SEQ ID NO: 102) were verified by flow cytometry.

COS-7-HLA-A03 cells or COS-7-HLA-A1101 cells were incubated with the peptides (50 μM) for 1 hours at 37°C in 5% CO₂. After washing, cells were suspended in cold PBS containing anti-KRAS/HLA antibodies (antibody concentration: 10 μg/mL) for 30 minutes at 4°C, followed by an incubation with Alexa Fluor® 647 anti-human IgG Fcγ (Jackson Immuno Research Laboratories, Inc., Cat #: 109-606-170) for 30 minutes. The cells were collected, and the MFI was determined.

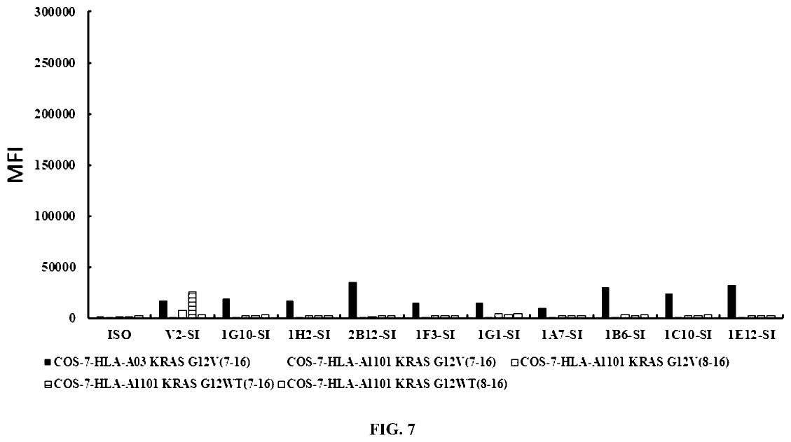

The results showed that all anti-KRAS/HLA antibodies exhibited no cross-binding with all peptides/HLA-A1101 complexes, whereas the positive control V2-SI exhibited cross-binding with the KRAS G12V (7-16)/HLA-A1101 complex. The exemplary MFI results of some of these anti-KRAS/HLA antibodies are shown in FIG. 7.

### Example 6. Binding of Anti-KRAS/HLA Antibodies to Potential Off-Target Peptides

Binding of anti-KRAS/HLA antibodies to KRAS G12V (7-16) and potential off-target peptides (sequence-similar peptides derived from MRAS protein, ERAS protein, Rab-7b protein, RhoJ protein, and mRho GTPase2 protein) was verified by flow cytometry.

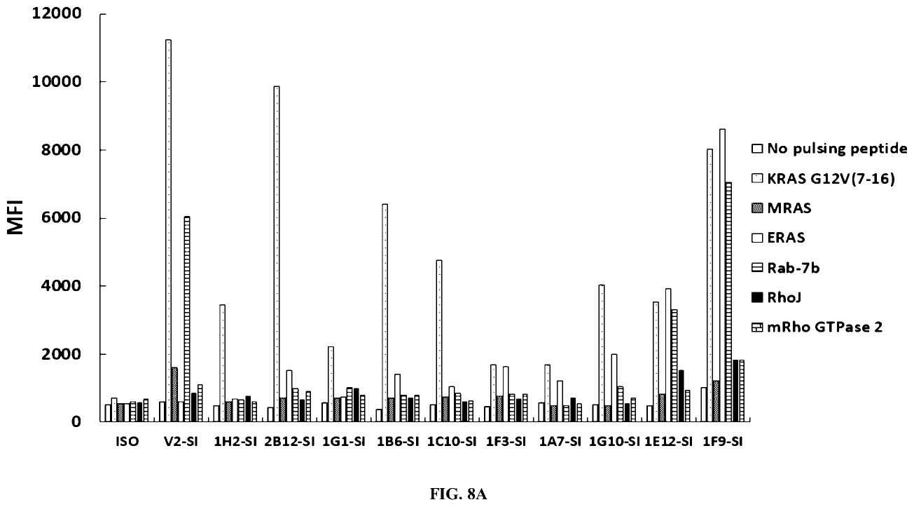

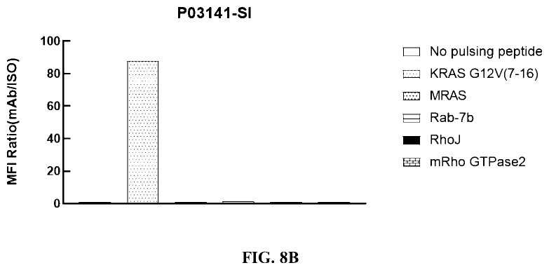

COS-7-HLA-A03 cells were incubated with 50 μM MRAS peptide, ERAS peptide, Rab-7b peptide, RhoJ peptide, and mRho GTPase2 peptide, respectively, at 37°C with 5% CO₂ for 1 hour. After washing, the cells were suspended in cold PBS and incubated with 10 μg/mL anti-KRAS G12V/HLA antibodies for 30 minutes at 4 °C. Afterwards, the cells were incubated with Alexa Fluor® 647 anti-human IgG Fcγ (Jackson Immuno Research Laboratories, Inc., Cat #: 109-606-170). The results are shown in FIG. 8A.

Sequences of the MRAS peptide, ERAS peptide, Rab-7b peptide, RhoJ peptide, and mRho GTPase2 peptide are shown in the table below.

TABLE 4
Group of peptides

| Peptides | Sequence | SEQ ID NO |
|---|---|---|
| KRAS G12V(7-16) | VVVGAVGVGK | 80 |
| MRAS | VVVGDGGVGK | 92 |
| ERAS | VVVGASGVGK | 93 |
| Rab-7b | IIVGAIGVGK | 94 |
| RhoJ | VVVGDGAVGK | 95 |
| mRho GTPase2 | KVVGARGVGK | 96 |

The results showed that anti-KRAS/HLA antibodies 1B6-SI, 1C10-SI, 1G1-SI, 1H2-SI and 2B12-SI had a good binding affinity to KRAS G12V (7-16). Further, these antibodies did not bind to MRAS peptide, ERAS peptide, Rab-7b peptide, RhoJ peptide, or mRho GTPase2 peptide. However, the positive control V2-SI also bound to Rab-7b peptide with a high MFI.

Therefore, antibody 1B6-SI, 1C10-SI, 1G1-SI, 1H2-SI and 2B12-SI showed specific binding to the KRAS G12V (7-16)/HLA-A03 complex.

In another similar experiment, the binding activity of P03141-SI to KRAS G12V (7-16) or potential off-target peptides (sequence-similar peptides derived from MRAS protein, Rab-7b protein, RhoJ protein, and mRho GTPase2 peptide) was verified. The results are shown in FIG. 8B, which indicated that P03141-SI specifically bound to KRAS G12V (7-16), but did not bind to MRAS peptide, Rab-7b peptide, RhoJ peptide, or mRho GTPase2 peptide.

### Example 7. Preparation of Anti-KRAS/CD3 Bispecific Antibodies

Different bispecific antibodies can be produced having the heavy chain and light chain sequences derived from the anti-KRAS/HLA antibodies (e.g., 1A7, 1B6, 1C10, 1E12, 1F1, 1F3, 1F9, 1G1, 1G10, 1H2, 2B12, and P03141) and the anti-CD3 antibodies described herein. Some exemplary structures of the bispecific antibody are shown in FIGS. 1A-1D.

### Structural Form I

Anti-KRAS/CD3 bispecific antibodies can be generated, which have an anti-KRAS arm comprising a heavy chain and a light chain, and an anti-CD3 arm comprising a heavy chain variable domain of an anti-CD3 heavy-chain antibody (VHH) (e.g., CD3 VHH, SEQ ID NO: 103) connected to CH2 and CH3 domains of human IgG.

Many methods can be used to reduce the chance of wrong pairing between the two heavy chains of the bispecific antibody. In the Fc region, knobs-into-holes mutations were introduced to the anti-CD3 arm heavy chain and the anti-KRAS arm heavy chain. The exemplary antibody structure is shown in FIG. 1A. Exemplary bispecific antibodies obtained include 1A7-CD3, 1B6-CD3, 1C10-CD3, 1E12-CD3, 1F1-CD3, 1G1-CD3, 1F3-CD3, 1F9-CD3, 1G10-CD3, 1H2-CD3, 2B12-CD3, P03141-CD3. For example, 1A7-CD3 is a bispecific antibody with IgG1 heavy chain constant region, the heavy chain constant region of the anti-CD3 arm (CD3) includes knob mutations, and the heavy chain constant region of the anti-KRAS arm (1A7) includes hole mutations. The sequences of the light chain constant region, the heavy chain constant region with knob mutations, and the heavy chain constant region with hole mutations are shown in SEQ ID NO: 104, SEQ ID NO: 105 and SEQ ID NO: 106, respectively.

Additionally, the VH of the anti-KRAS arm can be replaced by the VH of V2, resulting in the anti-KRAS/CD3 bispecific antibody V2-CD3, which serves as a reference antibody.

### Structural Form II

KRAS-scFv (1A7-scFv, 1B6-scFv, 1C10-scFv, 1E12-scFv, 1F1-scFv, 1G1-scFv, 1F3-scFv, 1F9-scFv, 1C10-scFv, 1H2-scFv, 2B12-scFv or P03141-scFv) and CD3-scFv (VH SEQ ID NO: 121; VL SEQ ID NO:122) can be paired to form the bispecific antibodies.

The structural form of the bispecific antibody is a BITE (Bispecific T cell Engager) molecule linked to an IgG1 Fc region (with the N297G mutation). The BITE molecule consists of two single-chain variable fragments (scFv) connected in tandem by a flexible fusion linker, wherein one scFv binds to the T cell surface protein CD3, and another scFv binds to the tumor cell surface antigen KRAS. The exemplary structure is shown in FIG. 1B. Exemplary bispecific antibodies obtained included 1A7-CD3-SLE, 1B6-CD3-SLE, 1C10-CD3-SLE, 1E12-CD3-SLE, 1F1-CD3-SLE, 1G1-CD3-SLE, 1F3-CD3-SLE, 1F9-CD3-SLE, 1G10-CD3-SLE, 1H2-CD3-SLE, 2B12-CD3-SLE, and P03141-CD3-SLE.

### Structural Form III

Anti-KRAS/CD3 bispecific antibodies can be generated, which possess a 2+1 IgG CrossFab inverted structure (exchange in CD3 binding domain, VH SEQ ID NO: 121; VL SEQ ID NO: 122) with charge modifications in KRAS binding domain, as shown in FIG. 1C. The 2+1 IgG CrossFab inverted structure comprises a first Fab molecule, a second Fab molecule, and a third Fab molecule. The first and third Fab molecules are identical and specifically bind to KRAS/HLA-A03, and the second Fab molecule binds specifically to CD3. The first Fab molecule is fused at the C-terminus of its heavy chain to the N-terminus of the heavy chain of the second Fab molecule; meanwhile, the second Fab molecule is fused at its C-terminus to the N-terminus of an Fc domain's first subunit; the third Fab molecule is fused at its C-terminus to N-terminus of this Fc domain's second subunit.

The sequences of KRAS CH1, KRAS CL, KRAS CH1-Fc, and CD3 CH1-Fc are shown in SEQ ID NO:123-126, respectively.

Exemplary bispecific antibodies obtained included 1A7-CD3-SRY, 1B6-CD3-SRY, 1C10-CD3-SRY, 1E12-CD3-SRY, 1F1-CD3-SRY, 1G1-CD3-SRY, 1F3-CD3-SRY, 1F9-CD3-SRY, 1G10-CD3-SRY, 1H2-CD3-SRY, 2B12-CD3-SRY, and P03141-CD3-SRY.

### Structural Form IV

The anti-KRAS/CD3 bispecific antibodies possess a ScDb structure, as shown in FIG. 1D, with VL and VH domains arranged in the following order: VL (KRAS)-SL-VH (CD3, e.g. SEQ ID NO: 121)-LL-VL (CD3, e.g. SEQ ID NO: 122)-SL-VH (KRAS), SL: short linker (GGGGS; SEQ ID NO:127); LL, long linker (GGGGS) 3 (SEQ ID NO:128).

Exemplary bispecific antibodies obtained included 1A7-CD3-ScDb, 1B6-CD3-ScDb, 1C10-CD3-ScDb, 1E12-CD3-ScDb, 1F1-CD3-ScDb, 1G1-CD3-ScDb, 1F3-CD3-ScDb, 1F9-CD3-ScDb, 1G10-CD3-ScDb, 1H2-CD3-ScDb, 2B12-CD3-ScDb, and P03141-CD3-ScDb.Chinese hamster ovary (CHO) cells were transfected with anti-KRAS/CD3 bispecific antibody expression vectors to express the bispecific antibodies. The CHO cell supernatant containing anti-KRAS/CD3 bispecific antibody molecules was collected and purified by Protein A affinity chromatography to obtain anti-KRAS/CD3 bispecific antibodies.

### Example 8. Cytotoxicity of Anti-KRAS/CD3 Bispecific Antibodies

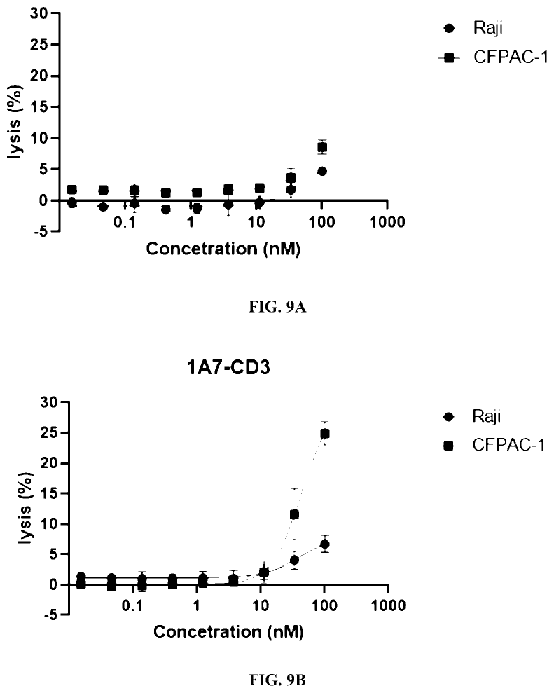

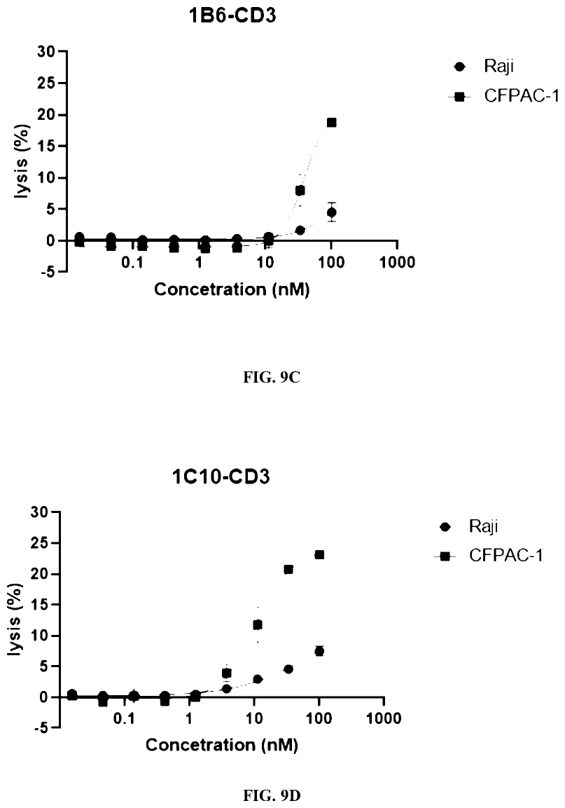

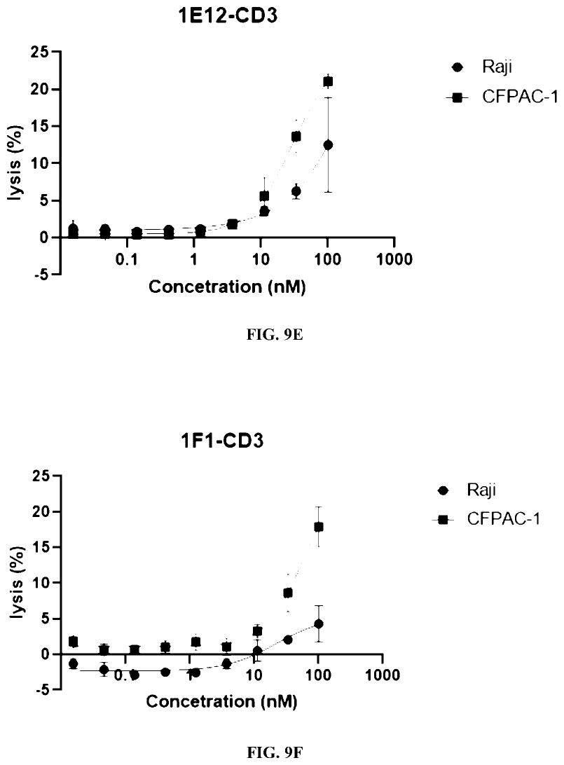

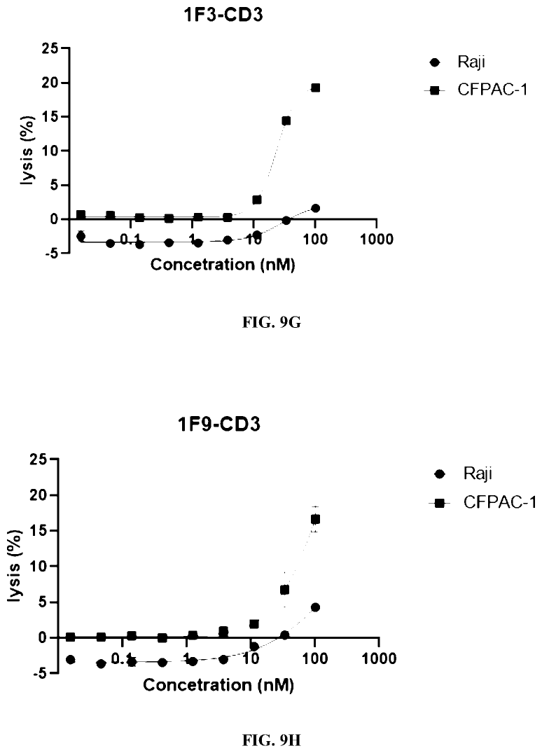

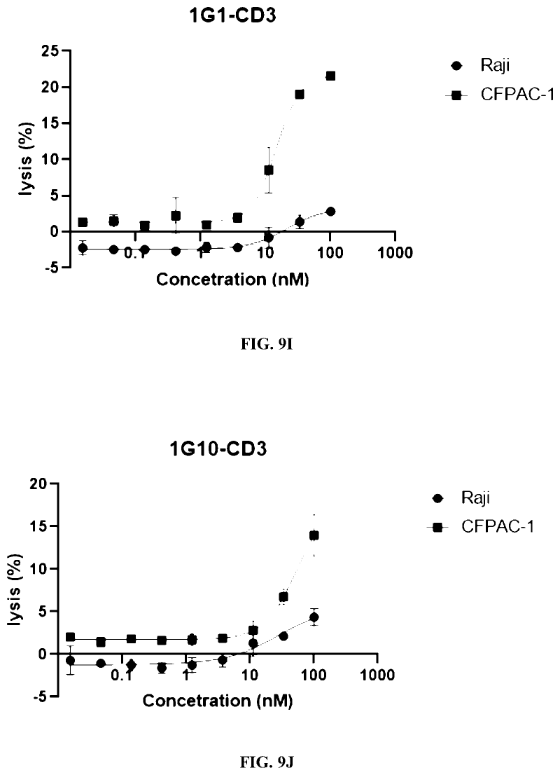

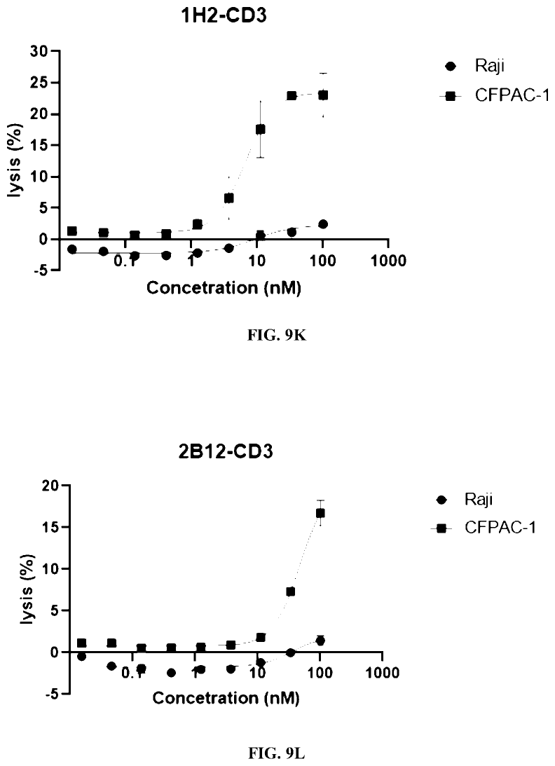

Anti-KRAS/CD3 bispecific antibodies (1A7-CD3, 1B6-CD3, 1C10-CD3, 1E12-CD3, 1F1-CD3, 1G1-CD3, 1F3-CD3, 1F9-CD3, 1G10-CD3, 1H2-CD3, 2B12-CD3 and V2-CD3) were serially diluted (3-fold) with the highest concentration at 100 nM, and then co-incubated with purified effector cells (CD3+ T cells) and target cells (Raji cells expressing wild-type KRAS (ATCC, Cat #: CCL-86)) or KRAS-driven pancreatic cancer cell lines (CFPAC-1 cells expressing endogenous KRAS G12V (ATCC, Cat #: CRL-1918)) at a 5:1 ratio (E: T) for 72 hours to test the cell killing. The lactate dehydrogenase (LDH) activity released from the cytosol of damaged cells was measured by non-radioactive colorimetric assay using Cytotoxicity Detection Kit (ROCHE, Cat #: 04744926001). The results are shown in FIGS. 9A-9L.

The results showed that V2-CD3 had a limited killing effect on CFPAC-1 (with lysis (%) less than 10%). However, anti-KRAS/CD3 bispecific antibodies 1A7-CD3, 1B6-CD3, 1C10-CD3, 1F1-CD3, 1G1-CD3, 1F3-CD3, 1F9-CD3, 1G10-CD3, 1H2-CD3, and 2B12-CD3 can kill CFPAC-1 cells, and generally did not kill Raji cells expressing wild-type KRAS.

### Example 9. In Vitro Killing Activity of Anti-KRAS/CD3 Bispecific Antibodies

### Detection of In Vitro Killing Activity by Flow Cytometry

Anti-KRAS/CD3 bispecific antibodies (P03141-CD3, P03141-CD3-SRY, 1H2-CD3, or 1H2-CD3-SRY) were serially diluted (10-fold) with the highest concentration of 100 nM, and then co-incubated with purified effector cells (CD3+ T cells) and target cells (RKO cells, CFPAC-1 cells or NCI-H441 cells) at a 10:1 ratio (E: T) for 72 hours. For negative control, no antibody or only target cells were added, and referred as NC1 and NC2 respectively. RKO cells express wild-type KRAS, while NCI-H441 cells and CFPAC-1 cells express KRAS G12V. The killing activities were verified by flow cytometry. As shown in FIG. 11, P03141-CD3, P03141-CD3-SRY, 1H2-CD3 and 1H2-CD3-SRY induced different levels of tumor cell lysis in CFPAC-1 and NCI-H441 groups, while they did not kill RKO cells even at the highest concentration.

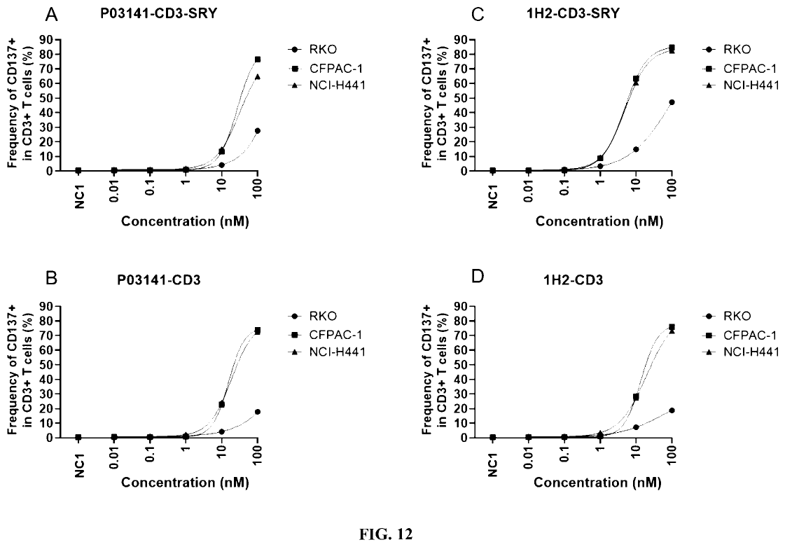

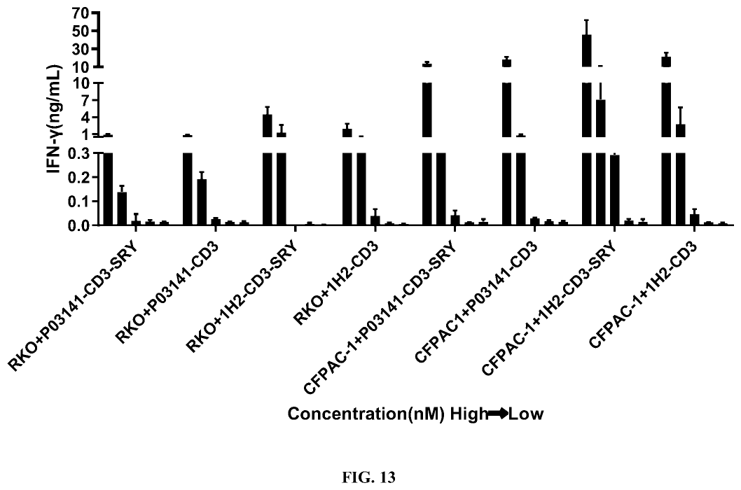

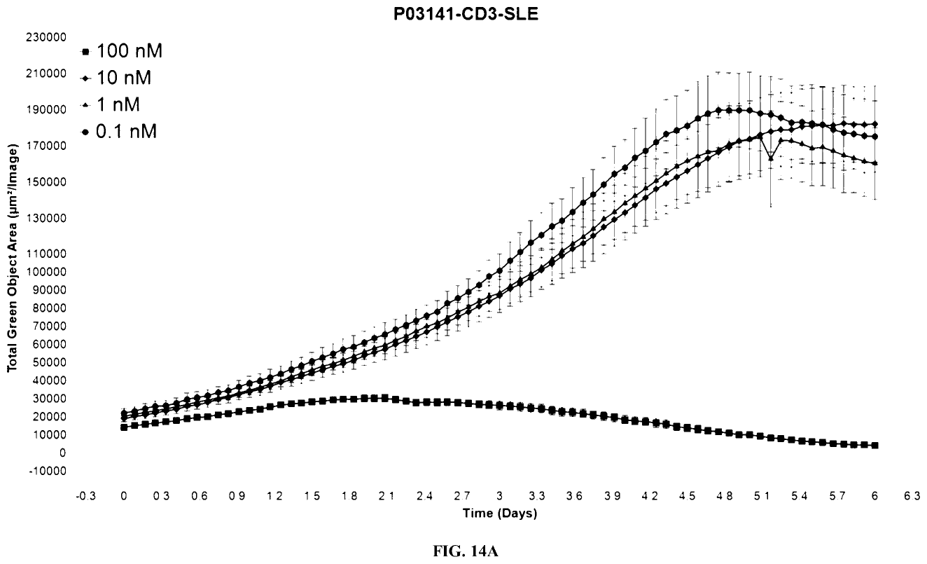

Additionally, T cell activation was determined including the proportion of CD137+ T cells by flow cytometry analysis and the secretion level of cytokine IFN-γ using BioLegend LEGEND MAX™ Human IFN-γ ELISA Kit (BioLegend, Cat #: 430107). As shown in FIG. 12 and FIG. 13, all the bispecific antibody treatment groups significantly increased CD137+ T cells, and stimulated IFN-γ secretion with a dose-dependent manner.

### Detection of In Vitro Killing Activity by Incucyte

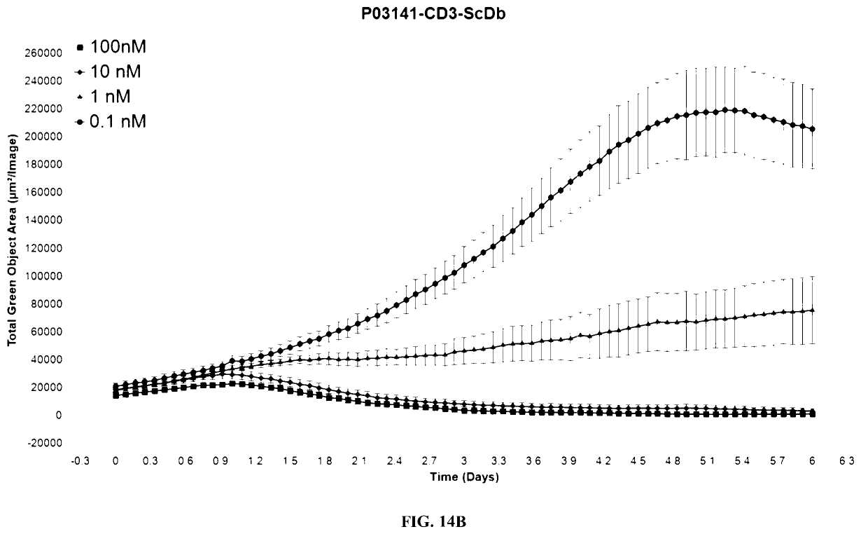

Anti-KRAS/CD3 bispecific antibodies (P03141-CD3-SLE or P03141-CD3-ScDb) were serially diluted (10-fold) with the highest concentration of 100 nM, and then co-incubated with purified effector cells (CD3+ T cells) and target cells (CFPAC-1 cells or SW620-HLA-A03 cells (engineered to express HLA-A03 and KRAS G12V)) at a 10:1 ratio (E: T) for 7 days. The killing activity was detected by IncuCyte (Sartorius AG, IncuCyte® S3). The results are shown in FIGS. 14A-14B, in which P03141-CD3-SLE and P03141-CD3-ScDb exhibited good cytotoxicities at a concentration of 1 nM, 10 nM and/or 100 nM.

### Example 10. Binding Activity of Anti-KRAS/CD3 Bispecific Antibody

The binding activities of anti-KRAS/HLA antibodies to modified Jarkat cells (Promega, Cat #: J1601) were verified by flow cytometry.

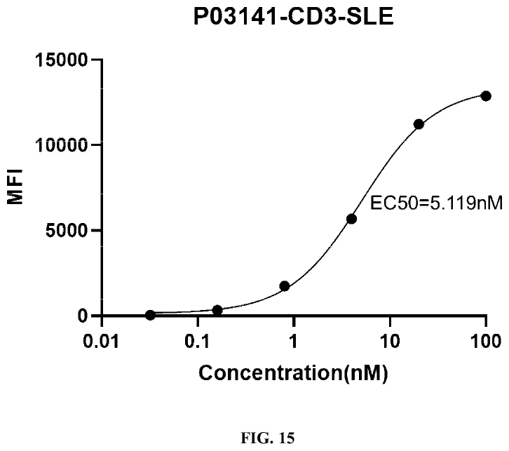

Specifically, Jurkat-Luc-OX40 cells (transfected Jurkat-Luc cells expressing human OX40 protein) were transferred to a 96-well plate. Serially diluted anti-KRAS/CD3 antibody P03141-CD3-SLE were added to the 96-well plate, and incubated at 4°C for 30 minutes. As a person of ordinary skill in the art would understand, the same method with appropriate adjustments for parameters (e.g., antibody concentration) was performed for each tested antibody. Then, the cells were incubated with the secondary antibody R-Phycoerythrin AffiniPure™ F(ab′)₂ Fragment Goat Anti-Human IgG, Fcγ fragment specific (Jackson Immuno Research Laboratories, Inc., Cat #: 109-116-098) at 4°C in the dark for 30 minutes before flow cytometry analysis. Human IgG1 protein was used as an ISO control. The cells were collected, and the mean fluorescence intensity (MFI) was determined. A fitting curve was obtained using antibody concentration (nM) as the X-axis and MFI as the Y-axis. The results are shown in FIG. 15, in which P03141-CD3-SLE exhibited good binding activity to the modified Jarkat cells.

### Example 11. Anti-Tumor Activity in NCI-H441 Xenograft Model

Anti-KRAS/CD3 bispecific antibody P03141-CD3 was tested for its effect on tumor growth in vivo in a model of lung cancer. About 5×10⁶ human PBMC were injected (by i.v. injection) in B-NDG mice (Biocytogen, Cat #: B-CM-001). On the same day, 5×10⁶ NCI-H441 cells were injected subcutaneously in the mice. When the tumor volume reached 50-150 mm³, the mice were randomly placed into different groups based on tumor volumes (6 mice per group). The mice were then injected with P03141-CD3 or an equal volume of phosphate buffer saline (PBS) by intravenous (i.v.) administration. The frequency of administration was twice a week (6 administrations in total).

The tumor volumes were measured twice a week and body weights of the mice were recorded as well. Euthanasia was performed when tumor volume of a mouse reached 2000 mm³.

The lengths of the long axis and the short axis of the tumor were measured and the volume of the tumor was calculated as 0.5×(long axis)×(short axis)². The tumor growth inhibition (TGI) was calculated using the following formula: TGI (%)=[1−(Ti−T0)/(Vi−V0)]×100%. Ti is the average tumor volume in the treatment group on day i. T0 is the average tumor volume in the treatment group on day zero. Vi is the average tumor volume in the control group on day i. V0 is the average tumor volume in the control group on day zero. T-test was performed for statistical analysis. P<0.05 is a threshold to indicate significant difference.

The results showed that the anti-KRAS/CD3 bispecific antibody P03141-CD3 exhibited good anti-tumor activity with a TGI % of 51.3% on 28 days after grouping in lung cancer model.

### Example 12. Anti-Tumor Activity

Anti-KRAS/CD3 bispecific antibodies 1B6-CD3, 1G1-CD3, 2B12-CD3, P03141-CD3 were tested for their effect on tumor growth in vivo in a model of colorectal or lung cancer. About 5×10⁶ human PBMC were injected (by i.v. injection) in B-NDG mice and then on the same day, 5×10⁶ CFPAC-1 cells or SW620 cells were injected subcutaneously in the mice. When the tumors in the mice reached a volume of 200-250 mm³, the mice were randomly placed into different groups based on tumor volumes (6 mice per group). The mice were then injected with antibodies or an equal volume of phosphate buffer saline (PBS) by intravenous (i.v.) administration. The frequency of administration was twice a week.

The results showed that all the four anti-KRAS/CD3 bispecific antibodies exhibited good anti-tumor activity in colorectal cancer or lung cancer.

### Example 13. Binding of Anti-KRAS/HLA Antibodies to Potential Off-Target Peptides

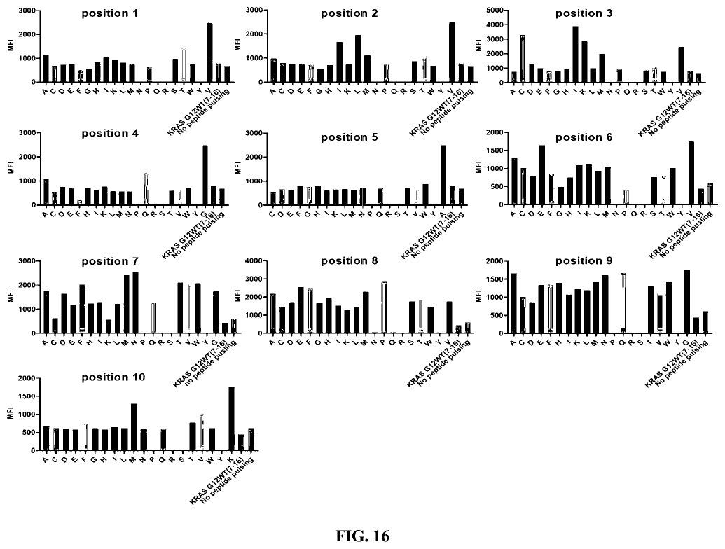

An X amino acid scanning (X-scan) assay was used to discover potential off-target peptides when anti-KRAS/HLA antibody P03141-SI recognize the KRAS G12V protein. In the X-scan experiment, the amino acids at positions 1, 2, 3, 4, 5, 6, 7, 8, 9 and 10 of KRAS G12V (7-16) were individually replaced with other different amino acids to obtain a series of peptides. Flow cytometry was then used to measure the binding between COS-7-HLA-A03 cells pulsed with these peptides (peptide concentration: 50 μM) and the anti-KRAS/HLA antibodies (antibody concentration: 10 μg/mL). The secondary antibody was Alexa Fluor® 647 anti-human IgG Fcγ. The cells were collected, and the mean fluorescence intensity (MFI) was determined. The results are shown in FIG. 16.

Then, a set of mutant peptides with binding MFI higher than the original peptide was selected. From this set, those peptides were chosen based on some factors such as their expression in humans, ability to be presented by HLA and/or high affinity with pMHC using tools like ScanProsite, MetMHCpan and/or MetCTLpan. Subsequently, the binding of anti-KRAS/HLA antibodies to these peptides (potential off-target peptide) was verified.

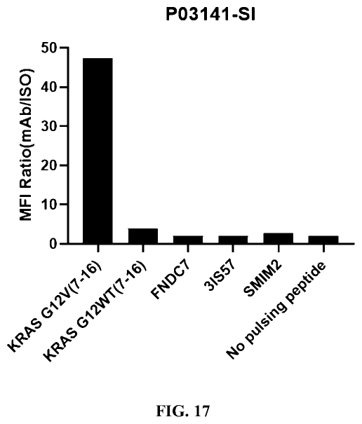

The results for the binding of P03141-SI to KRAS G12V (7-16) and three of above mentioned potential off-target peptides (referred as FNDC7 peptide, 3IS57 peptide, and SMIM2 peptide in Table 5) determined by flow cytometry are shown in FIG. 17.

TABLE 5
Group of peptides

| Peptides | Sequence | SEQ ID NO |
|---|---|---|
| KRAS G12WT(7-16) | VVVGAGGVGK | 79 |
| KRAS G12V(7-16) | VVVGAVGVGK | 80 |
| FNDC7 | ALKASFSWAR | 118 |
| 3IS57 | ALKQSATDPR | 119 |
| SMIM2 | RIDASQLPHR | 120 |

The results indicated that P03141-SI specifically bound to KRAS G12V (7-16), but did not bind to KRAS G12WT (7-16), FNDC7 peptide, 3IS57 peptide or SMIM2 peptide.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An antibody or antigen-binding fragment thereof that binds to a complex comprising a KRAS (KRAS Proto-Oncogene, GTPase) peptide and a MHC molecule, comprising:
a heavy chain variable region (VH) comprising complementarity determining regions (CDRs) 1, 2, and 3, wherein the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and
a light chain variable region (VL) comprising CDRs 1, 2, and 3, wherein the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence,
wherein the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 1, 2, 3, respectively, and the selected VL CDRs 1, 2, 3 amino acid sequences are set forth in SEQ ID NOs: 52, 53, 54, respectively;
(2) the selected VH CDRs 1, 2, 3 amino acid sequences are set forth in SEQ ID NOs: 4, 5, 6, respectively, and the selected VL CDRs 1, 2, 3 amino acid sequences are set forth in SEQ ID NOs: 55, 56, 57, respectively;
(3) the selected VH CDRs 1, 2, 3 amino acid sequences are set forth in SEQ ID NOs: 7, 8, 9, respectively, and the selected VL CDRs 1, 2, 3 amino acid sequences are set forth in SEQ ID NOs: 55, 56, 57, respectively;
(4) the selected VH CDRs 1, 2, 3 amino acid sequences are set forth in SEQ ID NOs: 10, 11, 12, respectively, and the selected VL CDRs 1, 2, 3 amino acid sequences are set forth in SEQ ID NOs: 55, 56, 57, respectively;
(5) the selected VH CDRs 1, 2, 3 amino acid sequences are set forth in SEQ ID NOs: 13, 14, 15, respectively, and the selected VL CDRs 1, 2, 3 amino acid sequences are set forth in SEQ ID NOs: 55, 56, 57, respectively;
(6) the selected VH CDRs 1, 2, 3 amino acid sequences are set forth in SEQ ID NOs: 16, 17, 18, respectively, and the selected VL CDRs 1, 2, 3 amino acid sequences are set forth in SEQ ID NOs: 55, 56, 57, respectively;
(7) the selected VH CDRs 1, 2, 3 amino acid sequences are set forth in SEQ ID NOs: 19, 20, 21, respectively, and the selected VL CDRs 1, 2, 3 amino acid sequences are set forth in SEQ ID NOs: 55, 56, 57, respectively;
(8) the selected VH CDRs 1, 2, 3 amino acid sequences are set forth in SEQ ID NOs: 22, 23, 24, respectively, and the selected VL CDRs 1, 2, 3 amino acid sequences are set forth in SEQ ID NOs: 55, 56, 57, respectively;
(9) the selected VH CDRs 1, 2, 3 amino acid sequences are set forth in SEQ ID NOs: 25, 26, 27, respectively, and the selected VL CDRs 1, 2, 3 amino acid sequences are set forth in SEQ ID NOs: 58, 59, 60, respectively;
(10) the selected VH CDRs 1, 2, 3 amino acid sequences are set forth in SEQ ID NOs: 28, 29, 30, respectively, and the selected VL CDRs 1, 2, 3 amino acid sequences are set forth in SEQ ID NOs: 52, 53, 54, respectively;
(11) the selected VH CDRs 1, 2, 3 amino acid sequences are set forth in SEQ ID NOs: 28, 29, 30, respectively, and the selected VL CDRs 1, 2, 3 amino acid sequences are set forth in SEQ ID NOs: 55, 56, 57, respectively;
(12) the selected VH CDRs 1, 2, 3 amino acid sequences are set forth in SEQ ID NOs: 31, 32, 33, respectively, and the selected VL CDRs 1, 2, 3 amino acid sequences are set forth in SEQ ID NOs: 55, 56, 57, respectively;
(13) the selected VH CDRs 1, 2, 3 amino acid sequences are set forth in SEQ ID NOs: 34, 35, 36, respectively, and the selected VL CDRs 1, 2, 3 amino acid sequences are set forth in SEQ ID NOs: 55, 56, 57, respectively;
(14) the selected VH CDRs 1, 2, 3 amino acid sequences are set forth in SEQ ID NOs: 37, 38, 39, respectively, and the selected VL CDRs 1, 2, 3 amino acid sequences are set forth in SEQ ID NOs: 55, 56, 57, respectively;
(15) the selected VH CDRs 1, 2, 3 amino acid sequences are set forth in SEQ ID NOs: 40, 41, 42, respectively, and the selected VL CDRs 1, 2, 3 amino acid sequences are set forth in SEQ ID NOs: 55, 56, 57, respectively;
(16) the selected VH CDRs 1, 2, 3 amino acid sequences are set forth in SEQ ID NOs: 43, 44, 45, respectively, and the selected VL CDRs 1, 2, 3 amino acid sequences are set forth in SEQ ID NOs: 55, 56, 57, respectively;
(17) the selected VH CDRs 1, 2, 3 amino acid sequences are set forth in SEQ ID NOs: 46, 47, 48, respectively, and the selected VL CDRs 1, 2, 3 amino acid sequences are set forth in SEQ ID NOs: 55, 56, 57, respectively;
(18) the selected VH CDRs 1, 2, 3 amino acid sequences are set forth in SEQ ID NOs: 49, 50, 51, respectively, and the selected VL CDRs 1, 2, 3 amino acid sequences are set forth in SEQ ID NOs: 58, 59, 60, respectively;
(19) the selected VH CDRs 1, 2, 3 amino acid sequences are set forth in SEQ ID NOs: 110, 111, 112, respectively, and the selected VL CDRs 1, 2, 3 amino acid sequences are set forth in SEQ ID NOs: 55, 56, 57, respectively; and
(20) the selected VH CDRs 1, 2, 3 amino acid sequences are set forth in SEQ ID NOs: 113, 114, 115, respectively, and the selected VL CDRs 1, 2, 3 amino acid sequences are set forth in SEQ ID NOs: 55, 56, 57, respectively.

2. The antibody or antigen-binding fragment thereof of claim 1, wherein the VH comprises CDRs 1, 2, 3 with the amino acid sequences set forth in SEQ ID NOs: 1, 2, and 3, respectively, and the VL comprises CDRs 1, 2, 3 with the amino acid sequences set forth in SEQ ID NOs: 52, 53, and 54, respectively according to Kabat definition.

3. The antibody or antigen-binding fragment thereof of claim 1, wherein the VH comprises CDRs 1, 2, 3 with the amino acid sequences set forth in SEQ ID NOs: 4, 5, and 6, respectively, and the VL comprises CDRs 1, 2, 3 with the amino acid sequences set forth in SEQ ID NOs: 55, 56, and 57, respectively according to Kabat definition.

4. The antibody or antigen-binding fragment thereof of claim 1, wherein the VH comprises CDRs 1, 2, 3 with the amino acid sequences set forth in SEQ ID NOs: 7, 8, and 9, respectively, and the VL comprises CDRs 1, 2, 3 with the amino acid sequences set forth in SEQ ID NOs: 55, 56, and 57, respectively according to Kabat definition.

5. The antibody or antigen-binding fragment thereof of claim 1, wherein the VH comprises CDRs 1, 2, 3 with the amino acid sequences set forth in SEQ ID NOs: 10, 11, and 12, respectively, and the VL comprises CDRs 1, 2, 3 with the amino acid sequences set forth in SEQ ID NOs: 55, 56, and 57, respectively according to Kabat definition.

6. The antibody or antigen-binding fragment thereof of claim 1, wherein the VH comprises CDRs 1, 2, 3 with the amino acid sequences set forth in SEQ ID NOs: 13, 14, and 15, respectively, and the VL comprises CDRs 1, 2, 3 with the amino acid sequences set forth in SEQ ID NOs: 55, 56, and 57, respectively according to Kabat definition.

7. The antibody or antigen-binding fragment thereof of claim 1, wherein the VH comprises CDRs 1, 2, 3 with the amino acid sequences set forth in SEQ ID NOs: 16, 17, and 18, respectively, and the VL comprises CDRs 1, 2, 3 with the amino acid sequences set forth in SEQ ID NOs: 55, 56, and 57, respectively according to Kabat definition.

8. The antibody or antigen-binding fragment thereof of claim 1, wherein the VH comprises CDRs 1, 2, 3 with the amino acid sequences set forth in SEQ ID NOs: 19, 20, and 21, respectively, and the VL comprises CDRs 1, 2, 3 with the amino acid sequences set forth in SEQ ID NOs: 55, 56, and 57, respectively according to Kabat definition.

9. The antibody or antigen-binding fragment thereof of claim 1, wherein the VH comprises CDRs 1, 2, 3 with the amino acid sequences set forth in SEQ ID NOs: 22, 23, and 24, respectively, and the VL comprises CDRs 1, 2, 3 with the amino acid sequences set forth in SEQ ID NOs: 55, 56, and 57, respectively according to Kabat definition.

10. The antibody or antigen-binding fragment thereof of claim 1, wherein the VH comprises CDRs 1, 2, 3 with the amino acid sequences set forth in SEQ ID NOs: 25, 26, and 27, respectively, and the VL comprises CDRs 1, 2, 3 with the amino acid sequences set forth in SEQ ID NOs: 58, 59, and 60, respectively according to Kabat definition.

11. The antibody or antigen-binding fragment thereof of claim 1, wherein the VH comprises CDRs 1, 2, 3 with the amino acid sequences set forth in SEQ ID NOs: 28, 29, and 30, respectively, and the VL comprises CDRs 1, 2, 3 with the amino acid sequences set forth in SEQ ID NOs: 52, 53, and 54, respectively according to Chothia definition.

12. The antibody or antigen-binding fragment thereof of claim 1, wherein the VH comprises CDRs 1, 2, 3 with the amino acid sequences set forth in SEQ ID NOs: 28, 29, and 30, respectively, and the VL comprises CDRs 1, 2, 3 with the amino acid sequences set forth in SEQ ID NOs: 55, 56, and 57, respectively according to Chothia definition.

13. The antibody or antigen-binding fragment thereof of claim 1, wherein the VH comprises CDRs 1, 2, 3 with the amino acid sequences set forth in SEQ ID NOs: 31, 32, and 33, respectively, and the VL comprises CDRs 1, 2, 3 with the amino acid sequences set forth in SEQ ID NOs: 55, 56, and 57, respectively according to Chothia definition.

14. The antibody or antigen-binding fragment thereof of claim 1, wherein the VH comprises CDRs 1, 2, 3 with the amino acid sequences set forth in SEQ ID NOs: 34, 35, and 36, respectively, and the VL comprises CDRs 1, 2, 3 with the amino acid sequences set forth in SEQ ID NOs: 55, 56, and 57, respectively according to Chothia definition.

15. The antibody or antigen-binding fragment thereof of claim 1, wherein the VH comprises CDRs 1, 2, 3 with the amino acid sequences set forth in SEQ ID NOs: 37, 38, and 39, respectively, and the VL comprises CDRs 1, 2, 3 with the amino acid sequences set forth in SEQ ID NOs: 55, 56, and 57, respectively according to Chothia definition.

16. The antibody or antigen-binding fragment thereof of claim 1, wherein the VH comprises CDRs 1, 2, 3 with the amino acid sequences set forth in SEQ ID NOs: 40, 41, and 42, respectively, and the VL comprises CDRs 1, 2, 3 with the amino acid sequences set forth in SEQ ID NOs: 55, 56, and 57, respectively according to Chothia definition.

17. The antibody or antigen-binding fragment thereof of claim 1, wherein the VH comprises CDRs 1, 2, 3 with the amino acid sequences set forth in SEQ ID NOs: 43, 44, and 45, respectively, and the VL comprises CDRs 1, 2, 3 with the amino acid sequences set forth in SEQ ID NOs: 55, 56, and 57, respectively according to Chothia definition.

18. The antibody or antigen-binding fragment thereof of claim 1, wherein the VH comprises CDRs 1, 2, 3 with the amino acid sequences set forth in SEQ ID NOs: 46, 47, and 48, respectively, and the VL comprises CDRs 1, 2, 3 with the amino acid sequences set forth in SEQ ID NOs: 55, 56, and 57, respectively according to Chothia definition.

19. The antibody or antigen-binding fragment thereof of claim 1, wherein the VH comprises CDRs 1, 2, 3 with the amino acid sequences set forth in SEQ ID NOs: 49, 50, and 51, respectively, and the VL comprises CDRs 1, 2, 3 with the amino acid sequences set forth in SEQ ID NOs: 58, 59, and 60, respectively according to Chothia definition.

20. The antibody or antigen-binding fragment thereof of claim 1, wherein the VH comprises CDRs 1, 2, 3 with the amino acid sequences set forth in SEQ ID NOs: 110, 111, and 112, respectively, and the VL comprises CDRs 1, 2, 3 with the amino acid sequences set forth in SEQ ID NOs: 55, 56, and 57, respectively according to Kabat definition.

21. The antibody or antigen-binding fragment thereof of claim 1, wherein the VH comprises CDRs 1, 2, 3 with the amino acid sequences set forth in SEQ ID NOs: 113, 114, and 115, respectively, and the VL comprises CDRs 1, 2, 3 with the amino acid sequences set forth in SEQ ID NOs: 55, 56, and 57, respectively according to Chothia definition.

22. The antibody or antigen-binding fragment thereof of any one of claims 1-21, wherein the antibody or antigen-binding fragment thereof specifically binds to a complex comprising a KRAS peptide and a MHC molecule.

23. The antibody or antigen-binding fragment thereof of any one of claims 1-22, wherein the KRAS peptide comprises a valine at a position corresponding to Gly12 of human KRAS (SEQ ID NO: 108).

24. The antibody or antigen-binding fragment thereof of any one of claim 1-23, wherein the KRAS peptide comprises or consists of a sequence that is at least 80%, 85%, 90%, 95%, or 100% identical to the amino acid sequence of SEQ ID NO: 80.

25. The antibody or antigen-binding fragment thereof of any one of claims 1-24, wherein the MHC is HLA (e.g., HLA-A3).

26. The antibody or antigen-binding fragment thereof of any one of claims 1-25, wherein the antibody or antigen-binding fragment thereof is a human or humanized antibody or antigen-binding fragment thereof, a single-chain variable fragment (scFv), a one-armed antibody and/or a multi-specific antibody (e.g., a bispecific antibody).

27. The antibody or antigen-binding fragment thereof of any one of claims 1-26, wherein the antibody or antigen-binding fragment thereof is a human IgG1 antibody or antigen-binding fragment thereof, a human IgG2 antibody or antigen-binding fragment thereof, or a human IgG4 antibody or antigen-binding fragment thereof.

28. A nucleic acid comprising a polynucleotide encoding a polypeptide comprising:
(1) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 1, 2, and 3, respectively, and wherein the VH, when paired with a light chain variable region (VL) comprising the amino acid sequence set forth in SEQ ID NO: 71 binds to a complex comprising a KRAS peptide and a MHC molecule;
(2) an immunoglobulin light chain or a fragment thereof comprising a VL comprising CDRs 1, 2, and 3 comprising the amino acid sequences set forth in SEQ ID NOs: 52, 53, and 54, respectively, and wherein the VL, when paired with a VH comprising the amino acid sequence set forth in SEQ ID NO: 61 binds to a complex comprising a KRAS peptide and a MHC molecule;
(3) an immunoglobulin heavy chain or a fragment thereof comprising a VH comprising CDRs 1, 2, and 3 comprising the amino acid sequences set forth in SEQ ID NOs: 4, 5, and 6, respectively, and wherein the VH, when paired with a VL comprising the amino acid sequence set forth in SEQ ID NO: 72 binds to a complex comprising a KRAS peptide and a MHC molecule;
(4) an immunoglobulin light chain or a fragment thereof comprising a VL comprising CDRs 1, 2, and 3 comprising the amino acid sequences set forth in SEQ ID NOs: 55, 56, and 57, respectively, and wherein the VL, when paired with a VH comprising the amino acid sequence set forth in SEQ ID NO: 62 binds to a complex comprising a KRAS peptide and a MHC molecule;
(5) an immunoglobulin heavy chain or a fragment thereof comprising a VH comprising CDRs 1, 2, and 3 comprising the amino acid sequences set forth in SEQ ID NOs: 7, 8, and 9, respectively, and wherein the VH, when paired with a VL comprising the amino acid sequence set forth in SEQ ID NO: 73 binds to a complex comprising a KRAS peptide and a MHC molecule;
(6) an immunoglobulin light chain or a fragment thereof comprising a VL comprising CDRs 1, 2, and 3 comprising the amino acid sequences set forth in SEQ ID NOs: 55, 56, and 57, respectively, and wherein the VL, when paired with a VH comprising the amino acid sequence set forth in SEQ ID NO: 63 binds to a complex comprising a KRAS peptide and a MHC molecule;
(7) an immunoglobulin heavy chain or a fragment thereof comprising a VH comprising CDRs 1, 2, and 3 comprising the amino acid sequences set forth in SEQ ID NOs: 7, 8, and 9, respectively, and wherein the VH, when paired with a VL comprising the amino acid sequence set forth in SEQ ID NO: 74 binds to a complex comprising a KRAS peptide and a MHC molecule;
(8) an immunoglobulin light chain or a fragment thereof comprising a VL comprising CDRs 1, 2, and 3 comprising the amino acid sequences set forth in SEQ ID NOs: 55, 56, and 57, respectively, and wherein the VL, when paired with a VH comprising the amino acid sequence set forth in SEQ ID NO: 64 binds to a complex comprising a KRAS peptide and a MHC molecule;
(9) an immunoglobulin heavy chain or a fragment thereof comprising a VH comprising CDRs 1, 2, and 3 comprising the amino acid sequences set forth in SEQ ID NOs: 10, 11, and 12, respectively, and wherein the VH, when paired with a VL comprising the amino acid sequence set forth in SEQ ID NO: 75 binds to a complex comprising a KRAS peptide and a MHC molecule;
(10) an immunoglobulin light chain or a fragment thereof comprising a VL comprising CDRs 1, 2, and 3 comprising the amino acid sequences set forth in SEQ ID NOs: 55, 56, and 57, respectively, and wherein the VL, when paired with a VH comprising the amino acid sequence set forth in SEQ ID NO: 65 binds to a complex comprising a KRAS peptide and a MHC molecule;
(11) an immunoglobulin heavy chain or a fragment thereof comprising a VH comprising CDRs 1, 2, and 3 comprising the amino acid sequences set forth in SEQ ID NOs: 13, 14, and 15, respectively, and wherein the VH, when paired with a VL comprising the amino acid sequence set forth in SEQ ID NO: 75 binds to a complex comprising a KRAS peptide and a MHC molecule;
(12) an immunoglobulin light chain or a fragment thereof comprising a VL comprising CDRs 1, 2, and 3 comprising the amino acid sequences set forth in SEQ ID NOs: 55, 56, and 57, respectively, and wherein the VL, when paired with a VH comprising the amino acid sequence set forth in SEQ ID NO: 66 binds to a complex comprising a KRAS peptide and a MHC molecule;
(13) an immunoglobulin heavy chain or a fragment thereof comprising a VH comprising CDRs 1, 2, and 3 comprising the amino acid sequences set forth in SEQ ID NOs: 13, 14, and 15, respectively, and wherein the VH, when paired with a VL comprising the amino acid sequence set forth in SEQ ID NO: 72 binds to a complex comprising a KRAS peptide and a MHC molecule;
(14) an immunoglobulin heavy chain or a fragment thereof comprising a VH comprising CDRs 1, 2, and 3 comprising the amino acid sequences set forth in SEQ ID NOs: 16, 17, and 18, respectively, and wherein the VH, when paired with a VL comprising the amino acid sequence set forth in SEQ ID NO: 75 binds to a complex comprising a KRAS peptide and a MHC molecule;
(15) an immunoglobulin light chain or a fragment thereof comprising a VL comprising CDRs 1, 2, and 3 comprising the amino acid sequences set forth in SEQ ID NOs: 55, 56, and 57, respectively, and wherein the VL, when paired with a VH comprising the amino acid sequence set forth in SEQ ID NO: 67 binds to a complex comprising a KRAS peptide and a MHC molecule;
(16) an immunoglobulin heavy chain or a fragment thereof comprising a VH comprising CDRs 1, 2, and 3 comprising the amino acid sequences set forth in SEQ ID NOs: 19, 20, and 21, respectively, and wherein the VH, when paired with a VL comprising the amino acid sequence set forth in SEQ ID NO: 76 binds to a complex comprising a KRAS peptide and a MHC molecule;
(17) an immunoglobulin light chain or a fragment thereof comprising a VL comprising CDRs 1, 2, and 3 comprising the amino acid sequences set forth in SEQ ID NOs: 55, 56, and 57, respectively, and wherein the VL, when paired with a VH comprising the amino acid sequence set forth in SEQ ID NO: 68 binds to a complex comprising a KRAS peptide and a MHC molecule;
(18) an immunoglobulin heavy chain or a fragment thereof comprising a VH comprising CDRs 1, 2, and 3 comprising the amino acid sequences set forth in SEQ ID NOs: 22, 23, and 24, respectively, and wherein the VH, when paired with a VL comprising the amino acid sequence set forth in SEQ ID NO: 77 binds to a complex comprising a KRAS peptide and a MHC molecule;
(19) an immunoglobulin light chain or a fragment thereof comprising a VL comprising CDRs 1, 2, and 3 comprising the amino acid sequences set forth in SEQ ID NOs: 55, 56, and 57, respectively, and wherein the VL, when paired with a VH comprising the amino acid sequence set forth in SEQ ID NO: 69 binds to a complex comprising a KRAS peptide and a MHC molecule;
(20) an immunoglobulin heavy chain or a fragment thereof comprising a VH comprising CDRs 1, 2, and 3 comprising the amino acid sequences set forth in SEQ ID NOs: 25, 26, and 27, respectively, and wherein the VH, when paired with a VL comprising the amino acid sequence set forth in SEQ ID NO: 78 binds to a complex comprising a KRAS peptide and a MHC molecule;
(21) an immunoglobulin light chain or a fragment thereof comprising a VL comprising CDRs 1, 2, and 3 comprising the amino acid sequences set forth in SEQ ID NOs: 58, 59, and 60, respectively, and wherein the VL, when paired with a VH comprising the amino acid sequence set forth in SEQ ID NO: 70 binds to a complex comprising a KRAS peptide and a MHC molecule;
(22) an immunoglobulin heavy chain or a fragment thereof comprising a heavy chain variable region (VH) comprising CDRs 1, 2, and 3 comprising the amino acid sequences set forth in SEQ ID NOs: 28, 29, and 30, respectively, and wherein the VH, when paired with a light chain variable region (VL) comprising the amino acid sequence set forth in SEQ ID NO: 71 binds to a complex comprising a KRAS peptide and a MHC molecule;
(23) an immunoglobulin heavy chain or a fragment thereof comprising a VH comprising CDRs 1, 2, and 3 comprising the amino acid sequences set forth in SEQ ID NOs: 28, 29, and 30, respectively, and wherein the VH, when paired with a VL comprising the amino acid sequence set forth in SEQ ID NO: 72 binds to a complex comprising a KRAS peptide and a MHC molecule;
(24) an immunoglobulin heavy chain or a fragment thereof comprising a VH comprising CDRs 1, 2, and 3 comprising the amino acid sequences set forth in SEQ ID NOs: 31, 32, and 33, respectively, and wherein the VH, when paired with a VL comprising the amino acid sequence set forth in SEQ ID NO: 73 binds to a complex comprising a KRAS peptide and a MHC molecule;
(25) an immunoglobulin heavy chain or a fragment thereof comprising a VH comprising CDRs 1, 2, and 3 comprising the amino acid sequences set forth in SEQ ID NOs: 31, 32, and 33, respectively, and wherein the VH, when paired with a VL comprising the amino acid sequence set forth in SEQ ID NO: 74 binds to a complex comprising a KRAS peptide and a MHC molecule;
(26) an immunoglobulin heavy chain or a fragment thereof comprising a VH comprising CDRs 1, 2, and 3 comprising the amino acid sequences set forth in SEQ ID NOs: 34, 35, and 36, respectively, and wherein the VH, when paired with a VL comprising the amino acid sequence set forth in SEQ ID NO: 75 binds to a complex comprising a KRAS peptide and a MHC molecule;
(27) an immunoglobulin heavy chain or a fragment thereof comprising a VH comprising CDRs 1, 2, and 3 comprising the amino acid sequences set forth in SEQ ID NOs: 37, 38, and 39, respectively, and wherein the VH, when paired with a VL comprising the amino acid sequence set forth in SEQ ID NO: 75 binds to a complex comprising a KRAS peptide and a MHC molecule;
(28) an immunoglobulin heavy chain or a fragment thereof comprising a VH comprising CDRs 1, 2, and 3 comprising the amino acid sequences set forth in SEQ ID NOs: 37, 38, and 39, respectively, and wherein the VH, when paired with a VL comprising the amino acid sequence set forth in SEQ ID NO: 72 binds to a complex comprising a KRAS peptide and a MHC molecule;
(29) an immunoglobulin heavy chain or a fragment thereof comprising a VH comprising CDRs 1, 2, and 3 comprising the amino acid sequences set forth in SEQ ID NOs: 40, 41, and 42, respectively, and wherein the VH, when paired with a VL comprising the amino acid sequence set forth in SEQ ID NO: 75 binds to a complex comprising a KRAS peptide and a MHC molecule;
(30) an immunoglobulin heavy chain or a fragment thereof comprising a VH comprising CDRs 1, 2, and 3 comprising the amino acid sequences set forth in SEQ ID NOs: 43, 44, and 45, respectively, and wherein the VH, when paired with a VL comprising the amino acid sequence set forth in SEQ ID NO: 76 binds to a complex comprising a KRAS peptide and a MHC molecule;
(31) an immunoglobulin heavy chain or a fragment thereof comprising a heavy chain variable region (VH) comprising CDRs 1, 2, and 3 comprising the amino acid sequences set forth in SEQ ID NOs: 46, 47, and 48, respectively, and wherein the VH, when paired with a light chain variable region (VL) comprising the amino acid sequence set forth in SEQ ID NO: 77 binds to a complex comprising a KRAS peptide and a MHC molecule;
(32) an immunoglobulin heavy chain or a fragment thereof comprising a VH comprising CDRs 1, 2, and 3 comprising the amino acid sequences set forth in SEQ ID NOs: 49, 50, and 51, respectively, and wherein the VH, when paired with a VL comprising the amino acid sequence set forth in SEQ ID NO: 78 binds to a complex comprising a KRAS peptide and a MHC molecule;
(33) an immunoglobulin heavy chain or a fragment thereof comprising a VH comprising complementarity determining regions (CDRs) 1, 2, and 3 comprising the amino acid sequences set forth in SEQ ID NOs: 110, 111, and 112, respectively, and wherein the VH, when paired with a VL comprising the amino acid sequence set forth in SEQ ID NO: 117 binds to a complex comprising a KRAS peptide and a MHC molecule;
(34) an immunoglobulin heavy chain or a fragment thereof comprising a VH comprising complementarity determining regions (CDRs) 1, 2, and 3 comprising the amino acid sequences set forth in SEQ ID NOs: 113, 114, and 115, respectively, and wherein the VH, when paired with a VL comprising the amino acid sequence set forth in SEQ ID NO: 117 binds to a complex comprising a KRAS peptide and a MHC molecule; or
(35) an immunoglobulin light chain or a fragment thereof comprising a VL comprising CDRs 1, 2, and 3 comprising the amino acid sequences set forth in SEQ ID NOs: 55, 56, and 57, respectively, and wherein the VL, when paired with a VH comprising the amino acid sequence set forth in SEQ ID NO: 116 binds to a complex comprising a KRAS peptide and a MHC molecule.

29. The nucleic acid of claim 28, wherein the VH when paired with a VL specifically binds to a complex comprising a KRAS peptide and a MHC molecule, or the VL when paired with a VH specifically binds to a complex comprising a KRAS peptide and a MHC molecule.

30. The nucleic acid of claim 28 or 29, wherein the immunoglobulin heavy chain or the fragment thereof is a human immunoglobulin heavy chain or a fragment thereof (e.g., a human IgG1 heavy chain or a fragment thereof, a human IgG2 heavy chain or a fragment thereof, or a human IgG4 heavy chain or a fragment thereof), and the immunoglobulin light chain or the fragment thereof is a human immunoglobulin light chain or a fragment thereof.

31. The nucleic acid of any one of claims 28-30, wherein the nucleic acid encodes a single-chain variable fragment (scFv), a one-armed antibody, a multi-specific antibody (e.g., a bispecific antibody), or a chimeric antigen receptor (CAR).

32. The nucleic acid of any one of claims 28-31, wherein the nucleic acid is cDNA.

33. A vector comprising one or more of the nucleic acids of any one of claims 28-32.

34. A vector comprising two of the nucleic acids of any one of claims 28-32, wherein the vector encodes the VH region and the VL region that together bind to a complex comprising a KRAS peptide and a MHC molecule.

35. A pair of vectors, wherein each vector comprises one of the nucleic acids of any one of claims 28-32, wherein together the pair of vectors encodes the VH region and the VL region that together bind to a complex comprising a KRAS peptide and a MHC molecule.

36. A cell comprising the vector of claim 33 or 34, or the pair of vectors of claim 35.

37. The cell of claim 36, wherein the cell is a CHO cell.

38. A cell comprising one or more of the nucleic acids of any one of claims 28-32.

39. A cell comprising two of the nucleic acids of any one of claims 28-32.

40. The cell of claim 39, wherein the two nucleic acids together encode the VH region and the VL region that together bind to a complex comprising a KRAS peptide and a MHC molecule.

41. A method of producing an antibody or an antigen-binding fragment thereof, the method comprising
(a) culturing the cell of any one of claims 36-40 under conditions sufficient for the cell to produce the antibody or the antigen-binding fragment; and
(b) collecting the antibody or the antigen-binding fragment produced by the cell.

42. An antibody or antigen-binding fragment thereof that binds to a complex comprising a KRAS peptide and a MHC molecule, comprising a heavy chain variable region (VH) comprising an amino acid sequence that is at least 80% identical to a selected VH sequence, and a light chain variable region (VL) comprising an amino acid sequence that is at least 80% identical to a selected VL sequence, wherein the selected VH sequence and the selected VL sequence are one of the following:
(1) the selected VH sequence is SEQ ID NO: 61, and the selected VL sequence is SEQ ID NO: 71;
(2) the selected VH sequence is SEQ ID NO: 62, and the selected VL sequence is SEQ ID NO: 72;
(3) the selected VH sequence is SEQ ID NO: 63, and the selected VL sequence is SEQ ID NO: 73;
(4) the selected VH sequence is SEQ ID NO: 64, and the selected VL sequence is SEQ ID NO: 74;
(5) the selected VH sequence is SEQ ID NO: 65, and the selected VL sequence is SEQ ID NO: 75;
(6) the selected VH sequence is SEQ ID NO: 66, and the selected VL sequence is SEQ ID NO: 75;
(7) the selected VH sequence is SEQ ID NO: 66, and the selected VL sequence is SEQ ID NO: 72;
(8) the selected VH sequence is SEQ ID NO: 67, and the selected VL sequence is SEQ ID NO: 75;
(9) the selected VH sequence is SEQ ID NO: 68, and the selected VL sequence is SEQ ID NO: 76;
(10) the selected VH sequence is SEQ ID NO: 69, and the selected VL sequence is SEQ ID NO: 77;
(11) the selected VH sequence is SEQ ID NO: 70, and the selected VL sequence is SEQ ID NO: 78; and
(12) the selected VH sequence is SEQ ID NO: 116, and the selected VL sequence is SEQ ID NO: 117.

43. The antibody or antigen-binding fragment thereof of claim 42, wherein the VH comprises the sequence of SEQ ID NO: 61 and the VL comprises the sequence of SEQ ID NO: 71.

44. The antibody or antigen-binding fragment thereof of claim 42, wherein the VH comprises the sequence of SEQ ID NO: 62 and the VL comprises the sequence of SEQ ID NO: 72.

45. The antibody or antigen-binding fragment thereof of claim 42, wherein the VH comprises the sequence of SEQ ID NO: 63 and the VL comprises the sequence of SEQ ID NO: 73.

46. The antibody or antigen-binding fragment thereof of claim 42, wherein the VH comprises the sequence of SEQ ID NO: 64 and the VL comprises the sequence of SEQ ID NO: 74.

47. The antibody or antigen-binding fragment thereof of claim 42, wherein the VH comprises the sequence of SEQ ID NO: 65 and the VL comprises the sequence of SEQ ID NO: 75.

48. The antibody or antigen-binding fragment thereof of claim 42, wherein the VH comprises the sequence of SEQ ID NO: 66 and the VL comprises the sequence of SEQ ID NO: 75.

49. The antibody or antigen-binding fragment thereof of claim 42, wherein the VH comprises the sequence of SEQ ID NO: 66 and the VL comprises the sequence of SEQ ID NO: 72.

50. The antibody or antigen-binding fragment thereof of claim 42, wherein the VH comprises the sequence of SEQ ID NO: 67 and the VL comprises the sequence of SEQ ID NO: 75.

51. The antibody or antigen-binding fragment thereof of claim 42, wherein the VH comprises the sequence of SEQ ID NO: 68 and the VL comprises the sequence of SEQ ID NO: 76.

52. The antibody or antigen-binding fragment thereof of claim 42, wherein the VH comprises the sequence of SEQ ID NO: 69 and the VL comprises the sequence of SEQ ID NO: 77.

53. The antibody or antigen-binding fragment thereof of claim 42, wherein the VH comprises the sequence of SEQ ID NO: 70 and the VL comprises the sequence of SEQ ID NO: 78.

54. The antibody or antigen-binding fragment thereof of claim 42, wherein the VH comprises the sequence of SEQ ID NO: 116 and the VL comprises the sequence of SEQ ID NO: 117.

55. An antibody or antigen-binding fragment thereof that binds to a complex comprising a KRAS peptide and a MHC molecule, comprising
a heavy chain variable region (VH) comprising VH CDR1, VH CDR2, and VH CDR3 that are identical to VH CDR1, VH CDR2, and VH CDR3 of a selected VH sequence; and
a light chain variable region (VL) comprising VL CDR1, VL CDR2, and VL CDR3 that are identical to VL CDR1, VL CDR2, and VL CDR3 of a selected VL sequence, wherein the selected VH sequence and the selected VL sequence are one of the following:
(1) the selected VH sequence is SEQ ID NO: 61, and the selected VL sequence is SEQ ID NO: 71;
(2) the selected VH sequence is SEQ ID NO: 62, and the selected VL sequence is SEQ ID NO: 72;
(3) the selected VH sequence is SEQ ID NO: 63, and the selected VL sequence is SEQ ID NO: 73;
(4) the selected VH sequence is SEQ ID NO: 64, and the selected VL sequence is SEQ ID NO: 74;
(5) the selected VH sequence is SEQ ID NO: 65, and the selected VL sequence is SEQ ID NO: 75;
(6) the selected VH sequence is SEQ ID NO: 66, and the selected VL sequence is SEQ ID NO: 75;
(7) the selected VH sequence is SEQ ID NO: 66, and the selected VL sequence is SEQ ID NO: 72;
(8) the selected VH sequence is SEQ ID NO: 67, and the selected VL sequence is SEQ ID NO: 75;
(9) the selected VH sequence is SEQ ID NO: 68, and the selected VL sequence is SEQ ID NO: 76;
(10) the selected VH sequence is SEQ ID NO: 69, and the selected VL sequence is SEQ ID NO: 77;
(11) the selected VH sequence is SEQ ID NO: 70, and the selected VL sequence is SEQ ID NO: 78; and
(12) the selected VH sequence is SEQ ID NO: 116, and the selected VL sequence is SEQ ID NO: 117.

56. The antibody or antigen-binding fragment thereof of any one of claims 42-55, wherein the antibody or antigen-binding fragment specifically binds to a complex comprising a KRAS peptide and a MHC molecule.

57. The antibody or antigen-binding fragment thereof of any one of claims 42-56, wherein the KRAS peptide comprises a valine at a position corresponding to Gly12 of human KRAS (SEQ ID NO: 108).

58. The antibody or antigen-binding fragment thereof of any one of claims 42-57, wherein the KRAS peptide comprises or consists of a sequence that is at least 80%, 85%, 90%, 95%, or 100% identical to the amino acid sequence of SEQ ID NO: 80.

59. The antibody or antigen-binding fragment thereof of any one of claims 42-58, wherein the MHC is HLA (e.g., HLA-A3).

60. The antibody or antigen-binding fragment thereof of any one of claims 42-59, wherein the antibody or antigen-binding fragment thereof is a human or humanized antibody or antigen-binding fragment thereof, a single-chain variable fragment (scFv), a one-armed antibody and/or a multi-specific antibody (e.g., a bispecific antibody).

61. The antibody or antigen-binding fragment thereof of any one of claims 42-60, wherein the antibody or antigen-binding fragment thereof is a human IgG1 antibody or antigen-binding fragment thereof, a human IgG2 antibody or antigen-binding fragment thereof, or a human IgG4 antibody or antigen-binding fragment thereof.

62. An antibody or antigen-binding fragment thereof that cross-competes with the antibody or antigen-binding fragment thereof of any one of claims 1-27 and 42-60.

63. The antibody or antigen-binding fragment thereof of any one of claims 1-27 and 42-62, wherein the antibody or antigen-binding fragment thereof comprises a fragment crystallizable region (Fc region).

64. A protein construct that binds to a complex comprising a KRAS peptide and a MHC molecule, comprising:
(1) a first functional moiety comprising an antibody or antigen-binding fragment thereof of any one of claims 1-27 and 42-62; and
(2) a second functional moiety comprising a T-cell engaging molecule.

65. The protein construct of claim 64, wherein the T-cell engaging molecule (e.g., VHH or scFv) targets human CD3.

66. The protein construct of claim 64 or 65, wherein the KRAS peptide comprises or consists of a sequence that is at least 80%, 85%, 90%, 95%, or 100% identical to the amino acid sequence of SEQ ID NO: 80.

67. The protein construct of any one of claims 64-66, wherein the MHC is HLA (e.g., HLA-A3).

68. The protein construct of any one of claims 64-67, wherein the first functional moiety and the second functional moiety are connected via a linker.

69. A protein construct, comprising:
(1) a first functional moiety comprising an antibody or antigen-binding fragment thereof of any one of claims 1-27 and 42-62;
(2) a second functional moiety comprising a T-cell engaging molecule; and
(3) a third functional moiety comprising a single chain human crystallizable fragment.

70. The protein construct of claim 69, wherein the T-cell engaging molecule is a scFv or VHH targeting human CD3.

71. The protein construct of claim 69 or 70, wherein the first functional moiety, the second functional moiety, and the third functional moiety are connected via one or more linkers.

72. The protein construct of any one of claims 69-71, wherein the KRAS peptide comprises or consists of a sequence that is at least 80%, 85%, 90%, 95%, or 100% identical to the amino acid sequence of SEQ ID NO: 80.

73. The protein construct of any one of claims 69-72, wherein the MHC is HLA (e.g., HLA-A3).

74. An antibody-drug conjugate comprising the antibody or antigen-binding fragment thereof of any one of claims 1-27 and 42-62, or the protein construct of any one of claims 64-73 covalently bound to a therapeutic agent.

75. The antibody drug conjugate of claim 74, wherein the therapeutic agent is a cytotoxic or cytostatic agent.

76. The antibody-drug conjugate of any one of claim 74 or 75, wherein the drug-to-antibody ratio (DAR) is about 4.

77. An engineered receptor comprising the antibody or antigen-binding fragment thereof of any one of claims 1-27 and 42-62.

78. The engineered receptor of claim 77, wherein the engineered receptor further comprises a transmembrane region, and an intracellular signaling domain.

79. The engineered receptor of claim 77 or 78, wherein the engineered receptor is a chimeric antigen receptor (“CAR”).

80. The engineered receptor of claim 79, wherein the engineered receptor is a chimeric T cell receptor (chimeric TCR or “cTCR”).

81. A polynucleotide encoding the engineered receptor of any one of claims 77-80.

82. A vector comprising the polynucleotide of claim 81.

83. The vector of claim 82, wherein the vector is a viral vector.

84. An engineered cell expressing the engineered receptor of any one of claims 77-80.

85. The engineered cell of claim 84, wherein the engineered cell is an immune cell.

86. The engineered cell of claim 85, wherein the immune cell is an NK cell or a T cell.

87. The engineered cell of claim 86, wherein the engineered cell is a T cell.

88. The engineered cell of claim 87, wherein the T cell is selected from the group consisting of cytotoxic T cell, a helper T cell, a natural killer T (NK-T) cell, and a γδT cell.

89. A method for producing an engineered cell, comprising introducing a vector of claim 82 or 83 into a cell in vitro or ex vivo.

90. The method of claim 89, wherein the vector is a viral vector and the introducing is carried out by transduction.

91. A method of treating a subject having cancer, the method comprising administering a therapeutically effective amount of a composition comprising the antibody or antigen-binding fragment thereof of any one of claims 1-27 and 42-62, the protein construct of any one of claims 64-73, the antibody-drug conjugate of any one of claims 74-76, or the engineered cell of any one of claims 84-88, to the subject.

92. The method of claim 91, wherein the cancer comprises one or more cancer cells that express KRAS G12V.

93. The method of claim 91 or 92, wherein the subject has a solid tumor.

94. The method of any one of claims 91-93, wherein the cancer is pancreatic cancer, colorectal cancer, non-small cell lung cancer (NSCLC), ovarian cancer, esophageal cancer, or bile duct cancer.

95. The method of any one of claims 91-94, further comprising administering a therapeutically effective amount of an anti-OX40 antibody, an anti-PD1 antibody, an anti-PDL1 antibody, an anti-PDL2 antibody, an anti-LAG-3 antibody, an anti-TIGIT antibody, an anti-CTLA-4 antibody, an anti-GITR antibody, an anti-TIM-3 antibody, an anti-4-1BB antibody, and/or an anti-CD40 antibody, to the subject.

96. A method of decreasing the rate of tumor growth, the method comprising
contacting a tumor cell with an effective amount of a composition comprising the antibody or antigen-binding fragment thereof of any one of claims 1-27 and 42-62, the protein construct of any one of claims 64-73, the antibody-drug conjugate of any one of claims 74-76, or the engineered cell of any one of claims 84-88.

97. A method of killing a tumor cell, the method comprising
contacting a tumor cell with an effective amount of a composition comprising the antibody or antigen-binding fragment thereof of any one of claims 1-27 and 42-62, the protein construct of any one of claims 64-73, the antibody-drug conjugate of any one of claims 74-76, or the engineered cell of any one of claims 84-88.

98. A method of increasing immune response in a subject, the method comprising
administering to the subject an effective amount of a composition comprising the antibody or antigen-binding fragment thereof of any one of claims 1-27 and 42-62, the protein construct of any one of claims 64-73, the antibody-drug conjugate of any one of claims 74-76, or the engineered cell of any one of claims 84-88.

99. A pharmaceutical composition comprising the antibody or antigen-binding fragment thereof of any one of claims 1-27 and 42-62, the protein construct of any one of claims 64-73, the antibody-drug conjugate of any one of claims 74-76, or the engineered cell of any one of claims 84-88, and a pharmaceutically acceptable carrier.

100. An antibody or antigen-binding fragment thereof that binds to a complex comprising a KRAS peptide and a MHC molecule, wherein the antibody or antigen-binding fragment thereof binds specifically to an epitope in the KRAS peptide, wherein the epitope is an amino acid residue corresponding to Val6 of SEQ ID NO: 80.
